(19) United States
(12) Patent Application Publication
(10) Pub. No.: US 2024/0384298 A1
(43) Pub. Date: Nov. 21, 2024

(54) NOVEL AAV CAPSIDS AND COMPOSITIONS CONTAINING SAME

(71) Applicant: The Trustees of the University of Pennsylvania, Philadelphia, PA (US)

(72) Inventors: Qiang Wang, Philadelphia, PA (US); Jenny Agnes Sidrane, Phoenixville, PA (US); James M. Wilson, Philadelphia, PA (US)

(73) Assignee: The Trustees of the University of Pennsylvania, Philadelphia, PA (US)

(21) Appl. No.: 18/696,548

(22) PCT Filed: Sep. 30, 2022
(86) PCT No.: PCT/US2022/077315

§ 371 Date: Mar. 28, 2024

Related U.S. Application Data
(60) Provisional application No. 63/251,599, filed on Oct. 2, 2021.
(60) Provisional application No. 63/343,330, filed on May 18, 2022.

(51) Int. Cl. C12N 15/86

(52) U.S. Cl. CPC C12N 15/86; C12N 2750/14122; C12N 2750/14143; C12N 2750/14152

(57) ABSTRACT

Provided herein are novel AAV capsids and recombinant AAV vectors comprising the same. In one embodiment, vectors employing a novel AAV capsid show increased transduction of a selected target tissue as compared to a prior art AAV.

### BACKGROUND OF THE INVENTION

Adeno-associated viral (AAV) vectors are safe and effective gene transfer vehicles used for several clinical indications. Recombinant AAV vectors have a vector genome lacking AAV coding sequences packaged in an AAV capsid. The AAV capsid is icosahedral in structure and is comprised of 60 of viral protein (VP) monomers (VP1, VP2, and VP3) in a 1:1:10 ratio (Xie Q, et al. Proc Natl Acad Sci USA. 2002; 99(16):10405-10). The entirety of the VP3 protein sequence (519aa) is contained within the C-terminus of both VP1 and VP2, and the shared VP3 sequences are primarily responsible for the overall capsid structure. Due to the structural flexibility of the VP1/VP2 unique regions and the low representation of VP1 and VP2 monomers relative to VP3 monomers in the assembled capsid, VP3 is the only capsid protein to be resolved via x-ray crystallography (Nam H J, et al. J Virol. 2007; 81(22):12260-71). VP3 contains nine hypervariable regions (HVRs) that are the primary source of sequence variation between AAV serotypes (Govindasamy L, et al. J Virol. 2013; 87(20):11187-99). Given their flexibility and location on the capsid surface, HVRs are largely responsible for interactions with target cells as well as with the immune system (Huang L Y, et al. J Virol. 2016; 90(11):5219-30; Raupp C, et al. J Virol. 2012; 86(17):9396-408). The structures of a number of serotypes are published (Protein Data Bank (PDB) IDs 1LP3, 4RSO, 4V86, 3UX1, 3KIC, 2QA0, 2G8G from the Research Collaboratory for Structural Bioinformatics (RCSB) database) for the structure entries for AAV2, AAVrh.8, AAV6, AAV9, AAV3B, AAV8, and AAV4, respectively).

Treatment approaches based on AAV vectors have been approved by the US Food and Drug Administration and other worldwide regulatory authorities for the treatment of Leber congenital amaurosis, lipoprotein lipase deficiency, and spinal muscular atrophy. These approved gene therapy products utilize AAV capsids isolated from natural sources as the delivery vehicle. The sequence and structural diversity of AAV capsid genes contribute to variability in viral tropism, antigenicity, and packaging efficiency that is observed between viral clades. Discovering novel capsids with an array of tissue tropisms is necessary to advance and expand the gene therapy platform. Additionally, in the last two decades, AAV engineering through modification of capsid proteins to confer increased tropism to a particular tissue

New AAV vectors for targeting to desired tissues are needed.

### SUMMARY OF THE INVENTION

In one aspect, provided herein is a recombinant adeno-associated virus comprising a capsid and having packaged therein a vector genome comprising a non-AAV exogenous nucleic acid sequence, wherein the AAV capsid is selected from: (a) an AAVhu95 capsid which is produced from a nucleic acid sequence encoding a amino acid sequence of SEQ ID NO: 2 or a predicted amino acid sequence having at least 97% identity thereto wherein amino acid positions A67, A157, T412, and S483 of SEQ ID NO: 2 are unchanged; or (b) an AAVhu96 capsid which is produced from a nucleic acid sequence encoding a amino acid sequence of SEQ ID NO: 4 or a predicted amino acid sequence having at least 97% identity thereto wherein amino acid positions A67, E157, T412, and 1483 of SEQ ID NO: 4 are unchanged. In one embodiment, the rAAV comprises an AAVhu95 capsid. In another embodiment, the rAAV comprises an AAVhu96 capsid. In some embodiments, the rAAV comprises an AAVhu95 capsid, wherein the AAVhu95 capsid is encoded by nucleic acid sequence of SEQ ID NO: 1, or a sequence at least about 91% identical thereto and encoding the amino acid sequence of SEQ ID NO: 2. In some embodiments, the rAAV comprises an AAVhu96 capsid, wherein the AAVhu96 capsid is encoded by nucleic acid sequence of SEQ ID NO: 3, or a sequence at least about 91% identical thereto and encoding the amino acid sequence of SEQ ID NO: 4. In certain embodiments, the rAAV comprises the vector genome which further comprises AAV 5′ inverted terminal repeat (ITR), an expression cassette, and an AAV 3′ ITR, wherein the expression cassette comprising a heterologous nucleic acid sequence operably linked to regulatory sequences which direct expression of a product encoded by the heterologous nucleic acid sequence in a target cell. In certain embodiments, the AAV ITR sequences are from AAV other than AAVhu95 or AAVhu96, optionally wherein the AAV ITR sequences are from AAV2.

In another aspect, provided herein is a recombinant adeno-associated virus (rAAV) comprising: (A) an AAVhu95 capsid comprising one or more of: (1) AAVhu95 capsid protein comprising: a heterogeneous population of AAVhu95 vp1 proteins selected from: vp1 proteins produced by expression from a nucleic acid sequence which encodes the predicted amino acid sequence of 1 to 736 of SEQ ID NO: 2, vp1 proteins produced from SEQ ID NO: 1, or vp1 proteins produced from a nucleic acid sequence at least 91% identical to SEQ ID NO: 1 which encodes the predicted amino acid sequence of 1 to 736 of SEQ ID NO: 2, a heterogeneous population of AAVhu95 vp2 proteins selected from: vp2 proteins produced by expression from a nucleic acid sequence which encodes the predicted amino acid sequence of at least about amino acids 138 to 736 of SEQ ID NO: 2 (or SEQ ID NO: 21), vp2 proteins produced from a sequence comprising at least nucleotides 412 to 2211 of SEQ ID NO: 1 (or SEQ ID NO: 13), or vp2 proteins produced from a nucleic acid sequence at least 91% identical to at least nucleotides 412 to 2211 of SEQ ID NO: 1 (or SEQ ID NO: 13) which encodes the predicted amino acid sequence of at least about amino acids 138 to 736 of SEQ ID NO: 2 (or SEQ ID NO: 21), a heterogeneous population of AAVhu95 vp3 proteins selected from: vp3 proteins produced by expression from a nucleic acid sequence which encodes the predicted amino acid sequence of at least about amino acids 203 to 736 of SEQ ID NO: 2 (or SEQ ID NO: 22), vp3 proteins produced from a sequence comprising at least nucleotides 607 to 2211 of SEQ ID NO: 1 (or SEQ ID NO: 14), or vp3 proteins produced from a nucleic acid sequence at least 91% identical to at least nucleotides 607 to 2211 of SEQ ID NO: 1 which encodes the predicted amino acid sequence of at least about amino acids 203 to 736 of SEQ ID NO: 2 (or SEQ ID NO: 22); and/or (2) a heterogeneous population of vp1 proteins which are the product of a nucleic acid sequence encoding the amino acid sequence of SEQ ID NO: 2, a heterogeneous population of vp2 proteins which are the product of a nucleic acid sequence encoding the amino acid sequence of at least about amino acids 138 to 736 of SEQ ID NO: 2 (or SEQ ID NO: 21), and a heterogeneous population of vp3 proteins which are the product of a nucleic acid sequence encoding at least amino acids 203 to 736 of SEQ ID NO: 2 (or SEQ ID NO: 22), wherein: the vp1, vp2 and vp3 proteins contain subpopulations with amino acid modifications comprising at least two highly deamidated asparagines (N) in asparagine—glycine pairs in SEQ ID NO: 2 and optionally further comprising subpopulations comprising other deamidated amino acids, wherein the deamidation results in an amino acid change; and (B) a vector genome in the AAVhu95 capsid, the vector genome comprising a nucleic acid molecule comprising AAV inverted terminal repeat sequences and a non-AAV nucleic acid sequence encoding a product operably linked to sequences which direct expression of the product in a target cell.

In yet another aspect, provided herein is a recombinant adeno-associated virus (rAAV) comprising: (A) an AAVhu96 capsid comprising one or more of: (1) AAVhu96 capsid protein comprising: a heterogeneous population of AAVhu96 proteins selected from: vp1 proteins produced by expression from a nucleic acid sequence which encodes the predicted amino acid sequence of 1 to 736 of SEQ ID NO: 4, vp1 proteins produced from SEQ ID NO: 3, or vp1 proteins produced from a nucleic acid sequence at least 91% identical to SEQ ID NO: 3 which encodes the predicted amino acid sequence of 1 to 736 of SEQ ID NO: 4, a heterogeneous population of AAVhu96 vp2 proteins selected from: vp2 proteins produced by expression from a nucleic acid sequence which encodes the predicted amino acid sequence of at least about amino acids 138 to 736 of SEQ ID NO: 4 (or SEQ ID NO: 23), vp2 proteins produced from a sequence comprising at least nucleotides 412 to 2211 of SEQ ID NO: 3 (or SEQ ID NO: 15), or vp2 proteins produced from a nucleic acid sequence at least 91% identical to at least nucleotides 412 to 2211 of SEQ ID NO: 3 (or SEQ ID NO: 15) which encodes the predicted amino acid sequence of at least about amino acids 138 to 736 of SEQ ID NO: 4 (or SEQ ID NO: 23), a heterogeneous population of AAVhu96 vp3 proteins selected from: vp3 proteins produced by expression from a nucleic acid sequence which encodes the predicted amino acid sequence of at least about amino acids 203 to 736 of SEQ ID NO: 4 (or SEQ ID NO: 24), vp3 proteins produced from a sequence comprising at least nucleotides 607 to 2211 of SEQ ID NO: 3 (or SEQ ID NO: 16), or vp3 proteins produced from a nucleic acid sequence at least 91% identical to at least nucleotides 607 to 2211 of SEQ ID NO: 3 (or SEQ ID NO: 16) which encodes the predicted amino acid sequence of at least about amino acids 203 to 736 of SEQ ID NO: 4 (or SEQ ID NO: 24); and/or (2) a heterogeneous population of vp1 proteins which are the product of a nucleic acid sequence encoding the amino acid sequence of SEQ ID NO: 4, a heterogeneous population of vp2 proteins which are the product of a nucleic acid sequence encoding the amino acid sequence of at least about amino acids 138 to 736 of SEQ ID NO: 4 (or SEQ ID NO: 23), and a heterogeneous population of vp3 proteins which are the product of a nucleic acid sequence encoding at least amino acids 203 to 736 of SEQ ID NO: 4 (or SEQ ID NO: 24), wherein the vp1, vp2 and vp3 proteins contain subpopulations with amino acid modifications comprising at least two highly deamidated asparagines (N) in asparagine—glycine pairs in SEQ ID NO: 4 and optionally further comprising subpopulations comprising other deamidated amino acids, wherein the deamidation results in an amino acid change; and (B) a vector genome in the AAVhu96 capsid, the vector genome comprising a nucleic acid molecule comprising AAV inverted terminal repeat sequences and a non-AAV nucleic acid sequence encoding a product operably linked to sequences which direct expression of the product in a target cell.

In another aspect, provided herein is a composition comprising at least an rAAV as described herein (e.g., rAAVhu95, rAAVhu96) and a physiologically compatible carrier, buffer, adjuvant, and/or diluent.

Provided herein are also method of transducing a cell in a central nervous system (CNS), said method comprises administering an rAAV as described herein. In certain embodiments, the method is for transducing a cardiac cell.

In one aspect, provided herein is a method of delivering of a transgene to one or more target cells of the central nervous system (CNS) of a subject comprising administering to the subject a recombinant adeno-associated virus (AAV) vector comprising an AAVhu95 capsid and a vector genome comprising the transgene operably linked to regulatory sequences that direct expression of the transgene in the target cells.

In one aspect, provided herein is a method of delivering of a transgene to one or more target cells of the central nervous system (CNS) of a subject comprising administering to the subject a recombinant adeno-associated virus (AAV) vector comprising an AAVhu96 capsid and a vector genome comprising the transgene operably linked to regulatory sequences that direct expression of the transgene in the target cells.

In another aspect provided herein is an rAAV or compositions therefor as described herein (rAAVhu95, rAAVhu96) or use thereof for delivering a gene product to a cardiac cell or to a cell of the central or peripheral nervous system.

Provided herein are also methods of generating a rAAV comprising an AAV capsid, wherein said method comprises steps of culturing production host cell comprising: (a) a molecule encoding an AAV capsid protein of AAVhu95 (amino acid sequence of SEQ ID NO: 2) or AAVhu96 (amino acid sequence SEQ ID NO: 4), (b) a functional rep gene; (c) a vector genome comprising AAV inverted terminal repeats (ITRs) and a expression cassette; and (d) sufficient helper functions to permit packaging of the vector genome into the AAV capsid protein.

In a further embodiment, a cultured production host cell containing a plasmid described herein is provided. In certain embodiments, the production cell is in a suspension cell culture.

In certain embodiments, provided herein is a recombinant nucleic acid molecule comprising a promoter and an exogenous nucleic acid sequence encoding an AAVhu95 and/or AAVhu96 capsid proteins. In some embodiments, the nucleic acid molecule comprises a nucleic acid sequence which is selected from SEQ ID NO: 1, nucleic acid sequence at least about 91% identical to SEQ ID NO: 1, SEQ ID NO: 10, or nucleic acid sequence at least about 99% identical to SEQ ID NO: 10. In other embodiments, the nucleic acid molecule comprises a nucleic acid sequence which is selected from SEQ ID NO: 3, nucleic acid sequence at least about 91% identical to SEQ ID NO: 3, SEQ ID NO: 11, or nucleic acid sequence at least about 99% identical to SEQ ID NO: 11. In certain embodiments, the nucleic acid molecule is a plasmid.

Provided herein also is a production host cell comprising a recombinant nucleic acid molecule as described herein, a nucleic acid sequence comprising an an AAV vector genome, and sufficient AAV rep functions and helper functions to permit packaging of the vector genome into the AAV capsid. IN certain embodiments, the production cell is a human cell or an insect cell, optionally wherein the production cell is HEK293 cell, HuH-7 cell, BHK cell, or Vero cell.

Other aspects and advantages of these compositions and methods are described further in the following detailed description.

### BRIEF DESCRIPTION OF THE FIGURES

FIG. 1A shows a sequence alignment of amino acids 1 to 300 of AAV capsids of clade F; AAVhu95 (SEQ ID NO: 2), AAVhu96 (SEQ ID NO: 4), AAV9 (SEQ ID NO: 6), and AAVhu68 (SEQ ID NO: 9).

FIG. 1B shows a sequence alignment of amino acids 301 to 600 of AAV capsids of clade F: AAVhu95 (SEQ ID NO: 2), AAVhu96 (SEQ ID NO: 4), AAV9 (SEQ ID NO: 6), and AAVhu68 (SEQ ID NO: 9).

FIG. 1C shows a sequence alignment of amino acids 601 to 736 of AAV capsids of clade F: AAVhu95 (SEQ ID NO: 2), AAVhu96 (SEQ ID NO: 4), AAV9 (SEQ ID NO: 6), and AAVhu68 (SEQ ID NO: 9).

FIG. 2A shows a sequence alignment of nucleotides 1 to 180 of AAV capsids of clade F: AAV9 (SEQ ID NO: 5); AAVhu68 (SEQ ID NO: 7); AAVhu68 engineered (SEQ ID NO: 8); AAVhu95 (SEQ ID NO: 10); AAVhu96 (SEQ ID NO: 11); AAVhu95 engineered (SEQ ID NO: 1); AAVhu96 (SEQ ID NO: 3).

FIG. 2B shows a sequence alignment of nucleotides 181 to 360 of AAV capsids of clade F: AAV9 (SEQ ID NO: 5); AAVhu68 (SEQ ID NO: 7); AAVhu68 engineered (SEQ ID NO: 8); AAVhu95 (SEQ ID NO: 10); AAVhu96 (SEQ ID NO: 11); AAVhu95 engineered (SEQ ID NO: 1); AAVhu96 (SEQ ID NO: 3).

FIG. 2C shows a sequence alignment of nucleotides 361 to 540 of AAV capsids of clade F: AAV9 (SEQ ID NO: 5); AAVhu68 (SEQ ID NO: 7); AAVhu68 engineered (SEQ ID NO: 8); AAVhu95 (SEQ ID NO: 10); AAVhu96 (SEQ ID NO: 11); AAVhu95 engineered (SEQ ID NO: 1); AAVhu96 (SEQ ID NO: 3).

FIG. 2D shows a sequence alignment of nucleotides 541 to 720 of AAV capsids of clade F: AAV9 (SEQ ID NO: 5); AAVhu68 (SEQ ID NO: 7); AAVhu68 engineered (SEQ ID NO: 8); AAVhu95 (SEQ ID NO: 10); AAVhu96 (SEQ ID NO: 11); AAVhu95 engineered (SEQ ID NO: 1); AAVhu96 (SEQ ID NO: 3).

FIG. 2E shows a sequence alignment of nucleotides 721 to 900 of AAV capsids of clade F: AAV9 (SEQ ID NO: 5); AAVhu68 (SEQ ID NO: 7); AAVhu68 engineered (SEQ ID NO: 8); AAVhu95 (SEQ ID NO: 10); AAVhu96 (SEQ ID NO: 11); AAVhu95 engineered (SEQ ID NO: 1); AAVhu96 (SEQ ID NO: 3).

FIG. 2F shows a sequence alignment of nucleotides 901 to 1080 of AAV capsids of clade F: AAV9 (SEQ ID NO: 5); AAVhu68 (SEQ ID NO: 7); AAVhu68 engineered (SEQ ID NO: 8); AAVhu95 (SEQ ID NO: 10); AAVhu96 (SEQ ID NO: 11); AAVhu95 engineered (SEQ ID NO: 1); AAVhu96 (SEQ ID NO: 3).

FIG. 2G shows a sequence alignment of nucleotides 1081 to 1260 of AAV capsids of clade F: AAV9 (SEQ ID NO: 5); AAVhu68 (SEQ ID NO: 7); AAVhu68 engineered (SEQ ID NO: 8); AAVhu95 (SEQ ID NO: 10); AAVhu96 (SEQ ID NO: 11); AAVhu95 engineered (SEQ ID NO: 1); AAVhu96 (SEQ ID NO: 3).

FIG. 2H shows a sequence alignment of nucleotides 1261 to 1440 of AAV capsids of clade F: AAV9 (SEQ ID NO: 5); AAVhu68 (SEQ ID NO: 7); AAVhu68 engineered (SEQ ID NO: 8); AAVhu95 (SEQ ID NO: 10); AAVhu96 (SEQ ID NO: 11); AAVhu95 engineered (SEQ ID NO: 1); AAVhu96 (SEQ ID NO: 3).

FIG. 21 shows a sequence alignment of nucleotides 1441 to 1620 of AAV capsids of clade F: AAV9 (SEQ ID NO: 5); AAVhu68 (SEQ ID NO: 7); AAVhu68 engineered (SEQ ID NO: 8); AAVhu95 (SEQ ID NO: 10); AAVhu96 (SEQ ID NO: 11); AAVhu95 engineered (SEQ ID NO: 1); AAVhu96 (SEQ ID NO: 3).

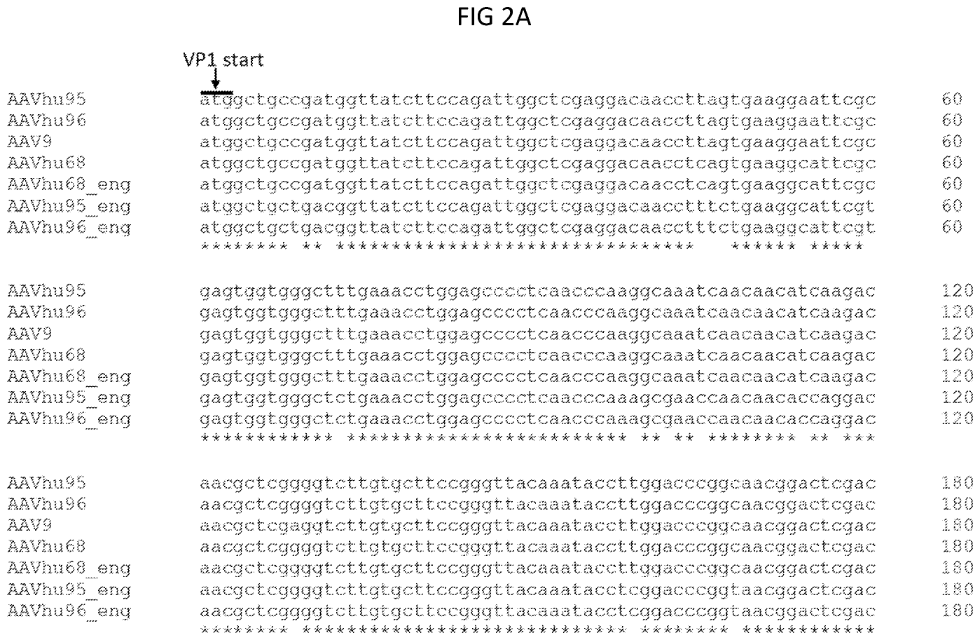

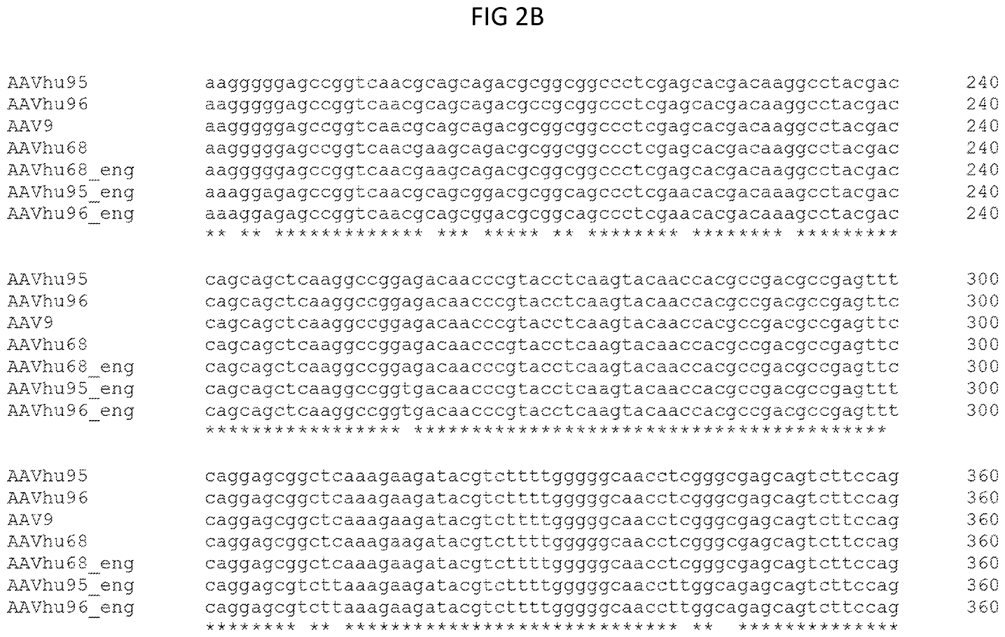

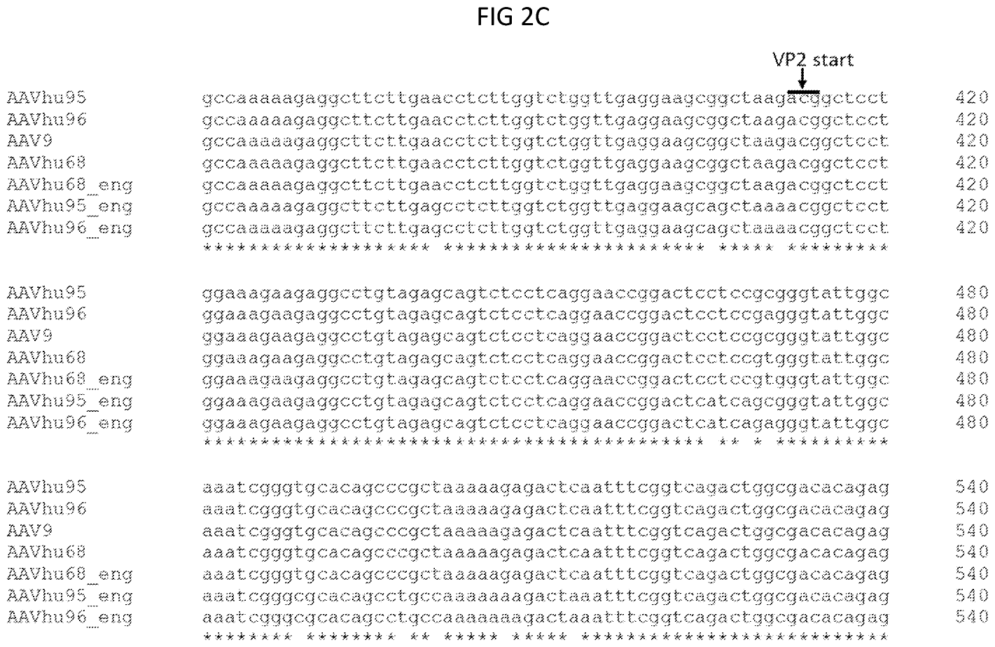

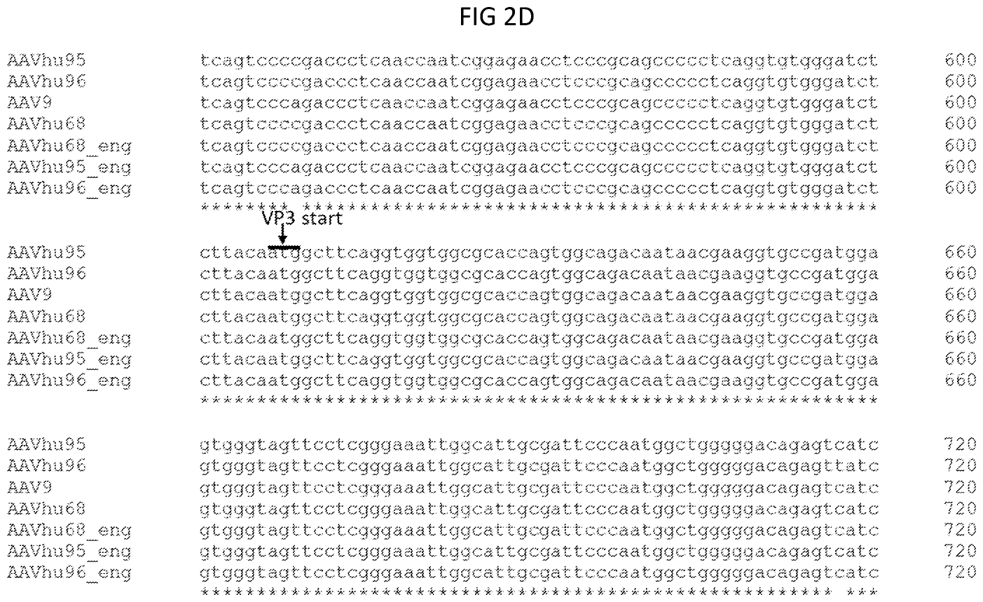

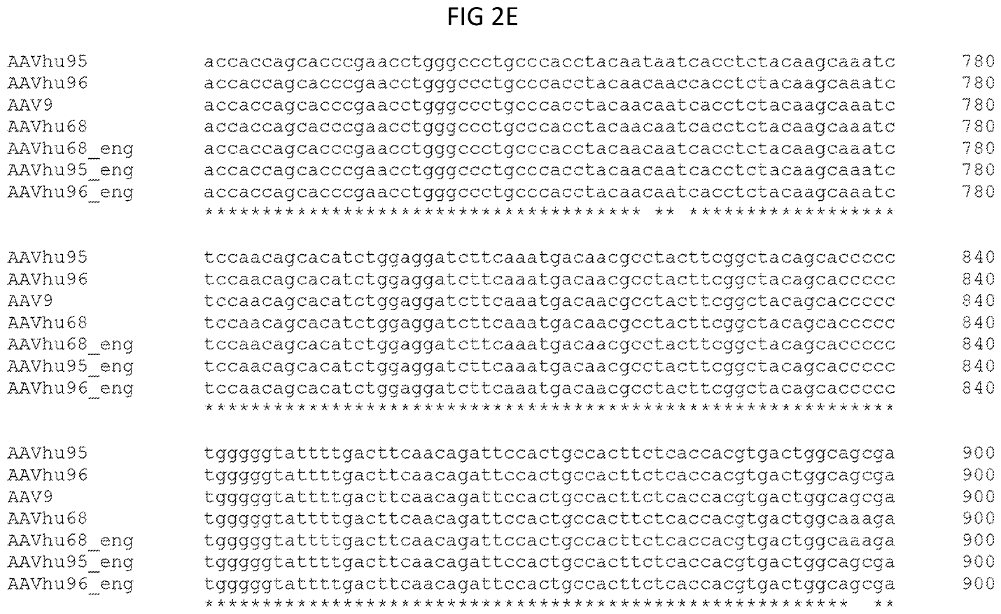

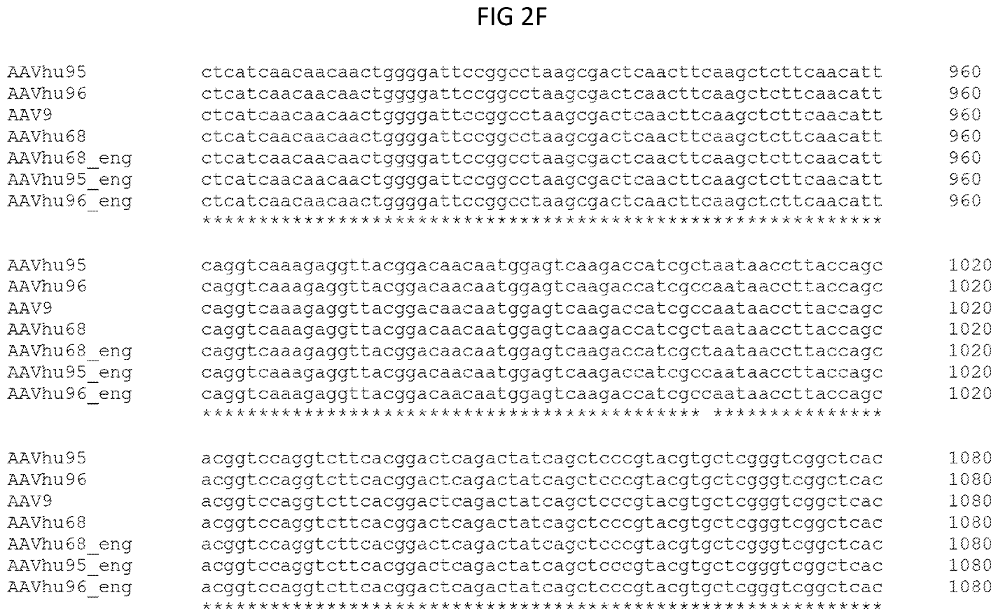

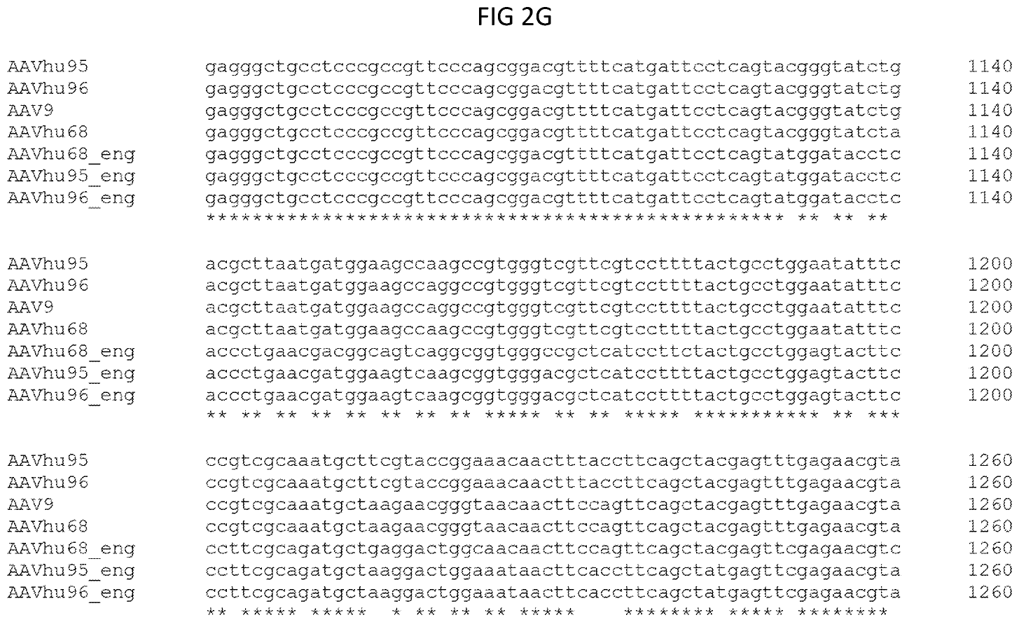

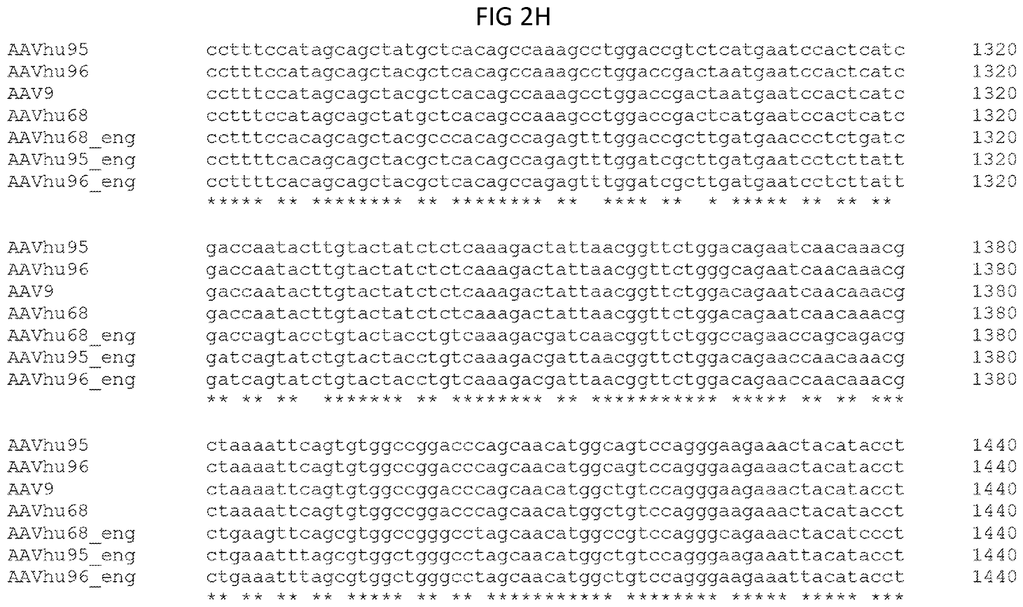

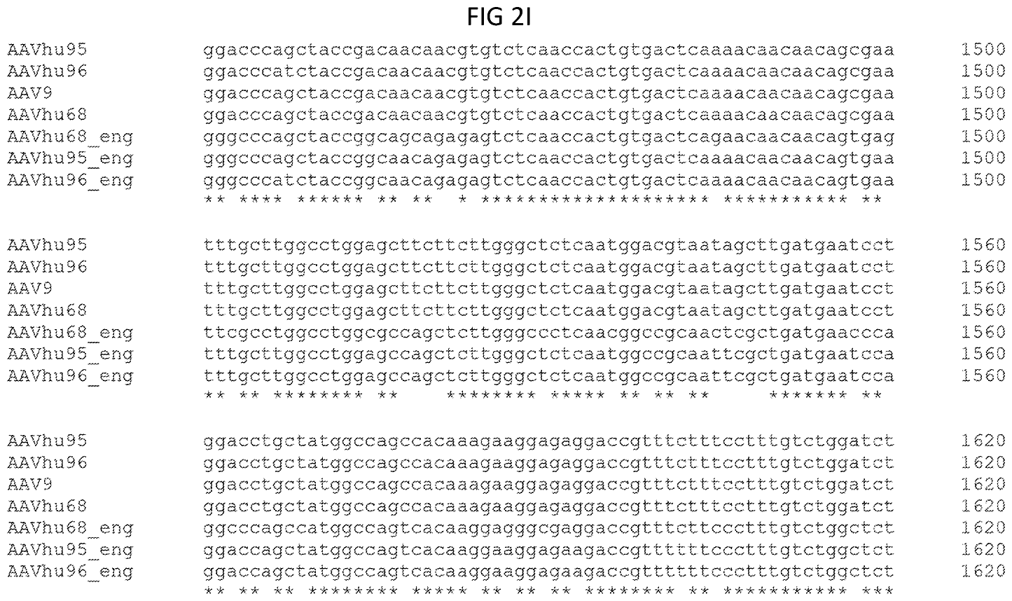

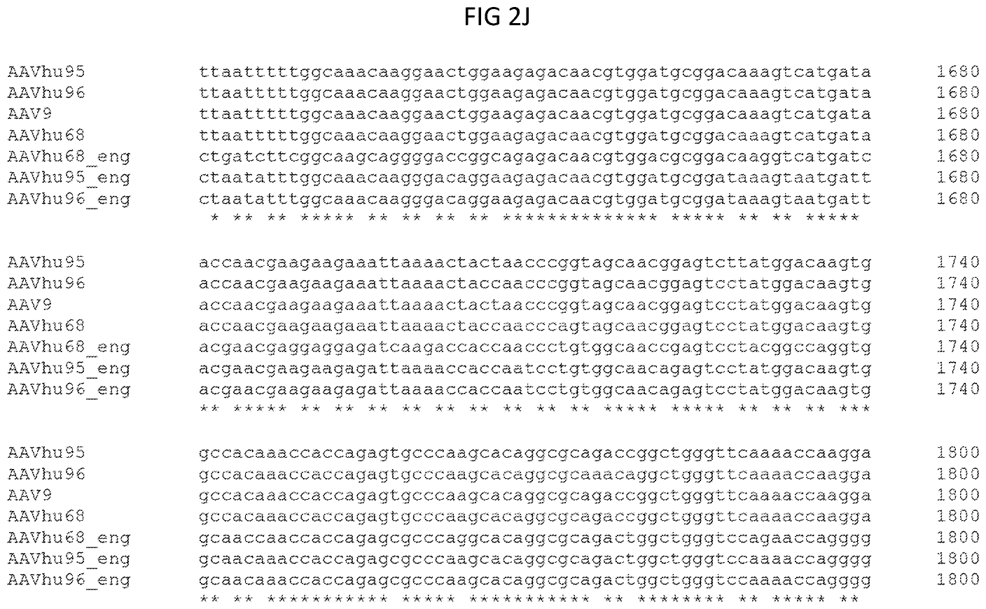

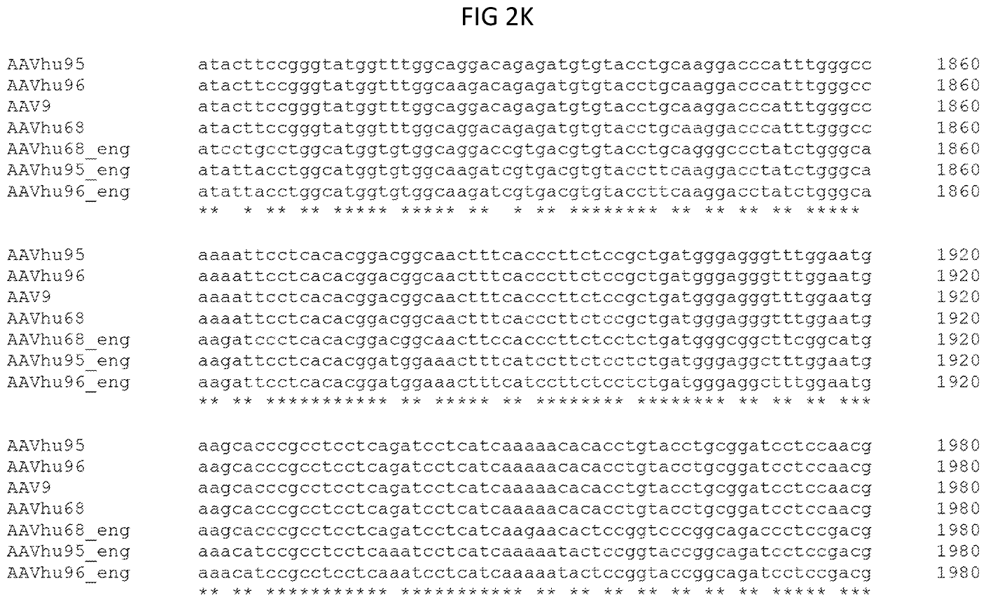

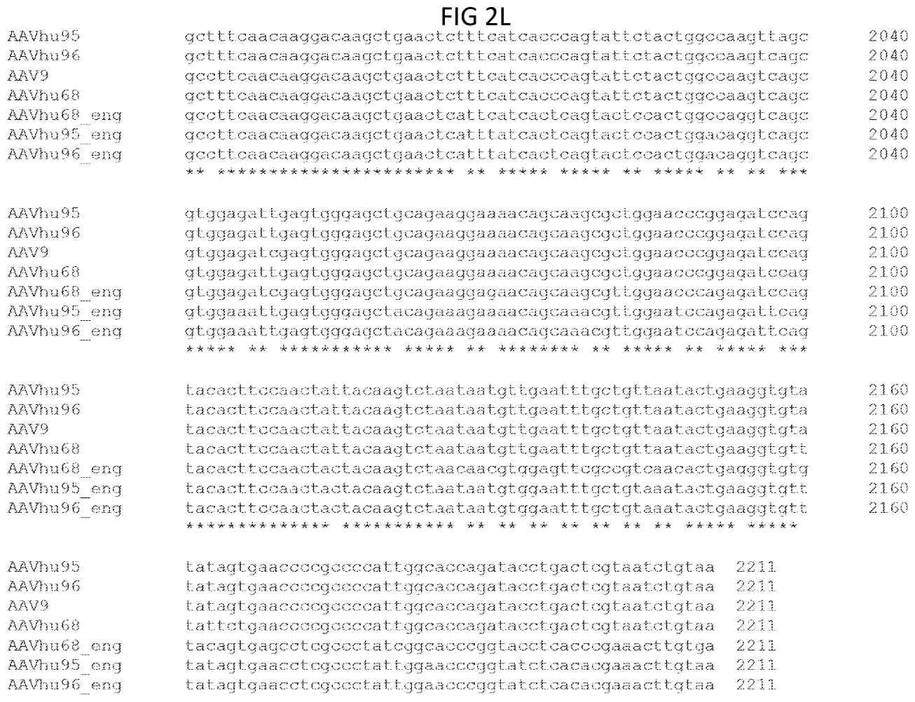

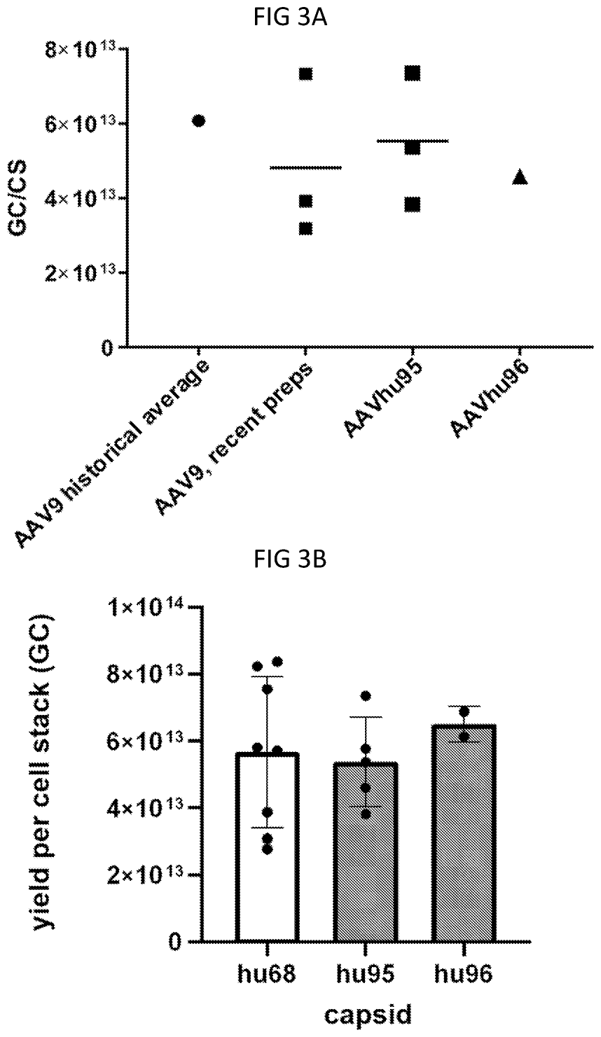

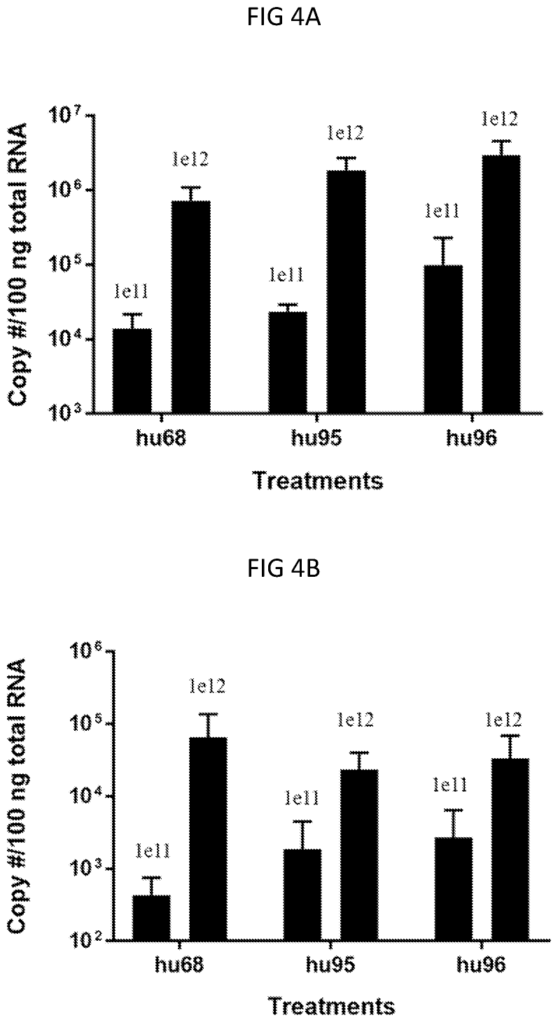

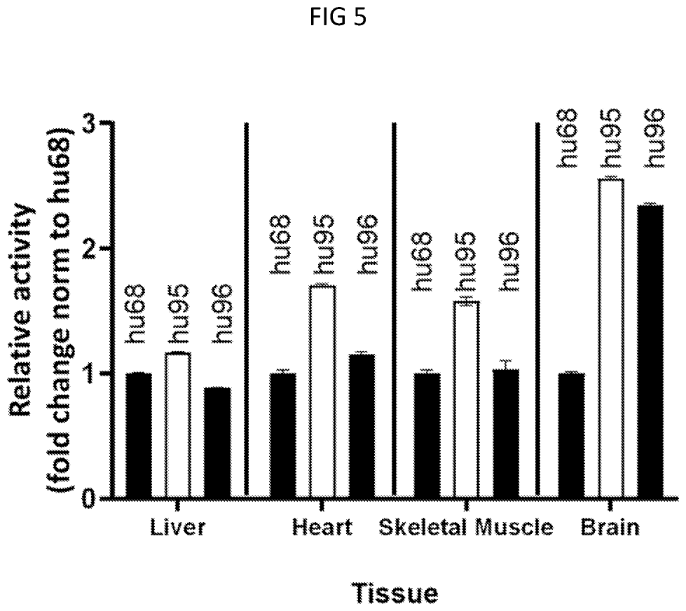

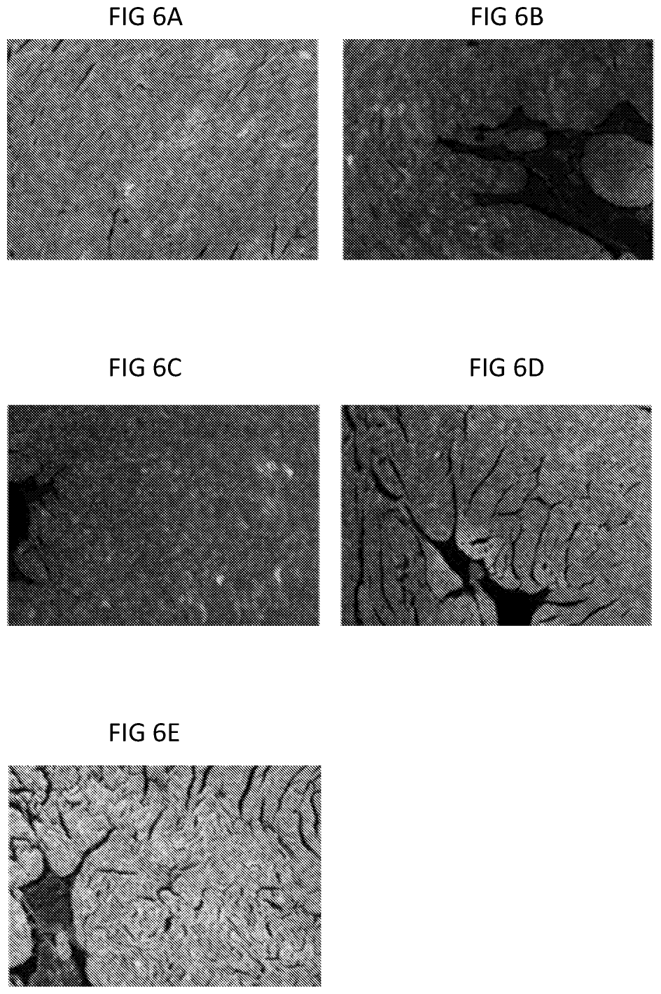

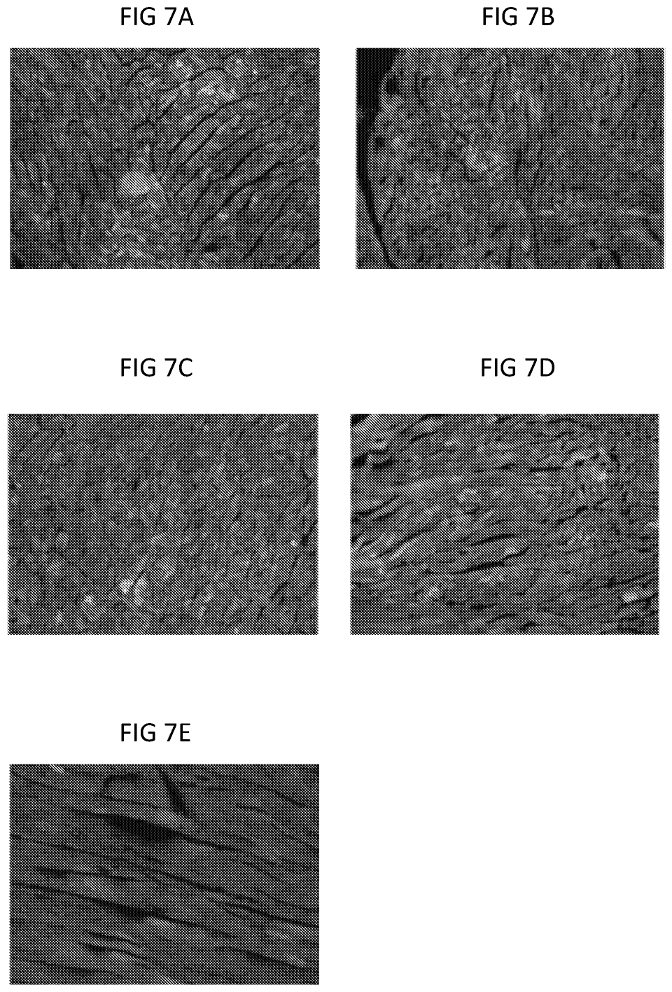

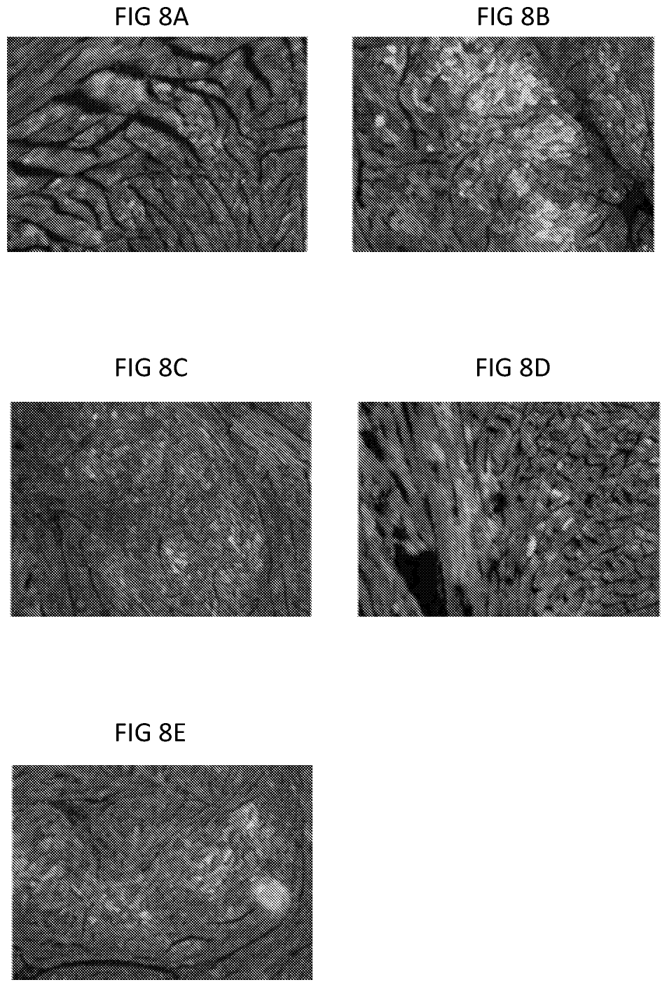

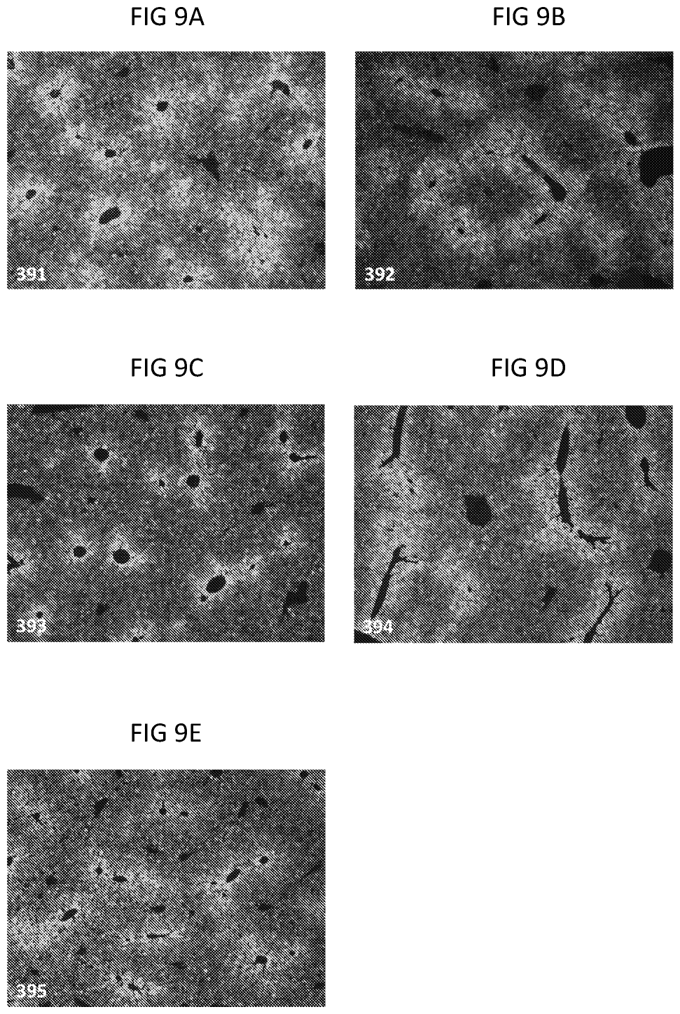

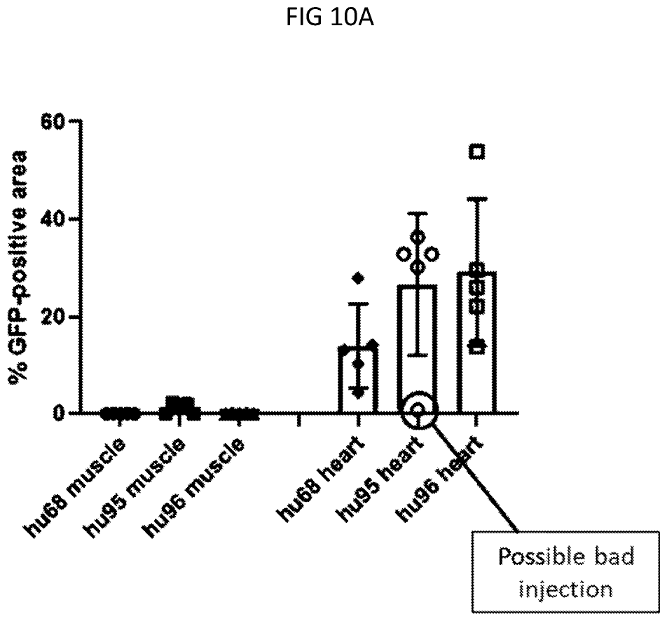

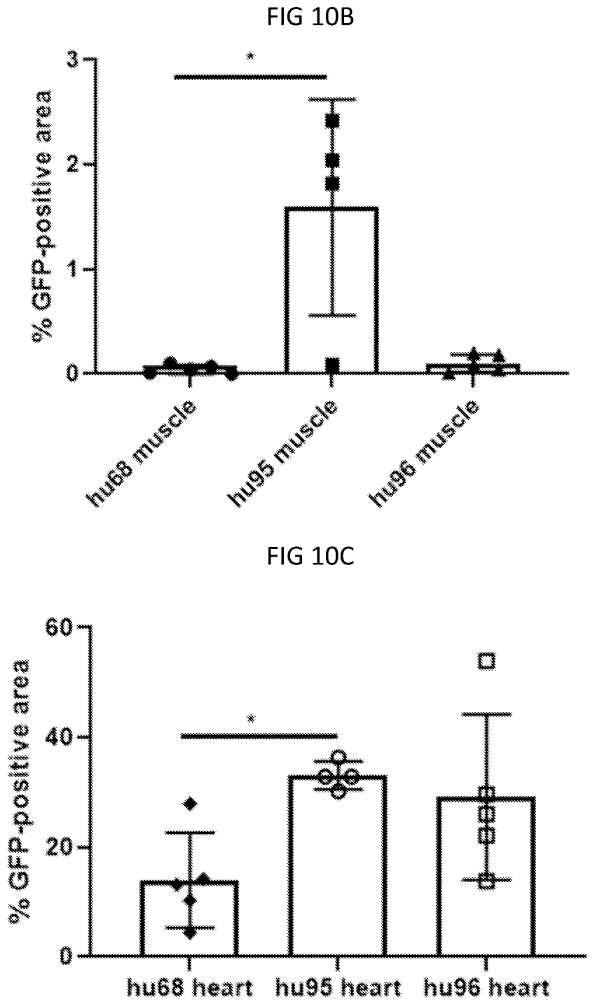

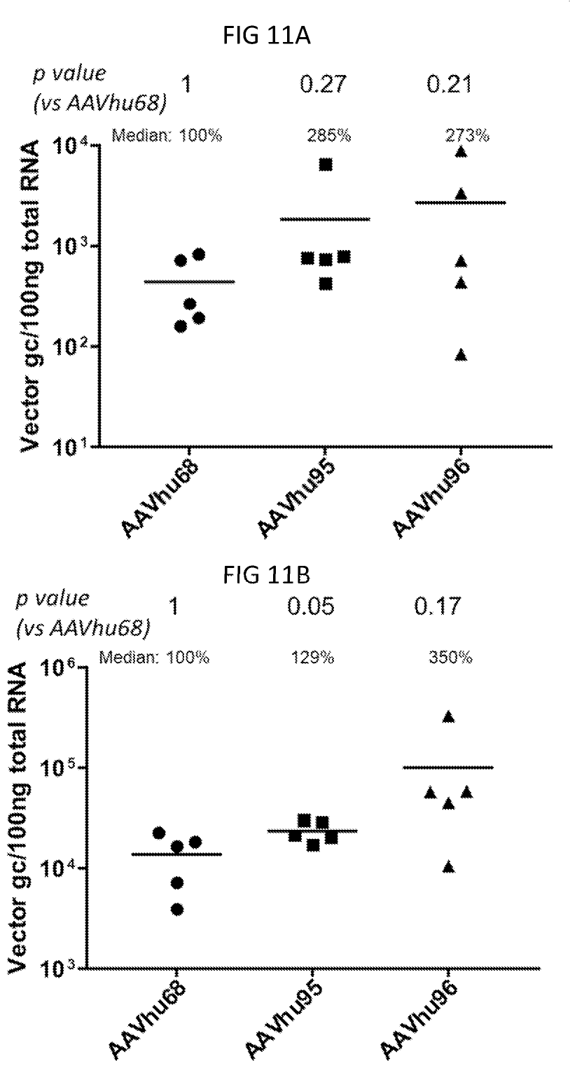

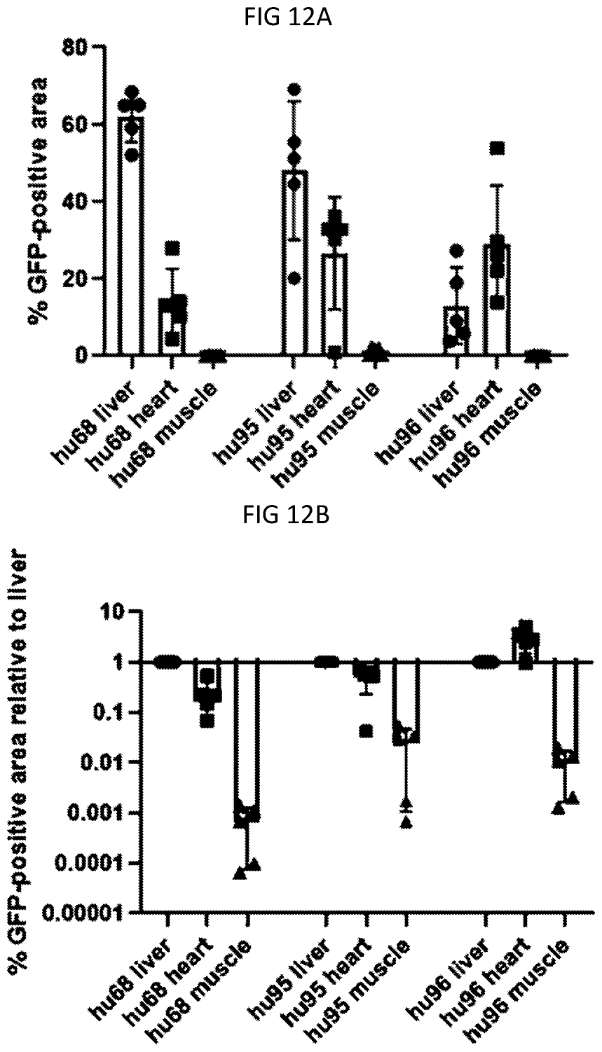

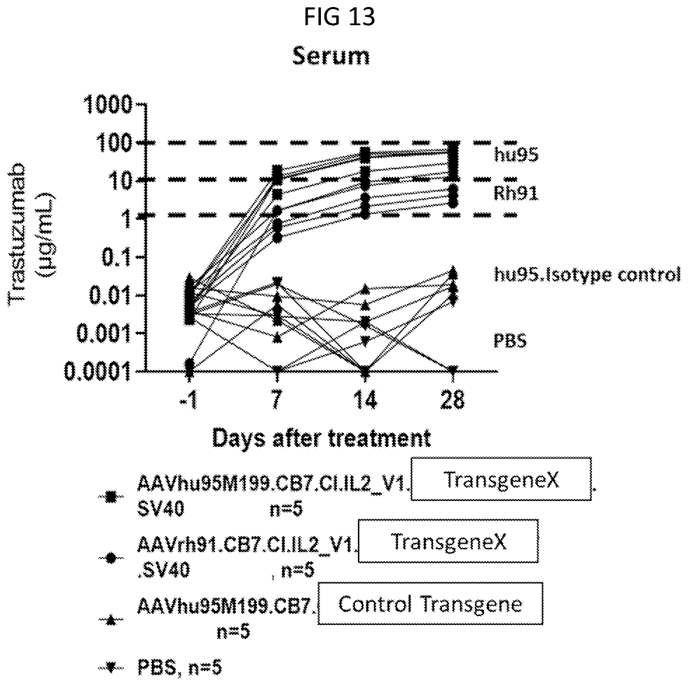

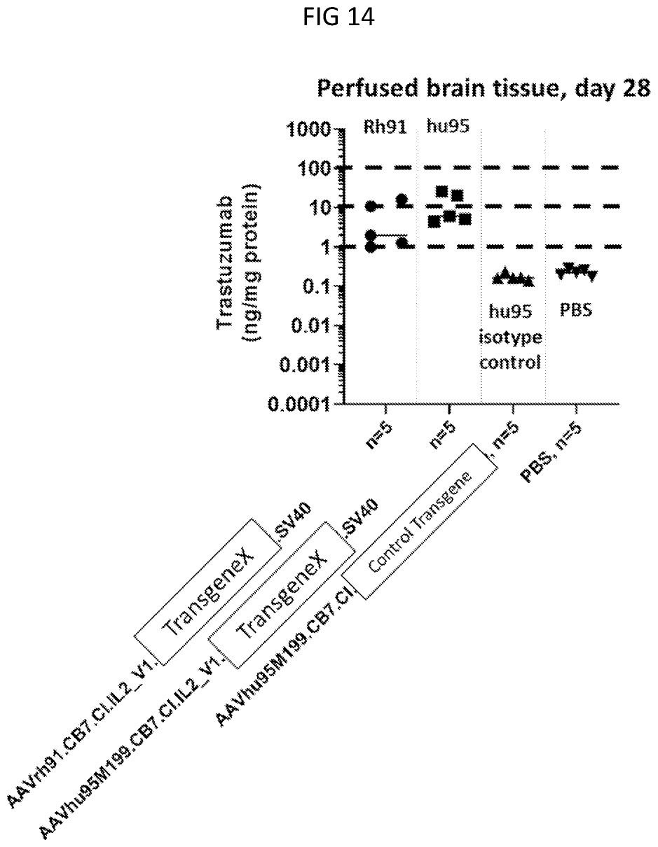

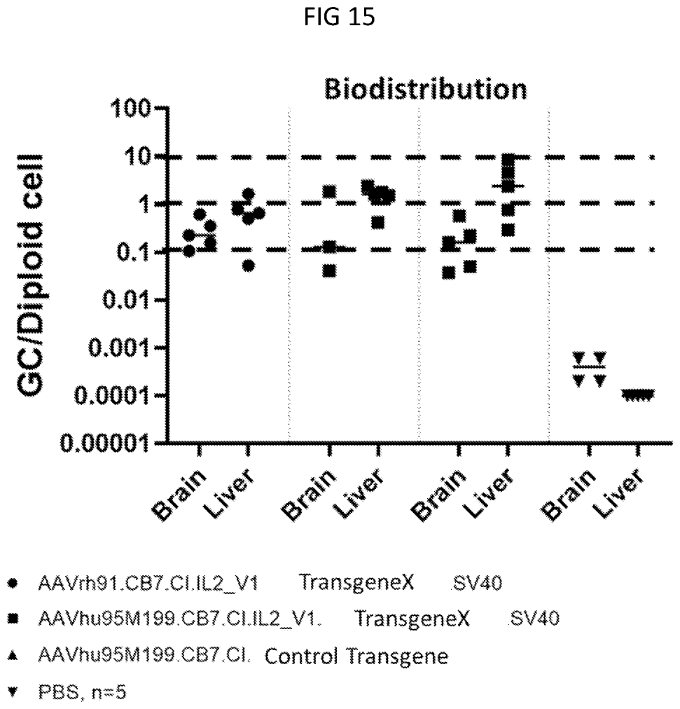

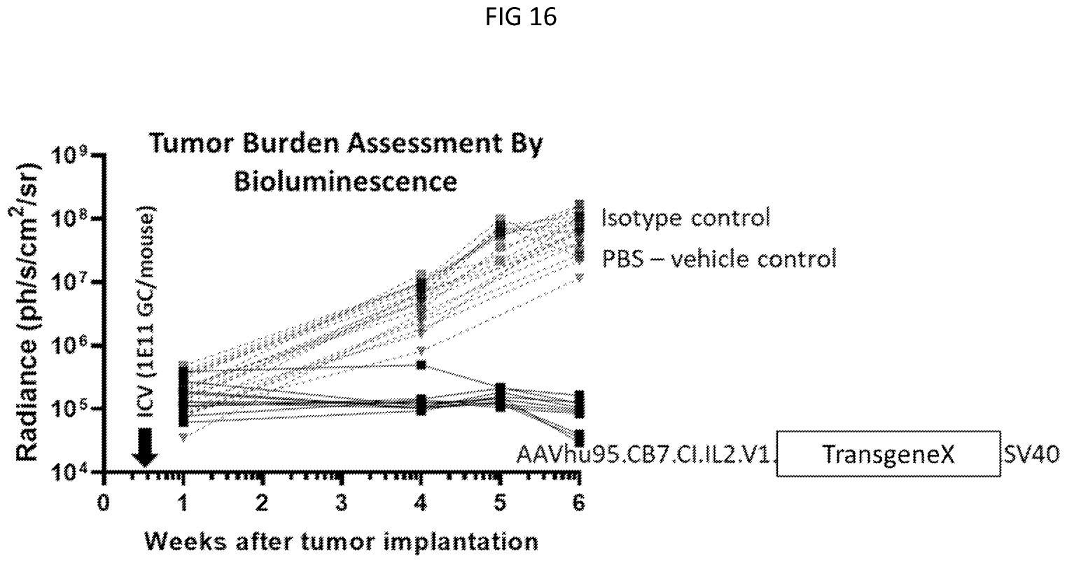

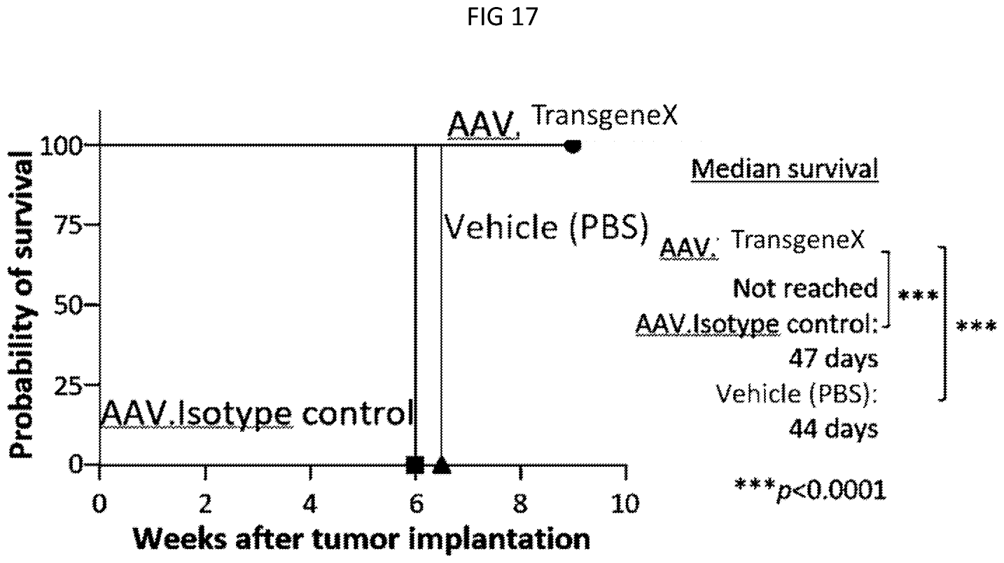

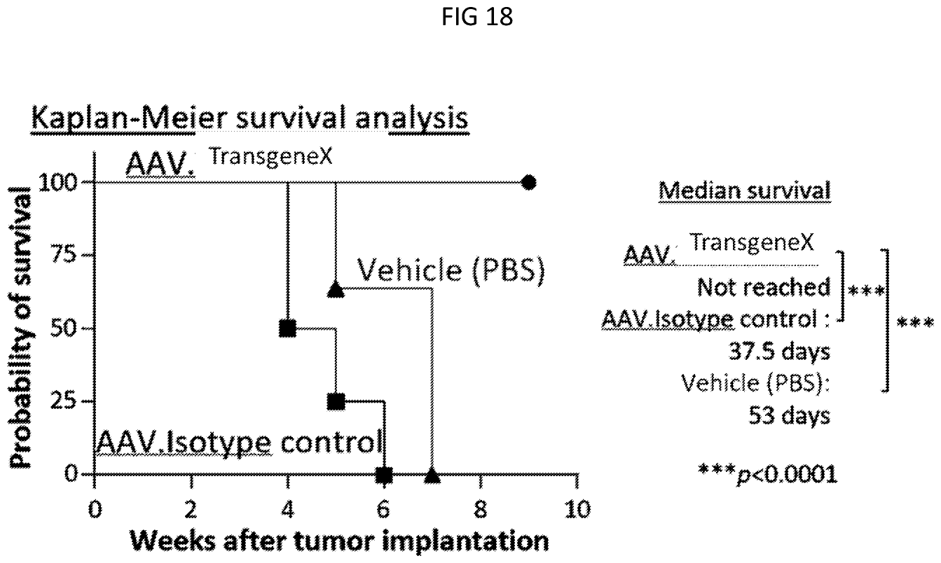

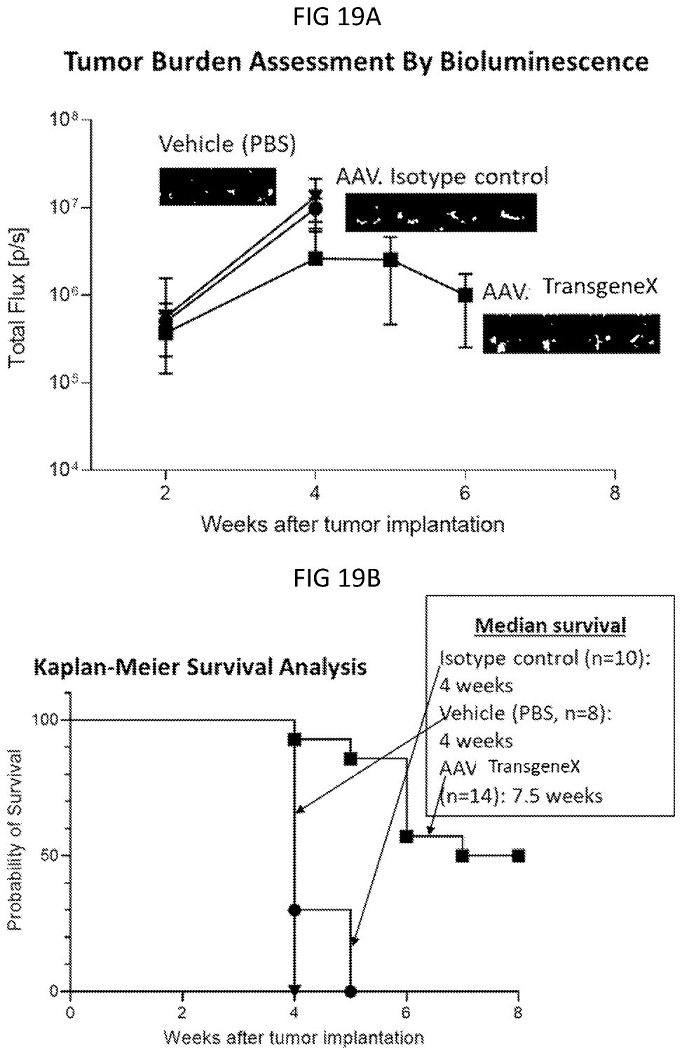

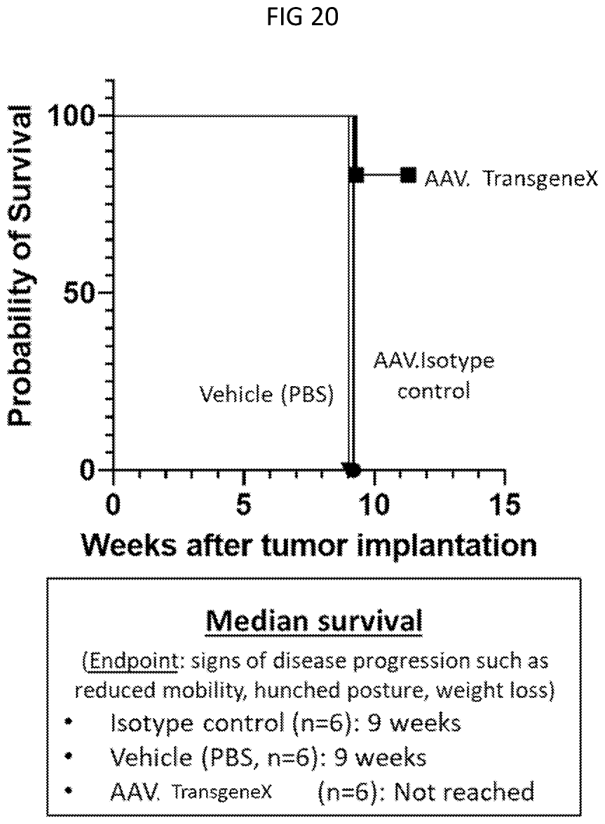

FIG. 2J shows a sequence alignment of nucleotides 1621 to of 1800 capsids of clade F: AAV9 (SEQ ID NO: 5); AAVhu68 (SEQ ID NO: 7); AAVhu68 engineered (SEQ ID NO: 8); AAVhu95 (SEQ ID NO: 10); AAVhu96 (SEQ ID NO: 11); AAVhu95 engineered (SEQ ID NO: 1); AAVhu96 (SEQ ID NO: 3).

FIG. 2K shows a sequence alignment of nucleotides 1801 to 1980 of AAV capsids of clade F: AAV9 (SEQ ID NO: 5); AAVhu68 (SEQ ID NO: 7); AAVhu68 engineered (SEQ ID NO: 8); AAVhu95 (SEQ ID NO: 10); AAVhu96 (SEQ ID NO: 11); AAVhu95 engineered (SEQ ID NO: 1); AAVhu96 (SEQ ID NO: 3).

FIG. 2L shows a sequence alignment of nucleotides 1981 to 2211 of AAV capsids of clade F: AAV9 (SEQ ID NO: 5); AAVhu68 (SEQ ID NO: 7); AAVhu68 engineered (SEQ ID NO: 8); AAVhu95 (SEQ ID NO: 10); AAVhu96 (SEQ ID NO: 11); AAVhu95 engineered (SEQ ID NO: 1); AAVhu96 (SEQ ID NO: 3).

FIG. 3A shows analysis of vector production for AAVhu95 and AAVhu96 from a one CellSTACK® cell culture vessel (Corning®) as compared with AAV9 vector yields from historical average and recent preps, plotted as GC/CS (Genome copies per CellSTACK cell culture vessel (Corning®)).

FIG. 3B shows analysis of vector production for AAVhu95 and AAVhu96 from a one CellSTACK® cell culture vessel (Corning®) as compared with AAVhu68 vector yields from historical average and recent preps, plotted as GC/CS (Genome copies per CellSTACK®cell culture vessel (Corning®)).

FIG. 4A shows eGFP gene expression with AAVhu95 and AAVhu96 in mouse heart tissue 14 days post-injection, as compared with AAVhu68. Mice (n=5) were administered IV with either 1×10¹¹ GC/animal or 1×10¹² GC/animal of AAVhu95.CB7.eGFP, AAVhu96.CB7.eGFP, or AAVhu68.CB7.eGFP. Mice were necropsied at day 14 post-vector administration; RNA were extracted, and vector-derived sequences were quantified by quantitative reverse transcription PCR (RT-qPCR) (copy number/100 ng of total RNA).

FIG. 4B shows eGFP gene expression with AAVhu95 and AAVhu96 in mouse muscle tissue 14 days post-injection, as compared with AAVhu68. Mice (n=5) were administered IV with either 1×10¹¹ GC/animal or 1×10¹² GC/animal of AAVhu95.CB7.eGFP, AAVhu96.CB7.eGFP, or AAVhu68.CB7.eGFP. Mice were necropsied at day 14 post-vector administration; RNA were extracted, and vector-derived sequences were quantified by quantitative reverse transcription PCR (RT-qPCR) (copy number/100 ng of total RNA).

FIG. 5 shows high result of the high dose barcode study of the novel clade F capsids (AAVhu95 and AAVhu96) in comparison to AAVhu68, after IV and ICM administration in NHP at a dose of 2.5×10¹³ GC/kg of each vector (total 7.5×10¹³ GC/kg). NHP were necropsied and liver, heart, skeletal muscle and brain tissue were analyzed and plotted as for relative activity (fold change normal to AAVhu68 signal).

FIG. 6A shows a representative image of heart muscle tissue from GFP expression microscopy analysis following IV delivery of AAVhu68 (CB7.eGFP.WPRE.rBG) vector in mice at a dose of 1×10¹ GC.

FIG. 6B shows another representative image of heart muscle tissue from GFP expression microscopy analysis following IV delivery of AAVhu68 (CB7.eGFP.WPRE.rBG) vector in mice at a dose of 1×10¹² GC.

FIG. 6C shows yet another representative image of heart muscle tissue from GFP expression microscopy analysis following IV delivery of AAVhu68 (CB7.eGFP.WPRE.rBG) vector in mice at a dose of 1×10¹² GC.

FIG. 6D shows yet another representative image of heart muscle tissue from GFP expression microscopy analysis following IV delivery of AAVhu68 (CB7.eGFP.WPRE.rBG) vector in mice at a dose of 1×10¹² GC.

FIG. 6E shows yet another representative image of heart muscle tissue from GFP expression microscopy analysis following IV delivery of AAVhu68 (CB7.eGFP.WPRE.rBG) vector in mice at a dose of 1×10¹² GC.

FIG. 7A shows a representative image of heart muscle tissue from GFP expression microscopy analysis following IV delivery of AAVhu95 (CB7.eGFP.WPRE.rBG) vector in mice at a dose of 1×10¹² GC.

FIG. 7B shows another representative image of heart muscle tissue from GFP expression microscopy analysis following IV delivery of AAVhu95 (CB7.eGFP.WPRE.rBG) vector in mice at a dose of 1×10¹² GC.

FIG. 7C shows yet another representative image of heart muscle tissue from GFP expression microscopy analysis following IV delivery of AAVhu95 (CB7.eGFP.WPRE.rBG) vector in mice at a dose of 1×10¹² GC.

FIG. 7D shows yet another representative image of heart muscle tissue from GFP expression microscopy analysis following IV delivery of AAVhu95 (CB7.eGFP.WPRE.rBG) vector in mice at a dose of 1×10¹² GC.

FIG. 7E shows yet another representative image of heart muscle tissue from GFP expression microscopy analysis following IV delivery of AAVhu95 (CB7.eGFP.WPRE.rBG) vector in mice at a dose of 1×10¹² GC.

FIG. 8A shows a representative image of heart muscle tissue from GFP expression microscopy analysis following IV delivery of AAVhu96 (CB7.eGFP.WPRE.rBG) vector in mice at a dose of 1×10¹² GC.

FIG. 8B shows another representative image of heart muscle tissue from GFP expression microscopy analysis following IV delivery of AAVhu96 (CB7.eGFP.WPRE.rBG) vector in mice at a dose of 1×10¹² GC.

FIG. 8C shows yet another representative image of heart muscle tissue from GFP expression microscopy analysis following IV delivery of AAVhu96 (CB7.eGFP.WPRE.rBG) vector in mice at a dose of 1×10¹² GC.

FIG. 8D shows yet another representative image of heart muscle tissue from GFP expression microscopy analysis following IV delivery of AAVhu96 (CB7.eGFP.WPRE.rBG) vector in mice at a dose of 1×10¹² GC.

FIG. 8E shows yet another representative image of heart muscle tissue from GFP expression microscopy analysis following IV delivery of AAVhu96 (CB7.eGFP.WPRE.rBG) vector in mice at a dose of 1×10¹² GC.

FIG. 9A shows a representative image of liver tissue from GFP expression microscopy analysis following IV delivery of AAVhu95 (CB7.eGFP.WPRE.rBG) vector in mice at a dose of 1×10¹² GC.

FIG. 9B shows another representative image of liver tissue from GFP expression microscopy analysis following IV delivery of AAVhu95 (CB7.eGFP.WPRE.rBG) vector in mice at a dose of 1×10¹² GC.

FIG. 9C shows yet another representative image of liver tissue from GFP expression microscopy analysis following IV delivery of AAVhu95 (CB7.eGFP.WPRE.rBG) vector in mice at a dose of 1×10¹² GC.

FIG. 9D shows yet another representative image of liver tissue from GFP expression microscopy analysis following IV delivery of AAVhu95 (CB7.eGFP.WPRE.rBG) vector in mice at a dose of 1×10¹² GC.

FIG. 9E shows yet another representative image of liver tissue from GFP expression microscopy analysis following IV delivery of AAVhu95 (CB7.eGFP.WPRE.rBG) vector in mice at a dose of 1×10¹² GC.

FIG. 10A shows percent GFP-positive area in samples of analyzed tissues of heart and muscle from mice that were administered with AAVhu68.GFP, AAVhu95.GFP, or AAVhu96.GFP at a dose of 1×10¹² GC/animal.

FIG. 10B shows percent GFP-positive area in samples of analyzed muscle tissue from mice that were administered with AAVhu68.GFP, AAVhu95.GFP, or AAVhu96.GFP at a dose of 1×10¹² GC/animal, wherein the data has been analyzed without including a data point attributed to a possible bad injection.

FIG. 10C shows percent GFP-positive area in samples of analyzed heart tissue from mice that were administered with AAVhu68.GFP, AAVhu95.GFP, or AAVhu96.GFP at a dose of 1×10¹² GC/animal, wherein the data has been analyzed without including a data point attributed to a possible bad injection.

FIG. 11A shows analysis of the qualitative expression of a reporter gene in skeletal muscle tissue 14 days-post injection, as analyzed by RT-qPCR in tissue following an administration with a dose of 1×10¹¹ GC. FIG.

11B shows analysis of the qualitative expression of a reporter gene in heart tissue 14 days-post injection, as analyzed by RT-qPCR in tissue following an administration with a dose of 1×10¹¹ GC.

FIG. 12A shows percent GFP-positive area in samples of analyzed of tissues liver, heart and muscle from mice that were administered with AAVhu68.GFP, AAVhu95.GFP, or AAVhu96.GFP at a dose of 1×10¹² GC/animal.

FIG. 12B shows percent GFP-positive area relative to liver in sample of analyzed of tissues liver, heart and muscle from mice that were administered with AAVhu68.GFP, AAVhu95.GFP, or AAVhu96.GFP at a dose of 1×10¹² GC/animal.

FIG. 13 shows expression levels of expressed ProteinX encoded by TransgeneX (μg/mL) as measured in serum samples at day—1, 7, 14, and 28 post administration with AAVhu95.CB.CI.IL2_V1.TransgeneX.SV40, AAVrh91.CB.CI.IL2_V1. TransgeneX.SV40 in comparison with capsid control and PBS.

FIG. 14 shows expression levels of expressed ProteinX encoded by TransgeneX (μg/mL) as measured in brain tissue samples at day—1, 7, 14, and 28 post administration with AAVhu95.CB.CI.IL2_V1.TransgeneX.SV40, AAVrh91.CB.CI.IL2_V1.TransgeneX.SV40 in comparison with capsid control and PBS.

FIG. 15 shows vector biodistribution (GC/diploid cell) samples at day—1, 7, 14, and 28 post administration with AAVhu95.CB.CI.IL2 V1.TransgeneX.SV40, AAVrh91.CB.CI.IL2_V1. TransgeneX.SV40 in comparison with capsid control and PBS.

FIG. 16 shows quantified results of the tumor bioluminescence assessment in mice xenograft (MDA-MB-453 (ER−/PR−/HER2+)) post treatment with AAVhu95.CB.CI.IL2.V1.TransgeneX in comparison with isotype control.

FIG. 17 shows Kaplan-Meier survival analysis (disease remission) of probability of survival in tumor bearing mice (MDA-MB-453 (ER−/PR−/HER2+) xenografts) treated with AAVhu95.CB.CI.IL2.V1.TransgeneX.SV40.

FIG. 18 Kaplan-Meier survival analysis (prophylactic treatment) of probability of survival in tumor bearing mice (BT-474 (ER+/PR+/HER2+) brain xenografts) treated with AAVhu95. CB. CI.IL2.V1.TransgeneX.SV40.

FIG. 19A shows quantified results of the tumor bioluminescence assessment in mice xenograft (BT-474 Clone 5 trastuzumab resistant (ER+/PR+/HER2+) xenograft) post treatment with AAVhu95.CB.CI.IL2.V1.TransgeneX.SV40 in comparison with isotype control.

FIG. 19B shows Kaplan-Meier survival analysis (prophylactic treatment) of probability of survival in tumor bearing mice (BT-474 Clone 5 trastuzumab resistant (ER+/PR+/HER2+) xenograft) treated with AAVhu95.CB.CI.IL2.V1.TransgeneX.SV40 FIG. 20 shows Kaplan-Meier survival analysis (prophylactic treatment) of probability of survival in tumor bearing mice (MDA-MB-231HER2/low tumors) treated with AAVhu95. CB. CI.IL2.V1.TransgeneX.SV40.

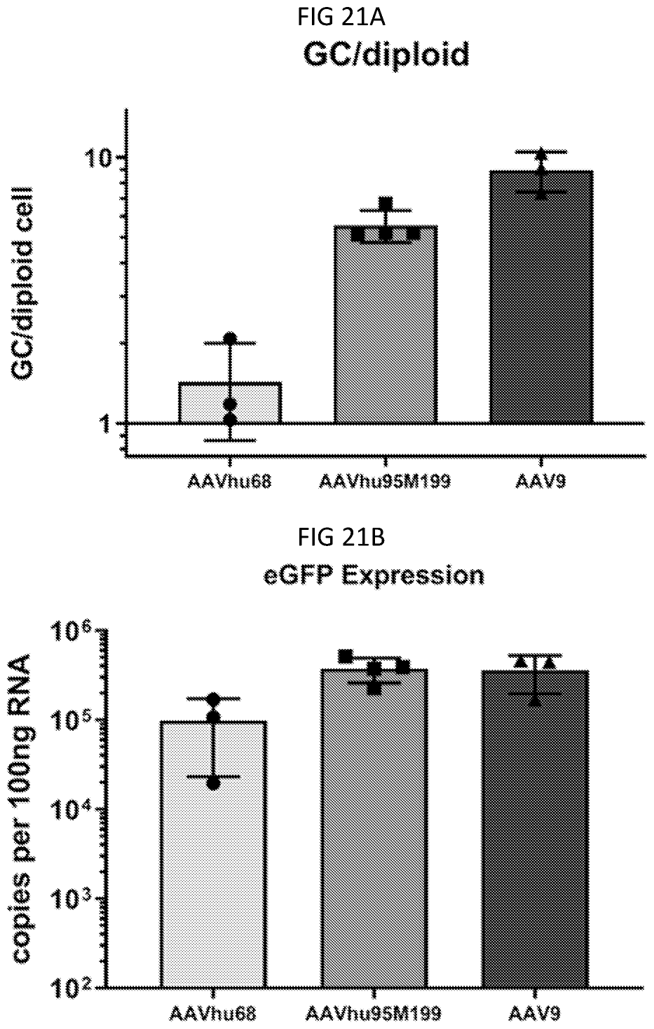

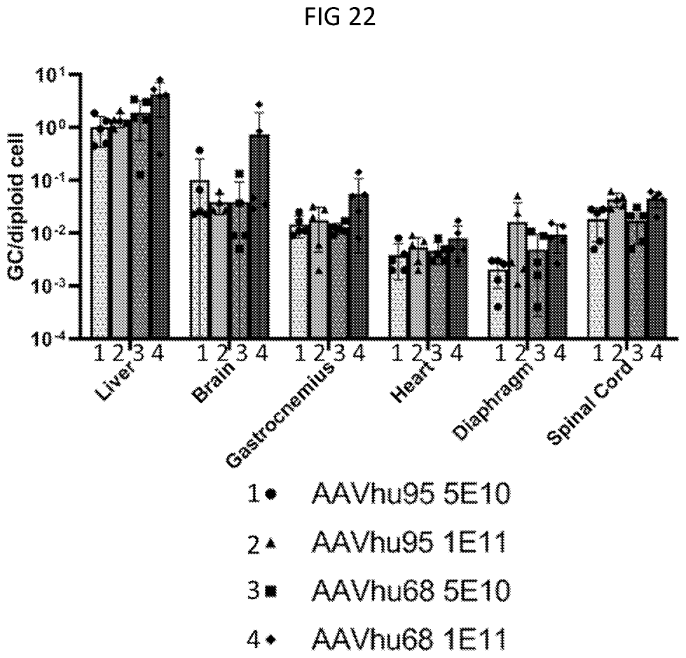

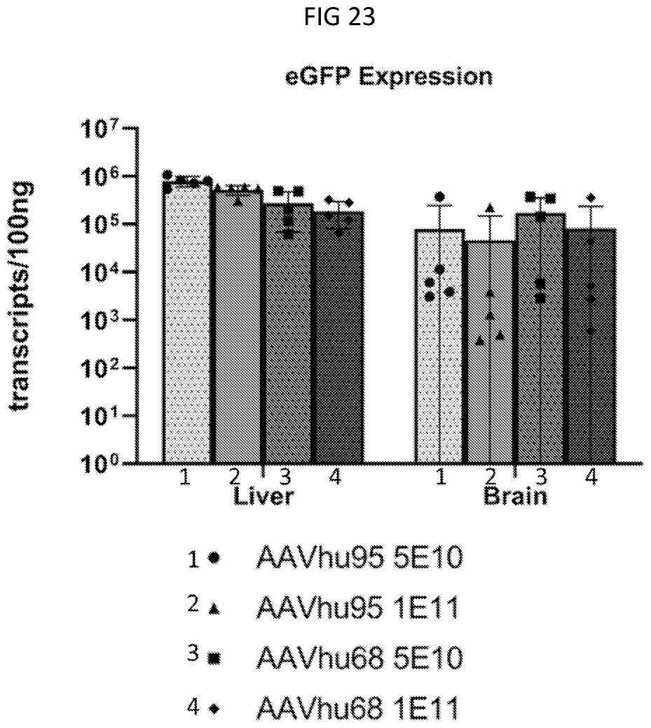

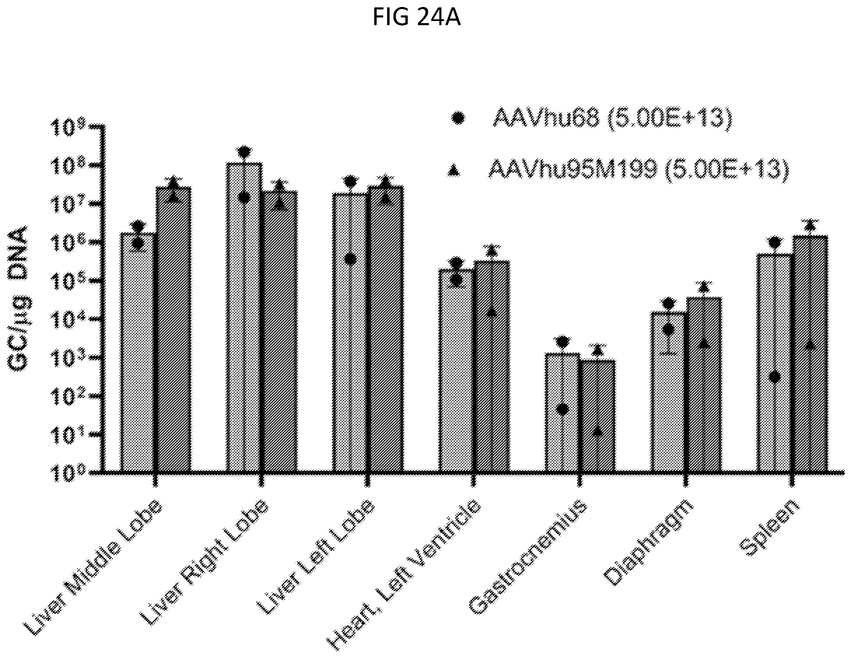

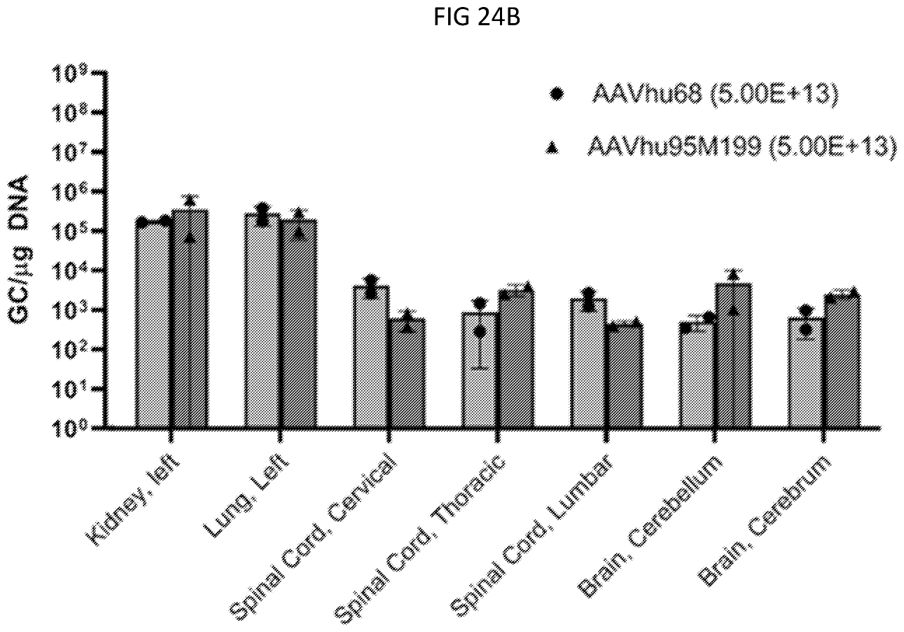

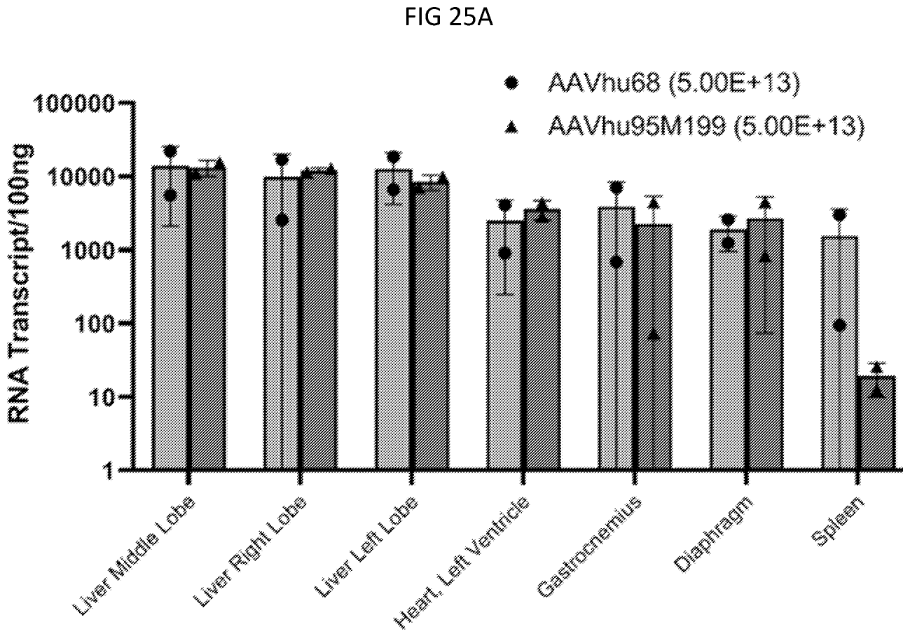

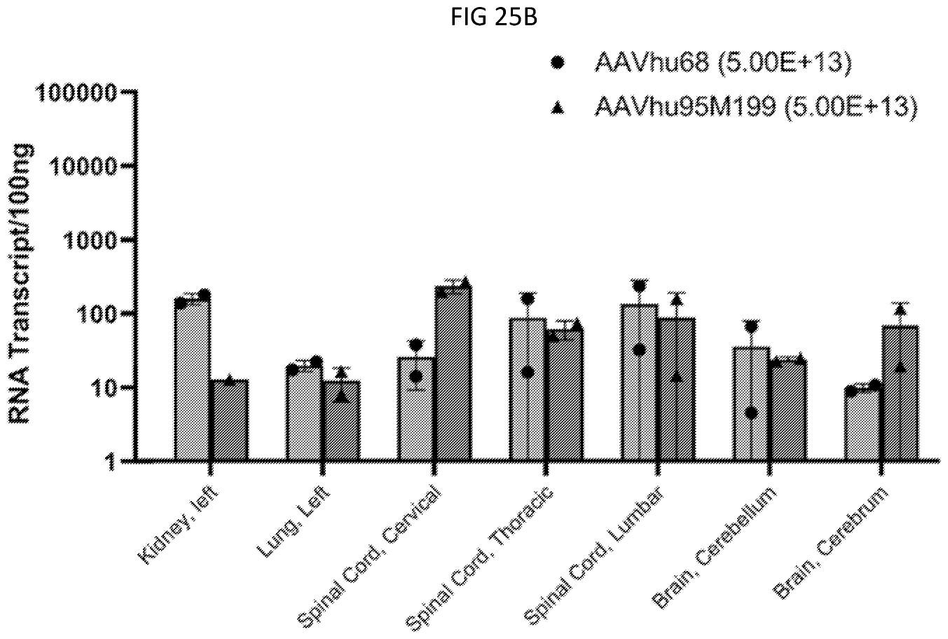

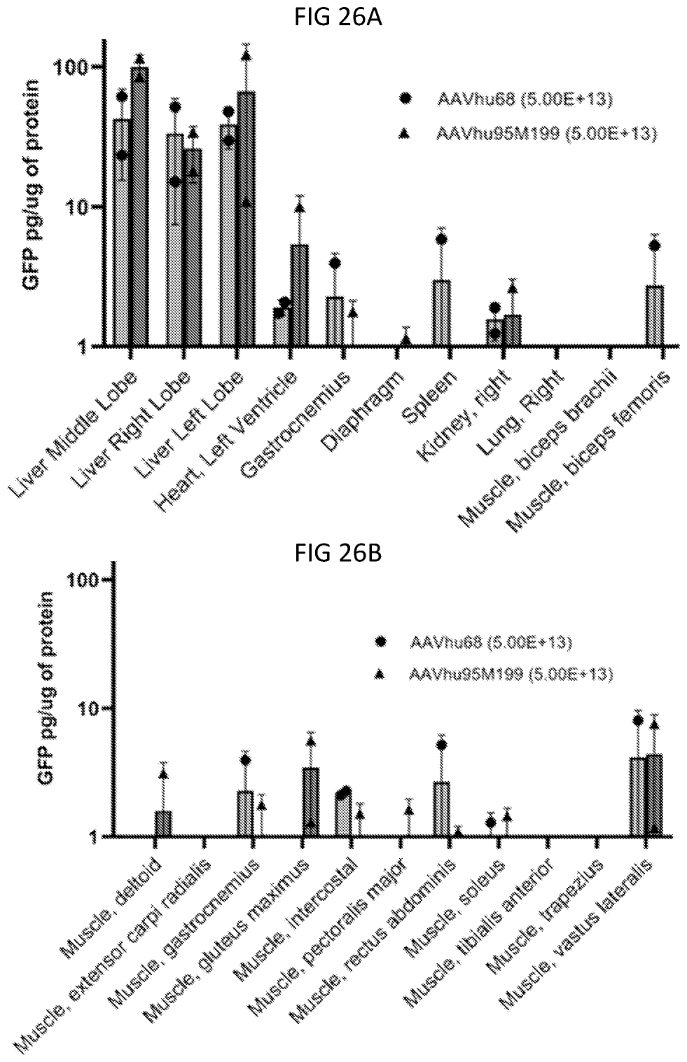

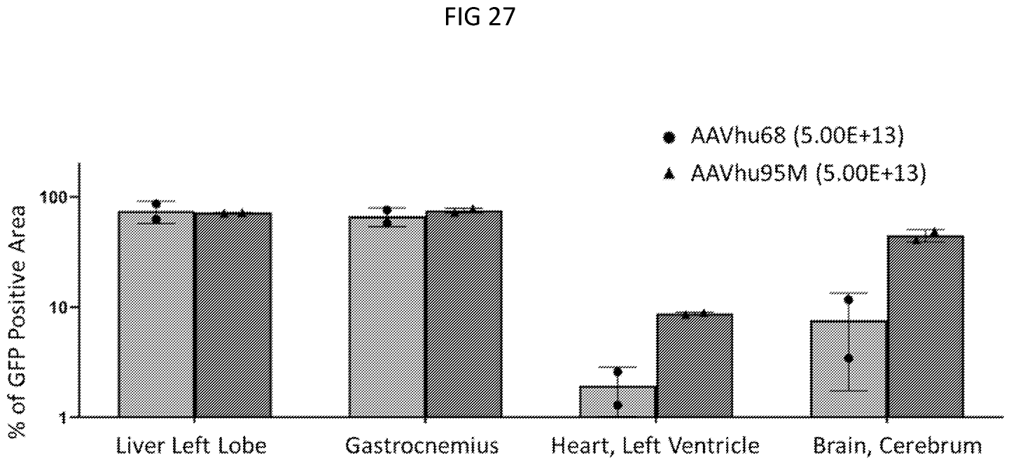

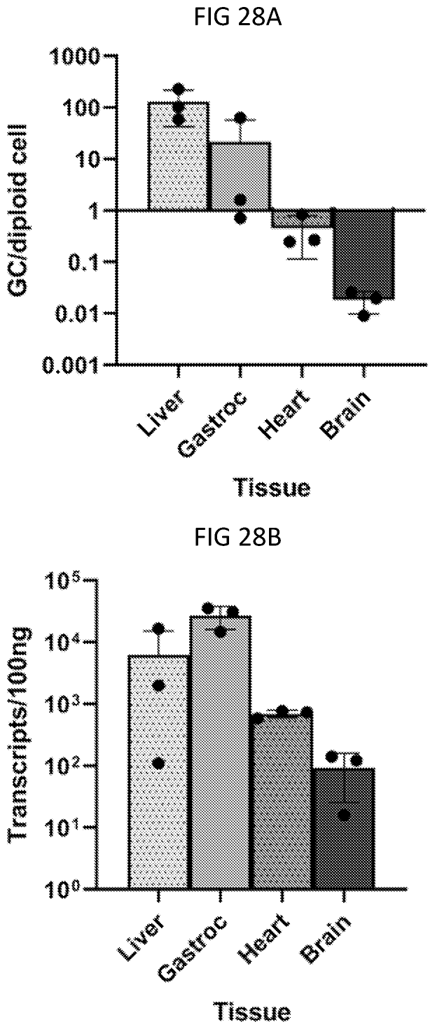

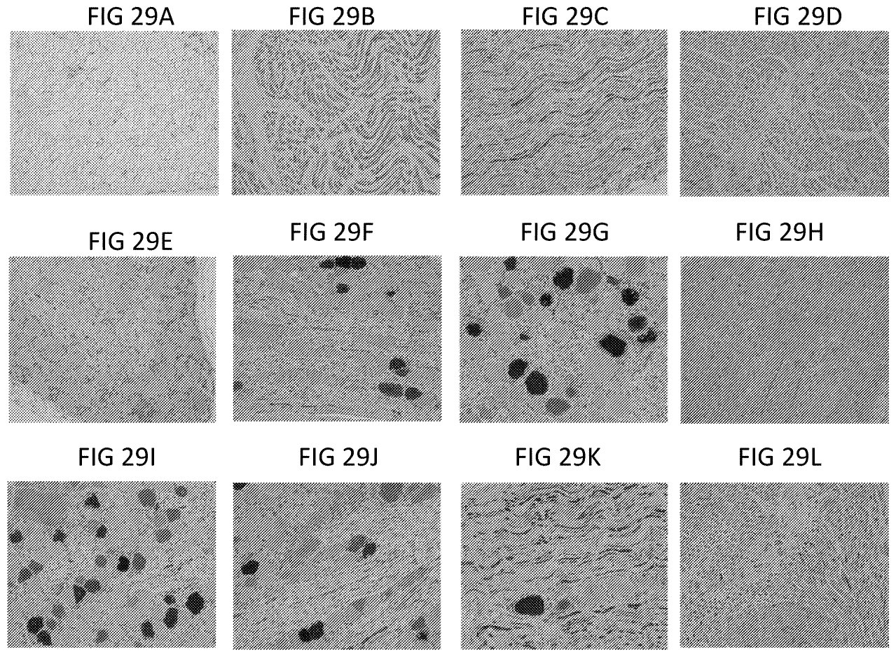

FIG. 21A shows measured copies of AAV vector genomes (DNA), as measured by qPCR from liver tissue samples following intravenous administration of AAVhu68.CB7.CI.eGFP.WPRE.rBG, AAVhu95M199.CB7.CI.eGFP.WPRE.rBG, AAV9.CB7.CI.eGFP.WPRE.rBG, and plotted as genome copies/diploid cell (GC/diploid cell).

FIG. 21B shows transgene expression (RNA) as measured by RT-qPCR from liver tissue samples following intravenous administration of AAVhu68.CB7.CI.eGFP.WPRE.rBG, AAVhu95M199.CB7.CI.eGFP.WPRE.rBG, AAV9.CB7.CI.eGFP.WPRE.rBG, and plotted as transcripts/100 ng total RNA.

FIG. 22 shows measured copies of AAV vector genomes (DNA), as measured by qPCR from tissue (liver, brain, gastrocnemius, heart, diaphragm) samples following ICV administration of AAVhu68.CB7.CI.eGFP.WPRE.rBG, and AAVhu95M199.CB7.CI.eGFP.WPRE.rBG at doses of 5×10¹⁰ (5E10) and 1×10¹¹ (1E11) GC/mouse, and plotted as genome copies/diploid cell (GC/diploid cell). Results of Immunohistochemistry microscopy confirmed results of the eGFP expression (results not shown).

FIG. 23 shows transgene expression (RNA) as measured by RT-qPCR from liver and brain tissue samples following ICV administration of AAVhu68.CB7.CI.eGFP.WPRE.rBG, and AAVhu95M199.CB7.CI.eGFP.WPRE.rBG at doses of 5×10¹⁰ (5E10) and 1×10¹¹ (1E11) GC/mouse, and plotted as transcripts/100 ng total RNA.

FIG. 24A shows Vector DNA (GC/g DNA) biodistribution in harvested tissue samples of major organs (right, middle, and left lobes of liver, left ventricle of a heart, gastroenemius, diaphragm, spleen) following administration of AAVhu68.CB7.CI.eGFP.WPRE.rBG, and AAVhu95M199.CB7.CI.eGFP.WPRE.rBG via IV at a dose of 5×10¹³ (5.00E+13).

FIG. 24B shows Vector DNA (GC/g DNA) biodistribution in harvested tissue samples of major organs (kidney, lung, spinal cord (cervical, thoracic, lumbar) and brain (cerebellum, cerebrum) following administration of AAVhu68.CB7.CI.eGFP.WPRE.rBG, and AAVhu95M199.CB7.CI.eGFP.WPRE.rBG via IV at a dose of 5×10¹³ (5.00E+13) in Cynomologus Macaques.

FIG. 25A shows RNA transcript (RNA transcript/100 ng) biodistribution in harvested tissue samples of major organs (right, middle, and left lobes of liver, left ventricle of a heart, gastrocnemius, diaphragm, spleen) following administration of AAVhu68.CB7.CI.eGFP.WPRE.rBG, and AAVhu95M199.CB7.CI.eGFP.WPRE.rBG via IV at a dose of 5×10¹³ (5.00E+13) in Cynomologus Macaques.

FIG. 25B shows RNA transcript (RNA transcript/100 ng) biodistribution in harvested tissue samples of major organs (kidney, lung, spinal cord (cervical, thoracic, lumbar) and brain (cerebellum, cerebrum) following administration of AAVhu68.CB7.CI.eGFP.WPRE.rBG, and AAVhu95M199.CB7.CI.eGFP.WPRE.rBG via IV at a dose of 5×10¹³ (5.00E+13) in Cynomologus Macaques.

FIG. 26A shows eGFP expression (GFP pg/μg of protein) biodistribution in harvested tissue samples of major organs (right, middle, and left lobes of liver, left ventricle of a heart, gastroenemius, diaphragm, spleen) following administration of AAVhu68.CB7.CI.eGFP.WPRE.rBG, and AAVhu95M199.CB7.CI.eGFP.WPRE.rBG via IV at a dose of 5×10¹³ (5.00E+13) in Cynomologus Macaques.

FIG. 26B shows eGFP expression (GFP pg/μg of protein) biodistribution in harvested tissue samples of major organs (kidney, lung, spinal cord (cervical, thoracic, lumbar) and brain (cerebellum, cerebrum) following administration of AAVhu68.CB7.CI.eGFP.WPRE.rBG, and AAVhu95M199.CB7.CI.eGFP.WPRE.rBG via IV at a dose of 5×10¹³ (5.00E+13) in Cynomologus Macaques.

FIG. 27 shows percent GFP-positive area in samples of liver, gastrocnemius, heart, and brain (cerebrum) as quantified from Immunohistochemistry microscopy analysis, following administration of AAVhu68.CB7.CI.eGFP.WPRE.rBG, and AAVhu95M199.CB7.CI.eGFP.WPRE.rBG via IV at a dose of 5×10¹³ (5.00E+13) in Cynomologus Macaques.

FIG. 28A shows Vector DNA (GC/g DNA) biodistribution in harvested tissue samples of liver, gastroenemius, heart and brain following administration of and AAVhu95M199.CB7.CI.eGFP.WPRE.rBG via IV at a dose of 5×10¹³ (5.00E+13) in marmosets.

FIG. 28B shows RNA transcript (RNA transcript/100 ng) biodistribution in harvested tissue samples of liver, gastroenemius, heart and brain following administration of and AAVhu95M199.CB7.CI.eGFP.WPRE.rBG via IV at a dose of 5×10¹³ (5.00E+13) in marmosets.

FIG. 29A shows a representative Immunohistochemistry (IHC) image of DRG (cervical) tissue from GFP expression analysis following IV administration of AAVhu68.CB7.CI.eGFP.WPRE.rBG vector in NHPs at a dose of 5×10¹³ GC/kg.

FIG. 29B shows another representative IHC image of DRG (cervical) tissue from GFP expression analysis following IV administration of AAVhu68.CB7.CI.eGFP.WPRE.rBGvector in NHPs at a dose of 5×10¹³ GC/kg.

FIG. 29C shows a representative IHC image of DRG (cervical) tissue from GFP expression analysis following IV administration of AAVhu95.CB7.CI.eGFP.WPRE.rBGvector in NHPs at a dose of 5×10¹³ GC/kg.

FIG. 29D shows another representative IHC image of DRG (cervical) tissue from GFP expression analysis following IV administration of AAVhu95.CB7.CI.eGFP.WPRE.rBGvector in NHPs at a dose of 5×10¹³ GC/kg.

FIG. 29E shows a representative IHC image of DRG (thoracic) tissue from GFP expression analysis following IV administration of AAVhu68.CB7.CI.eGFP.WPRE.rBGvector in NHPs at a dose of 5×10¹³ GC/kg.

FIG. 29F shows another representative IHC image of DRG (thoracic) tissue from GFP expression analysis following IV administration of AAVhu68.CB7.CI.eGFP.WPRE.rBGvector in NHPs at a dose of 5×10¹³ GC/kg.

FIG. 29G shows a representative IHC image of DRG (thoracic) tissue from GFP expression analysis following IV administration of AAVhu95.CB7.CI.eGFP.WPRE.rBGvector in NHPs at a dose of 5×10¹² GC/kg.

FIG. 29H shows another representative IHC image of DRG (thoracic) tissue from GFP expression analysis following IV administration of AAVhu95.CB7.CI.eGFP.WPRE.rBGvector in NHPs at a dose of 5×10¹³ GC/kg.

FIG. 29I shows a representative IHC image of DRG (lumbar) tissue from GFP expression analysis following IV administration of AAVhu68.CB7.CI.eGFP.WPRE.rBG vector in NHPs at a dose of 5×10¹³ GC/kg.

FIG. 29J shows another representative IHC image of DRG (lumbar) tissue from GFP expression analysis following IV administration of AAVhu68.CB7.CI.eGFP.WPRE.rBGvector in NHPs at a dose of 5×10¹³ GC/kg.

FIG. 29K shows a representative IHC image of DRG (lumbar) tissue from GFP expression analysis following IV administration of AAVhu95.CB7.CI.eGFP.WPRE.rBGvector in NHPs at a dose of 5×10¹³ GC/kg.

FIG. 29L shows another representative IHC image of DRG (lumbar) tissue from GFP expression analysis following IV administration of AAVhu95.CB7.CI.eGFP.WPRE.rBGvector in NHPs at a dose of 5×10¹³ GC/kg.

### DETAILED DESCRIPTION OF THE INVENTION

Provided herein are nucleic acid sequences and amino acids of a novel isolated adeno-associated virus (AAV), which is termed herein AAVhu95, which is within clade F. AAVhu95 varies from another Clade F virus AAV9 (SEQ ID NO: 6) by one encoded amino acid at position 412 of vp1, SEQ ID NO: 2 (Table 1). AAVhu95 varies from another Clade F virus AAVhu68 (SEQ ID NO: 9) by three encoded amino acids at positions 67, 157 and 412 of vp1, SEQ ID NO: 2 (Table 1). Provided are novel AAVhu95 capsids and/or engineered AAV capsids having alanine (Ala or A) at position 67, alanine (Ala or A) at position 157, threonine (Thr or T) at position 412, and serine (Ser or S) at position 483 based on the numbering of SEQ ID NO: 2.

Provided herein are nucleic acid sequences and amino acids of a novel isolated adeno-associated virus (AAV), which is termed herein AAVhu96, which is within clade F. AAVhu96 varies from another Clade F virus AAV9 (SEQ ID NO: 6) by three encoded amino acids at position 157, 412 and 483 of vp1, SEQ ID NO: 2 (Table 1). AAVhu96 varies from another Clade F virus AAVhu68 (SEQ ID NO: 9) by four encoded amino acids at positions 67,157,412 and 483 of vp1, SEQ ID NO: 2 (Table 1). Provided are novel AAVhu96 capsids and/or engineered AAV capsids having alanine (Ala or A) at position 67, glutamic acid (Glu or E) at position 157 threonine (Thr or T) at position 412, and isoleucine (Ile or I) at position 483 based on the numbering of SEQ ID NO: 2.

TABLE 1

| | Amino Acid Position | | | |
|---|---|---|---|---|
| Capsid | 67 | 157 | 412 | 483 |
| AAV9 | A | A | Q | S |
| AAVhu95 | A | A | T | S |
| AAVhu96 | A | E | T | I |
| AAVhu68 | E | V | Q | S |
| AAVhu95 | A | A | T | S |
| AAVhu96 | A | E | T | I |

These AAV capsids, AAVhu95 and/or AAVhu96, described herein are useful for generating recombinant AAV (rAAV) vectors that are provide good yield and/or packaging efficiency, and providing rAAV vectors (i.e., rAAVhu95 and/or rAAVhu96) useful in transducing a number of different cell and tissue types. Such cells and tissue types may include, without limitation, lung, heart, muscle, liver, pancreas, kidney, nasal epithelial cells, cardiac muscle cells or cardiomyocytes, hepatocytes, pulmonary endothelial cells, myocytes, pulmonary epithelial cells, islet cells, acinar cells, renal cells, cells in the central nervous system or peripheral nervous system, including, brain, hippocampus, motor cortex, cerebellum, and motor neurons. Compositions containing these vectors are also provided. The methods described herein are directed to use of rAAV comprising AAVhu95 or AAVhu96 capsid to target tissues of interest for treatment of various diseases, disorders, syndromes, and/or conditions.

In some embodiments, provided is a recombinant AAVhu95 vector having an AAVhu95 capsid and a heterologous nucleic acid sequence comprising a transgene under the control of regulatory sequences, which direct expression thereof following delivery to a subject. In other embodiments, provided is a recombinant AAVhu96 vector having an AAVhu96 capsid and a nucleic acid encoding a transgene under the control of regulatory sequences, which direct expression thereof following delivery to a subject.

In another aspect, described herein are molecules which utilize the AAV capsid sequences described herein, including fragments thereof, for production of viral vectors useful in delivery of a heterologous gene or other nucleic acid sequences to a target cell. In one embodiment, the vectors useful in compositions and methods described herein contain, at a minimum, a sequence encoding an AAV capsid as described herein, e.g., an AAVhu95 capsid, or a fragment thereof. In another embodiment, the vectors useful in compositions and methods described herein contain, at a minimum, a sequence encoding an AAV capsid as described herein, e.g., an AAVhu96 capsid, or a fragment thereof. In yet another embodiment, useful vectors contain, at a minimum, sequences encoding a selected AAV serotype rep protein, or a fragment thereof. Optionally, such vectors may contain both AAV cap and rep proteins. In vectors in which both AAV rep and cap are provided, the AAV rep and AAV cap sequences can both be of one serotype origin, e.g., all an AAVhu95 or AAVhu96 origin. Alternatively, vectors may be used in which the rep sequences are from an AAV which differs from the wild type AAV providing the cap sequences. In one embodiment, the rep and cap sequences are expressed from separate sources (e.g., separate vectors, or a host cell and a vector). In one embodiment, the rep sequence is SEQ ID NO: 12. In another embodiment, these rep sequences are fused in frame to cap sequences of a different AAV serotype to form a chimeric AAV vector, such as AAV2/8 described in U.S. Pat. No. 7,282,199, which is incorporated by reference herein. Optionally, the vectors further contain a vector genome comprising an expression cassette comprising a selected transgene, wherein expression cassette is flanked by AAV 5′ ITR and AAV 3′ ITR. In certain embodiments, the AAV ITR sequences are from an AAV other than AAVhu95 or AAVhu96. In certain embodiments, the AAV ITR sequences are from AAV2. In another embodiment, the AAV is a self-complementary AAV (sc-AAV) (See, US 2012/0141422 which is incorporated herein by reference). Self-complementary vectors package an inverted repeat genome that can fold into dsDNA without the requirement for DNA synthesis or base-pairing between multiple vector genomes. Because scAAV have no need to convert the single-stranded DNA (ssDNA) genome into double-stranded DNA (dsDNA) prior to expression, they are more efficient vectors. However, the trade-off for this efficiency is the loss of half the coding capacity of the vector, scAAV are useful for small protein-coding genes (up to ˜55 kd) and any currently available RNA-based therapy. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

Pseudotyped vectors, wherein the capsid of one AAV is replaced with a heterologous capsid protein, are useful herein. For illustrative purposes, AAV vectors utilizing an AAVhu95 capsid or AAVhu96 capsid as described herein, with AAV2 ITRs are used in the examples described below. See, Mussolino et al, cited above. Unless otherwise specified, the AAV ITRs, and other selected AAV components described herein, may be individually selected from among any AAV serotype, including, without limitation, AAV1, AAV2, AAV3, AAV4, AAV5, AAV6, AAV7, AAV8, AAV9 or other known and unknown AAV serotypes. In one desirable embodiment, the ITRs of AAV serotype 2 (i.e., AAV2) are used. However, ITRs from other suitable serotypes may be selected. These ITRs or other AAV components may be readily isolated using techniques available to those of skill in the art from an AAV serotype. Such AAV may be isolated or obtained from academic, commercial, or public sources (e.g., the American Type Culture Collection, Manassas, VA). Alternatively, the AAV sequences may be obtained through synthetic or other suitable means by reference to published sequences such as are available in the literature or in databases such as, e.g., GenBank, PubMed, or the like.

A “recombinant AAV” or “rAAV” is a DNAse-resistant viral particle containing two elements, an AAV capsid and a vector genome containing at least non-AAV coding sequences (e.g., expression cassette within a vector genome) packaged within the AAV capsid. Unless otherwise specified, this term may be used interchangeably with the phrase “rAAV vector”. The rAAV is a “replication-defective virus” or “viral vector”, as it lacks any functional AAV rep gene or functional AAV cap gene and cannot generate progeny. In certain embodiments, the only AAV sequences are the AAV inverted terminal repeat sequences (ITRs), typically located at the extreme 5′ and 3′ ends of the vector genome (e.g., AAV 5′ ITR, expression cassette, AAV 3′ ITR) in order to allow the gene and regulatory sequences located between the ITRs to be packaged within the AAV capsid.

A rAAV is composed of an AAV capsid and a vector genome. An AAV capsid is an assembly of a heterogeneous population of vp1, a heterogeneous population of vp2, and a heterogeneous population of vp3 proteins. As used herein when used to refer to vp capsid proteins, the term “heterogeneous” or any grammatical variation thereof, refers to a population consisting of elements that are not the same, for example, having vp1, vp2 or vp3 monomers (proteins) with different modified amino acid sequences.

As used herein, the term “heterogeneous population” as used in connection with vp1, vp2 and vp3 proteins (alternatively termed isoforms), refers to differences in the amino acid sequence of the vp1, vp2 and vp3 proteins within a capsid. The AAV capsid contains subpopulations within the vp1 proteins, within the vp2 proteins and within the vp3 proteins which have modifications from the predicted amino acid residues. These subpopulations include, at a minimum, certain deamidated asparagine (N or Asn) residues. For example, certain subpopulations comprise at least one, two, three or four highly deamidated asparagines (N) positions in asparagine—glycine pairs and optionally further comprising other deamidated amino acids, wherein the deamidation results in an amino acid change and other optional modifications.

As used herein, a “subpopulation” of vp proteins refers to a group of vp proteins which has at least one defined characteristic in common and which consists of at least one group member to less than all members of the reference group, unless otherwise specified. For example, a “subpopulation” of vp1 proteins may be at least one (1) vp1 protein and less than all vp1 proteins in an assembled AAV capsid, unless otherwise specified. A “subpopulation” of vp3 proteins may be one (1) vp3 protein to less than all vp3 proteins in an assembled AAV capsid, unless otherwise specified. For example, vp1 proteins may be a subpopulation of vp proteins; vp2 proteins may be a separate subpopulation of vp proteins, and vp3 are yet a further subpopulation of vp proteins in an assembled AAV capsid. In another example, vp1, vp2 and vp3 (or VP1, VP2, and VP3) proteins may contain subpopulations having different modifications, e.g., at least one, two, three or four highly deamidated asparagines, e.g., at asparagine—glycine pairs. See PCT/US19/019804, filed Feb. 27, 2019, and PCT/US19/019861, filed Feb. 27, 2019, each of which is hereby incorporated by reference.

Unless otherwise specified, highly deamidated refers to at least 45% deamidated, at least 50% deamidated, at least 60% deamidated, at least 65% deamidated, at least 70%, at least 75%, at least 80%, at least 85%, at least 90%, at least 95%, at least 97%, at least 99%, or up to about 100% deamidated, 50% to 100% deamidated, 70% to 100% deamidated, 75% to 100% deamidated, or 70% to 90% deamidated, at a referenced amino acid position, as compared to the predicted amino acid sequence at the reference amino acid position. Such percentages may be determined using 2D-gel, mass spectrometry techniques, or other suitable techniques.

Without wishing to be bound by theory, the deamidation of at least highly deamidated residues in the vp proteins in the AAV capsid is believed to be primarily non-enzymatic in nature, being caused by functional groups within the capsid protein which deamidate selected asparagines, and to a lesser extent, glutamine residues. Efficient capsid assembly of the majority of deamidation vp1 proteins indicates that either these events occur following capsid assembly or that deamidation in individual monomers (vp1, vp2 or vp3) is well-tolerated structurally and largely does not affect assembly dynamics. Extensive deamidation in the VP1-unique (VP1-u) region (˜aa 1-137), generally considered to be located internally prior to cellular entry, suggests that VP deamidation may occur prior to capsid assembly.

Without wishing to be bound by theory, the deamidation of N may occur through its C-terminus residue's backbone nitrogen atom conducts a nucleophilic attack to the Asn's side chain amide group carbon atom. An intermediate ring-closed succinimide residue is believed to form. The succinimide residue then conducts fast hydrolysis to lead to the final product aspartic acid (Asp) or iso aspartic acid (IsoAsp). Therefore, in certain embodiments, the deamidation of asparagine (N or Asn) leads to an Asp or IsoAsp, which may interconvert through the succinimide intermediate. As provided herein, each deamidated N in the VP1, VP2 or VP3 may independently be aspartic acid (Asp), isoaspartic acid (isoAsp), aspartate, and/or an interconverting blend of Asp and isoAsp, or combinations thereof. Any suitable ratio of α- and isoaspartic acid may be present. For example, in certain embodiments, the ratio may be from 10:1 to 1:10 aspartic to isoaspartic, about 50:50 aspartic: isoaspartic, or about 1:3 aspartic: isoaspartic, or another selected ratio.

In certain embodiments, one or more glutamine (Q) may deamidates to glutamic acid (Glu), i.e., α-glutamic acid, γ-glutamic acid (Glu), or a blend of α- and γ-glutamic acid, which may interconvert through a common glutarimide intermediate. Any suitable ratio of α- and γ-glutamic acid may be present. For example, in certain embodiments, the ratio may be from 10:1 to 1:10 α to γ, about 50:50 α: γ, or about 1:3 α: γ, or another selected ratio.

Thus, an rAAV includes subpopulations within the rAAV capsid of vp1, vp2 and/or vp3 proteins with deamidated amino acids, including at a minimum, at least one subpopulation comprising at least one highly deamidated asparagine. In addition, other modifications may include isomerization, particularly at selected aspartic acid (D or Asp) residue positions. In still other embodiments, modifications may include an amidation at an Asp position.

In certain embodiments, an AAV capsid contains subpopulations of vp1, vp2 and vp3 having at least 1, at least 2, at least 3, at least 4, at least 5 to at least about 25 deamidated amino acid residue positions, of which at least 1 to 10%, at least 10 to 25%, at least 25 to 50%, at least 50 to 70%, at least 70 to 100%, at least 75 to 100%, at least 80-100% or at least 90-100% are deamidated as compared to the encoded amino acid sequence of the vp proteins. The majority of these may be N residues. However, Q residues may also be deamidated.

As used herein, “encoded amino acid sequence” refers to the amino acid which is predicted based on the translation of a known DNA codon of a referenced nucleic acid sequence being translated to an amino acid. The following table illustrates DNA codons and twenty common amino acids, showing both the single letter code (SLC) and three letter code (3LC).

| Amino Acid | SLC | 3 LC | DNA codons |
|---|---|---|---|
| Isoleucine | I | Ile | ATT, ATC, ATA |
| Leucine | L | Leu | CTT, CTC, CTA, CTG, TTA, TTG |
| Valine | V | Val | GTT, GTC, GTA, GTG |
| Phenylalanine | F | Phe | TTT, TTC |
| Methionine | M | Met | ATG |
| Cysteine | C | Cys | TGT, TGC |
| Alanine | A | Ala | GCT, GCC, GCA, GCG |
| Glycine | G | Gly | GGT, GGC, GGA, GGG |
| Proline | P | Pro | CCT, CCC, CCA, CCG |
| Threonine | T | Thr | ACT, ACC, ACA, ACG |
| Serine | S | Ser | TCT, TCC, TCA, TCG, AGT, AGC |
| Tyrosine | Y | Tyr | TAT, TAC |
| Tryptophan | W | Trp | TGG |
| Glutamine | Q | Gln | CAA, CAG |
| Asparagine | N | Asn | AAT, AAC |
| Histidine | H | His | CAT, CAC |
| Glutamic acid | E | Glu | GAA, GAG |
| Aspartic acid | D | Asp | GAT, GAC |
| Lysine | K | Lys | AAA, AAG |
| Arginine | R | Arg | CGT, CGC, CGA, CGG, AGA, AGG |
| Stop codons | Stop | | TAA, TAG, TGA |

In certain embodiments, a rAAV has an AAV capsid having vp1, vp2 and vp3 proteins having subpopulations comprising combinations of two, three, four, five or more deamidated residues at the positions set forth in the tables provided herein and incorporated herein by reference.

Deamidation in the rAAV may be determined using 2D gel electrophoresis, and/or mass spectrometry, and/or protein modelling techniques. Online chromatography may be performed with an Acclaim PepMap column and a Thermo UltiMate 3000 RSLC system (Thermo Fisher Scientific) coupled to a Q Exactive HF with a NanoFlex source (Thermo Fisher Scientific). MS data is acquired using a data-dependent top-20 method for the Q Exactive HF, dynamically choosing the most abundant not-yet-sequenced precursor ions from the survey scans (200-2000 m/z). Sequencing is performed via higher energy collisional dissociation fragmentation with a target value of 1e5 ions determined with predictive automatic gain control and an isolation of precursors was performed with a window of 4 m/z. Survey scans were acquired at a resolution of 120,000 at m/z 200. Resolution for HCD spectra may be set to 30,000 at m/z200 with a maximum ion injection time of 50 ms and a normalized collision energy of 30. The S-lens RF level may be set at 50, to give optimal transmission of the m/z region occupied by the peptides from the digest. Precursor ions may be excluded with single, unassigned, or six and higher charge states from fragmentation selection. BioPharma Finder 1.0 software (Thermo Fischer Scientific) may be used for analysis of the data acquired. For peptide mapping, searches are performed using a single-entry protein FASTA database with carbamidomethylation set as a fixed modification; and oxidation, deamidation, and phosphorylation set as variable modifications, a 10-ppm mass accuracy, a high protease specificity, and a confidence level of 0.8 for MS/MS spectra. Examples of suitable proteases may include, e.g., trypsin or chymotrypsin. Mass spectrometric identification of deamidated peptides is relatively straightforward, as deamidation adds to the mass of intact molecule +0.984 Da (the mass difference between —OH and —NH2 groups). The percent deamidation of a particular peptide is determined by mass area of the deamidated peptide divided by the sum of the area of the deamidated and native peptides. Considering the number of possible deamidation sites, isobaric species which are deamidated at different sites may co-migrate in a single peak. Consequently, fragment ions originating from peptides with multiple potential deamidation sites can be used to locate or differentiate multiple sites of deamidation. In these cases, the relative intensities within the observed isotope patterns can be used to specifically determine the relative abundance of the different deamidated peptide isomers. This method assumes that the fragmentation efficiency for all isomeric species is the same and independent on the site of deamidation. It will be understood by one of skill in the art that a number of variations on these illustrative methods can be used. For example, suitable mass spectrometers may include, e.g., a quadrupole time of flight mass spectrometer (QTOF), such as a Waters Xevo or Agilent 6530 or an orbitrap instrument, such as the Orbitrap Fusion or Orbitrap Velos (Thermo Fisher). Suitably liquid chromatography systems include, e.g., Acquity UPLC system from Waters or Agilent systems (1100 or 1200 series). Suitable data analysis software may include, e.g., MassLynx (Waters), Pinpoint and Pepfinder (Thermo Fischer Scientific), Mascot (Matrix Science), Peaks DB (Bioinformatics Solutions). Still other techniques may be described, e.g., in X. Jin et al, Hu Gene Therapy Methods, Vol. 28, No. 5, pp. 255-267, published online Jun. 16, 2017.

In addition to deamidations, other modifications may occur that do not result in conversion of one amino acid to a different amino acid residue. Such modifications may include acetylated residues, isomerizations, phosphorylations, or oxidations.

Modulation of Deamidation: In certain embodiments, the AAV is modified to change the glycine in an asparagine-glycine pair, to reduce deamidation. In other embodiments, the asparagine is altered to a different amino acid, e.g., a glutamine which deamidates at a slower rate; or to an amino acid which lacks amide groups (e.g., glutamine and asparagine contain amide groups); and/or to an amino acid which lacks amine groups (e.g., lysine, arginine and histidine contain amine groups). As used herein, amino acids lacking amide or amine side groups refer to, e.g., glycine, alanine, valine, leucine, isoleucine, serine, threonine, cystine, phenylalanine, tyrosine, or tryptophan, and/or proline. Modifications such as described may be in one, two, or three of the asparagine-glycine pairs found in the encoded AAV amino acid sequence. In certain embodiments, such modifications are not made in all four of the asparagine—glycine pairs. Thus, a method for reducing deamidation of AAV and/or engineered AAV variants having lower deamidation rates. Additionally, or alternatively, one or more other amide amino acids may be changed to a non-amide amino acid to reduce deamidation of the AAV. In certain embodiments, a mutant AAV capsid as described herein contains a mutation in an asparagine—glycine pair, such that the glycine is changed to an alanine or a serine. A mutant AAV capsid may contain one, two or three mutants where the reference AAV natively contains four NG pairs. In certain embodiments, an AAV capsid may contain one, two, three or four such mutants where the reference AAV natively contains five NG pairs. In certain embodiments, a mutant AAV capsid contains only a single mutation in an NG pair. In certain embodiments, a mutant AAV capsid contains mutations in two different NG pairs. In certain embodiments, a mutant AAV capsid contains mutation is two different NG pairs which are located in structurally separate location in the AAV capsid. In certain embodiments, the mutation is not in the VP1-unique region. In certain embodiments, one of the mutations is in the VP1-unique region. Optionally, a mutant AAV capsid contains no modifications in the NG pairs, but contains mutations to minimize or eliminate deamidation in one or more asparagines, or a glutamine, located outside of an NG pair.

In certain embodiments, a method of increasing the potency of a rAAV vector is provided which comprises engineering an AAV capsid which eliminating one or more of the NGs in the wild-type AAV capsid. In certain embodiments, the coding sequence for the “G” of the “NG” is engineered to encode another amino acid. In certain examples below, an “S” or an “A” is substituted. However, other suitable amino acid coding sequences may be selected.

These amino acid modifications may be made by conventional genetic engineering techniques. For example, a nucleic acid sequence containing modified AAV vp codons may be generated in which one to three of the codons encoding glycine in asparagine—glycine pairs are modified to encode an amino acid other than glycine. In certain embodiments, a nucleic acid sequence containing modified asparagine codons may be engineered at one to three of the asparagine—glycine pairs, such that the modified codon encodes an amino acid other than asparagine. Each modified codon may encode a different amino acid. Alternatively, one or more of the altered codons may encode the same amino acid. In certain embodiments, the modified AAVhu95 nucleic acid sequences is be used to generate a mutant rAAV having a capsid with lower deamidation than the native AAVhu95 capsid. In certain embodiments, the modified AAVhu96 nucleic acid sequences is be used to generate a mutant rAAV having a capsid with lower deamidation than the native AAVhu96 capsid. Such mutant rAAV may have reduced immunogenicity and/or increase stability on storage, particularly storage in suspension form.

Also provided herein are nucleic acid sequences encoding the AAV capsids having reduced deamidation. It is within the skill in the art to design nucleic acid sequences encoding this AAV capsid, including DNA (genomic or cDNA), or RNA (e.g., mRNA). Such nucleic acid sequences may be codon-optimized for expression in a selected system (i.e., cell type) and can be designed by various methods. This optimization may be performed using methods which are available on-line (e.g., GeneArt), published methods, or a company which provides codon optimizing services, e.g., DNA2.0 (Menlo Park, CA). One codon optimizing method is described, e.g., in International Patent Publication No. WO 2015/012924, which is incorporated by reference herein in its entirety. See also, e.g., US Patent Publication No. 2014/0032186 and US Patent Publication No. 2006/0136184. Suitably, the entire length of the open reading frame (ORF) for the product is modified. However, in some embodiments, only a fragment of the ORF may be altered. By using one of these methods, one can apply the frequencies to any given polypeptide sequence and produce a nucleic acid fragment of a codon-optimized coding region which encodes the polypeptide. A number of options are available for performing the actual changes to the codons or for synthesizing the codon-optimized coding regions designed as described herein. Such modifications or synthesis can be performed using standard and routine molecular biological manipulations well known to those of ordinary skill in the art. In one approach, a series of complementary oligonucleotide pairs of 80-90 nucleotides each in length and spanning the length of the desired sequence are synthesized by standard methods. These oligonucleotide pairs are synthesized such that upon annealing, they form double stranded fragments of 80-90 base pairs, containing cohesive ends, e.g., each oligonucleotide in the pair is synthesized to extend 3, 4, 5, 6, 7, 8, 9, 10, or more bases beyond the region that is complementary to the other oligonucleotide in the pair. The single-stranded ends of each pair of oligonucleotides are designed to anneal with the single-stranded end of another pair of oligonucleotides. The oligonucleotide pairs are allowed to anneal, and approximately five to six of these double-stranded fragments are then allowed to anneal together via the cohesive single stranded ends, and then they ligated together and cloned into a standard bacterial cloning vector, for example, a TOPO® vector available from Invitrogen Corporation, Carlsbad, Calif. The construct is then sequenced by standard methods. Several of these constructs consisting of 5 to 6 fragments of 80 to 90 base pair fragments ligated together, i.e., fragments of about 500 base pairs, are prepared, such that the entire desired sequence is represented in a series of plasmid constructs. The inserts of these plasmids are then cut with appropriate restriction enzymes and ligated together to form the final construct. The final construct is then cloned into a standard bacterial cloning vector, and sequenced. Additional methods would be immediately apparent to the skilled artisan. In addition, gene synthesis is readily available commercially.

In certain embodiments, AAV capsids are provided which have a heterogeneous population of AAV capsid isoforms (i.e., VP1, VP2, VP3) which contain multiple highly deamidated “NG” positions. In certain embodiments, the highly deamidated positions are in the locations identified below, with reference to the predicted full-length VP1 amino acid sequence. In other embodiments, the capsid gene is modified such that the referenced “NG” is ablated and a mutant “NG” is engineered into another position.

As used herein, a “stock” of rAAV refers to a population of rAAV. Despite heterogeneity in their capsid proteins due to deamidation, rAAV in a stock are expected to share an identical vector genome. A stock can include rAAV having capsids with, for example, heterogeneous deamidation patterns characteristic of the selected AAV capsid proteins and a selected production system. The stock may be produced from a single production system or pooled from multiple runs of the production system. A variety of production systems, including but not limited to those described herein, may be selected.

### AAV Capsid

Provided herein is a novel AAV capsid proteins having the vp1 sequence set forth in amino acid sequences of SEQ ID NOs: 2 and 4. The AAV capsid consists of three overlapping coding sequences, which vary in length due to alternative start codon usage. These variable proteins are referred to as VP1, VP2 and VP3, with VP1 being the longest and VP3 being the shortest. The AAV particle consists of all three capsid proteins at a ratio of 1:1:10 (VP1:VP2:VP3). VP3, which is comprised in VP1 and VP2 at the N-terminus, is the main structural component that builds the particle. The capsid protein can be referred to using several different numbering systems. For convenience, as used herein, the AAV sequences are referred to using VP1 numbering, which starts with aa 1 for the first residue of VP1. However, the capsid proteins described herein include VP1, VP2 and VP3 (used interchangeably herein with vp1, vp2 and vp3). The numbering of the variable proteins of the capsids are as follows:

Nucleotides (nt) (nucleic acid sequence encoding AAV capsid, 2208 nucleotides with a stop codon, i.e., 2211 nucleotides): AAVhu96: vp1-nt 1 to 2211 of SEQ ID NO: 10; vp2-nt 412 to 2211 of SEQ ID NO: 10 (or SEQ ID NO: 17); vp3-nt 607 to 2211 of SEQ ID NO: 10 (or SEQ ID NO: 18); AAVhu95 engineered: vp1-nt 1 to 2211 of SEQ ID NO: 1; vp2-nt 412 to 2211 of SEQ ID NO: 1 (or SEQ ID NO: 13); vp3-nt 607 to 2211 of SEQ ID NO: 1 (or SEQ ID NO: 14); AAVhu96: vp1-nt 1 to 2211 of SEQ ID NO: 11; vp2-nt 412 to 2211 of SEQ ID NO: 11 (or SEQ ID NO: 19); vp3-nt 607 to 2211 of SEQ ID NO: 11 (or SEQ ID NO: 20); AAVhu96 engineered: vp1-nt 1 to 2211 of SEQ ID NO: 3; vp2-nt 412 to 2211 of SEQ ID NO: 3 (or SEQ ID NO: 15); vp3-nt 607 to 2211 of SEQ ID NO: 3 (or SEQ ID NO: 16),
Amino acids (aa) AAVhu95 and AAVhu95 engineered: aa vp1-1 to 736 of SEQ ID NO: 2; vp2-aa 138 to 736 of SEQ ID NO: 2 (or SEQ ID NO: 21); vp3-aa 203 to 736 of SEQ ID NO: 2 (or SEQ ID NO: 22); AAVhu96 and AAVhu96 engineered: aa vp1-1 to 736 of SEQ ID NO: 4; vp2-aa 138 to 736 of SEQ ID NO: 4 (or SEQ ID NO: 23); vp3-aa 203 to 736 of SEQ ID NO: 4 (or SEQ ID NO: 24).

In certain embodiments, optionally, the nucleic acid sequence further comprises at least one or more additional stop codon (TAA, TAG, TGA).

Included herein are rAAV comprising at least one of the vp1, vp2 and the vp3 of AAVhu95 (SEQ ID NO: 2). Also provided herein are rAAV comprising AAV capsids encoded by at least one of the vp1, vp2 and the vp3 of AAVhu95 (SEQ ID NO: 10) or AAVhu95 engineered (SEQ ID NO: 1).

Included herein are rAAV comprising at least one of the vp1, vp2 and the vp3 of AAVhu96 (SEQ ID NO: 4). Also provided herein are rAAV comprising AAV capsids encoded by at least one of the vp1, vp2 and the vp3 of AAVhu96 (SEQ ID NO: 11) or AAVhu96 engineered (SEQ ID NO: 3).

In one embodiment, a composition is provided which includes a mixed population of recombinant adeno-associated virus (rAAV), each of said rAAV comprising: (a) an AAV capsid comprising about 60 capsid proteins made up of vp1 proteins, vp2 proteins and vp3 proteins, wherein the vp1, vp2 and vp3 proteins are: a heterogeneous population of vp1 proteins which are produced from a nucleic acid sequence encoding a selected AAV vp1 amino acid sequence, a heterogeneous population of vp2 proteins which are produced from a nucleic acid sequence encoding a selected AAV vp2 amino acid sequence, a heterogeneous population of vp3 proteins which produced from a nucleic acid sequence encoding a selected AAV vp3 amino acid sequence, wherein: the vp1, vp2 and vp3 proteins contain subpopulations with amino acid modifications comprising at least two highly deamidated asparagines (N) in asparagine—glycine pairs in the AAV capsid and optionally further comprising subpopulations comprising other deamidated amino acids, wherein the deamidation results in an amino acid change; and (b) a vector genome in the AAV capsid, the vector genome comprising a nucleic acid molecule comprising AAV inverted terminal repeat sequences and a non-AAV nucleic acid sequence (e.g., AAV 5′ ITR, expression cassette, AAV3′ ITR) encoding a product operably linked to sequences which direct expression of the product in a target cell.

In certain embodiments, the deamidated asparagines are deamidated to aspartic acid, isoaspartic acid, an interconverting aspartic acid/isoaspartic acid pair, or combinations thereof. In certain embodiments, the capsid further comprises deamidated glutamine(s) which are deamidated to (α)-glutamic acid, γ-glutamic acid, an interconverting (α)-glutamic acid/γ-glutamic acid pair, or combinations thereof.

In certain embodiments, a novel isolated AAVhu95 capsid is provided. A nucleic acid sequence encoding the AAVhu95 capsid is provided in SEQ ID NO: 10 and the encoded amino acid sequence is provided in SEQ ID NO: 2. Provided herein is an rAAV comprising at least one of the vp1, vp2 and the vp3 of AAVhu95 (SEQ ID NO: 2). Also provided herein are rAAV comprising an AAV capsid encoded by at least one of the vp1, vp2 and the vp3 of AAVhu95 (SEQ ID NO: 10). In yet another embodiment, a nucleic acid sequence encoding the AAVhu95 amino acid sequence is provided in SEQ ID NO: 1 and the encoded amino acid sequence is provided in SEQ ID NO: 2. Also provided herein are rAAV comprising an AAV capsid encoded by at least one of the vp1, vp2 and the vp3 of AAVhu95 engineered (SEQ ID NO: 1). In certain embodiments, the vp1, vp2 and/or vp3 is the full-length capsid protein of AAVhu96 (SEQ ID NO: 2). In other embodiments, the vp1, vp2 and/or vp3 has an N-terminal and/or a C-terminal truncation (e.g., truncation(s) of about 1 to about 10 amino acids).

In certain embodiments, a novel isolated AAVhu96 capsid is provided. A nucleic acid sequence encoding the AAVhu96 capsid is provided in SEQ ID NO: 11 and the encoded amino acid sequence is provided in SEQ ID NO: 4. Provided herein is an rAAV comprising at least one of the vp1, vp2 and the vp3 of AAVhu96 (SEQ ID NO: 4). Also provided herein are rAAV comprising an AAV capsid encoded by at least one of the vp1, vp2 and the vp3 of AAVhu96 (SEQ ID NO: 11). In yet another embodiment, a nucleic acid sequence encoding the AAVhu96 amino acid sequence is provided in SEQ ID NO: 3 and the encoded amino acid sequence is provided in SEQ ID NO: 4. Also provided herein are rAAV comprising an AAV capsid encoded by at least one of the vp1, vp2 and the vp3 of AAVhu96 engineered (SEQ ID NO: 3). In certain embodiments, the vp1, vp2 and/or vp3 is the full-length capsid protein of AAVhu96 (SEQ ID NO: 4). In other embodiments, the vp1, vp2 and/or vp3 has an N-terminal and/or a C-terminal truncation (e.g., truncation(s) of about 1 to about 10 amino acids).

In a further aspect, a recombinant adeno-associated virus (rAAV) is provided which comprises: (A) an AAVhu95 capsid comprising one or more of: (1) AAVhu95 capsid proteins comprising: a heterogeneous population of AAVhu95 vp1 proteins selected from: vp1 proteins produced by expression from a nucleic acid sequence which encodes the predicted amino acid sequence of 1 to 736 of SEQ ID NO: 2, vp1 proteins produced from SEQ ID NO: 10, or vp1 proteins produced from a nucleic acid sequence at least 99% identical to SEQ ID NO: 10 which encodes the predicted amino acid sequence of 1 to 736 of SEQ ID NO: 2, a heterogeneous population of AAVhu95 vp2 proteins selected from: vp2 proteins produced by expression from a nucleic acid sequence which encodes the predicted amino acid sequence of at least about amino acids 138 to 736 of SEQ ID NO: 2 (or SEQ ID NO: 21), vp2 proteins produced from a sequence comprising at least nucleotides 412 to 2211 of SEQ ID NO: 10 (or SEQ ID NO: 17), or vp2 proteins produced from a nucleic acid sequence at least 99% identical to at least nucleotides 412 to 2211 of SEQ ID NO: 10 (or SEQ ID NO: 17) which encodes the predicted amino acid sequence of at least about amino acids 138 to 736 of SEQ ID NO: 2 (or SEQ ID NO: 21), a heterogeneous population of AAVhu95 vp3 proteins selected from: vp3 proteins produced by expression from a nucleic acid sequence which encodes the predicted amino acid sequence of at least about amino acids 203 to 736 of SEQ ID NO: 2 (or SEQ ID NO: 22), vp3 proteins produced from a sequence comprising at least nucleotides 607 to 2211 of SEQ ID NO: 10 (or SEQ ID NO: 18), or vp3 proteins produced from a nucleic acid sequence at least 99% identical to at least nucleotides 607 to 2211 of SEQ ID NO: 10 (or SEQ ID NO: 18) which encodes the predicted amino acid sequence of at least about amino acids 203 to 736 of SEQ ID NO: 2 (or SEQ ID NO: 22); and/or (2) a heterogeneous population of vp1 proteins which are the product of a nucleic acid sequence encoding the amino acid sequence of SEQ ID NO: 2, a heterogeneous population of vp2 proteins which are the product of a nucleic acid sequence encoding the amino acid sequence of at least about amino acids 138 to 736 of SEQ ID NO: 2 (or SEQ ID NO: 21), and a heterogeneous population of vp3 proteins which are the product of a nucleic acid sequence encoding at least amino acids 203 to 736 of SEQ ID NO: 2 (or SEQ ID NO: 22), wherein: the vp1, vp2 and vp3 proteins contain subpopulations with amino acid modifications comprising at least two highly deamidated asparagines (N) in asparagine—glycine pairs in SEQ ID NO: 2 and optionally further comprising subpopulations comprising other deamidated amino acids, wherein the deamidation results in an amino acid change; and (B) a vector genome in the AAVhu95 capsid, the vector genome comprising a nucleic acid molecule comprising AAV inverted terminal repeat sequences and a non-AAV nucleic acid sequence (e.g., AAV 5′ ITR, expression cassette, AAV3′ ITR) encoding a product operably linked to sequences which direct expression of the product in a target cell.

In yet another aspect, a recombinant adeno-associated virus (rAAV) is provided which comprises: (A) an AAVhu95 capsid comprising one or more of: (1) AAVhu95 capsid proteins comprising: a heterogeneous population of AAVhu95 vp1 proteins selected from: vp1 proteins produced by expression from a nucleic acid sequence which encodes the predicted amino acid sequence of 1 to 736 of SEQ ID NO: 2, vp1 proteins produced from SEQ ID NO: 1, or vp1 proteins produced from a nucleic acid sequence at least 91% identical to SEQ ID NO: 1 which encodes the predicted amino acid sequence of 1 to 736 of SEQ ID NO: 2, a heterogeneous population of AAVhu95 vp2 proteins selected from: vp2 proteins produced by expression from a nucleic acid sequence which encodes the predicted amino acid sequence of at least about amino acids 138 to 736 of SEQ ID NO: 2 (or SEQ ID NO: 21), vp2 proteins produced from a sequence comprising at least nucleotides 412 to 2211 of SEQ ID NO: 1 (or SEQ ID NO: 13), or vp2 proteins produced from a nucleic acid sequence at least 91% identical to at least nucleotides 412 to 2211 of SEQ ID NO: 1 (or SEQ ID NO: 13) which encodes the predicted amino acid sequence of at least about amino acids 138 to 736 of SEQ ID NO: 2 (or SEQ ID NO: 21), a heterogeneous population of AAVhu95 vp3 proteins selected from: vp3 proteins produced by expression from a nucleic acid sequence which encodes the predicted amino acid sequence of at least about amino acids 203 to 736 of SEQ ID NO: 2 (or SEQ ID NO: 22), vp3 proteins produced from a sequence comprising at least nucleotides 607 to 2211 of SEQ ID NO: 1 (or SEQ ID NO: 14), or vp3 proteins produced from a nucleic acid sequence at least 91% identical to at least nucleotides 607 to 2211 of SEQ ID NO: 1 (or SEQ ID NO: 14) which encodes the predicted amino acid sequence of at least about amino acids 203 to 736 of SEQ ID NO: 2 (or SEQ ID NO: 22); and/or (2) a heterogeneous population of vp1 proteins which are the product of a nucleic acid sequence encoding the amino acid sequence of SEQ ID NO: 2, a heterogeneous population of vp2 proteins which are the product of a nucleic acid sequence encoding the amino acid sequence of at least about amino acids 138 to 736 of SEQ ID NO: 2 (or SEQ ID NO: 21), and a heterogeneous population of vp3 proteins which are the product of a nucleic acid sequence encoding at least amino acids 203 to 736 of SEQ ID NO: 2 (or SEQ ID NO: 22), wherein: the vp1, vp2 and vp3 proteins contain subpopulations with amino acid modifications comprising at least two highly deamidated asparagines (N) in asparagine—glycine pairs in SEQ ID NO: 2 and optionally further comprising subpopulations comprising other deamidated amino acids, wherein the deamidation results in an amino acid change; and (B) a vector genome in the AAVhu95 capsid, the vector genome comprising a nucleic acid molecule comprising AAV inverted terminal repeat sequences and a non-AAV nucleic acid sequence (e.g., AAV 5′ ITR, expression cassette, AAV3′ ITR) encoding a product operably linked to sequences which direct expression of the product in a target cell.

In a further aspect, a recombinant adeno-associated virus (rAAV) is provided which comprises: (A) an AAVhu96 capsid comprising one or more of: (1) AAVhu96 capsid proteins comprising: a heterogeneous population of AAVhu96 vp1 proteins selected from: vp1 proteins produced by expression from a nucleic acid sequence which encodes the predicted amino acid sequence of 1 to 736 of SEQ ID NO: 4, vp1 proteins produced from SEQ ID NO: 11, or vp1 proteins produced from a nucleic acid sequence at least 99% identical to SEQ ID NO: 11 which encodes the predicted amino acid sequence of 1 to 736 of SEQ ID NO: 4, a heterogeneous population of AAVhu96 vp2 proteins selected from: vp2 proteins produced by expression from a nucleic acid sequence which encodes the predicted amino acid sequence of at least about amino acids 138 to 736 of SEQ ID NO: 4 (or SEQ ID NO: 23), vp2 proteins produced from a sequence comprising at least nucleotides 412 to 2211 of SEQ ID NO: 11 (or SEQ ID NO: 19), or vp2 proteins produced from a nucleic acid sequence at least 99% identical to at least nucleotides 412 to 2211 of SEQ ID NO: 11 (or SEQ ID NO: 19) which encodes the predicted amino acid sequence of at least about amino acids 138 to 736 of SEQ ID NO: 4 (or SEQ ID NO: 23), a heterogeneous population of AAVhu96 vp3 proteins selected from: vp3 proteins produced by expression from a nucleic acid sequence which encodes the predicted amino acid sequence of at least about amino acids 203 to 736 of SEQ ID NO: 4 (or SEQ ID NO: 24), vp3 proteins produced from a sequence comprising at least nucleotides 607 to 2211 of SEQ ID NO: 11 (or SEQ ID NO: 20), or vp3 proteins produced from a nucleic acid sequence at least 99% identical to at least nucleotides 607 to 2211 of SEQ ID NO: 11 (or SEQ ID NO: 20) which encodes the predicted amino acid sequence of at least about amino acids 203 to 736 of SEQ ID NO: 4 (or SEQ ID NO: 24); and/or (2) a heterogeneous population of vp1 proteins which are the product of a nucleic acid sequence encoding the amino acid sequence of SEQ ID NO: 4, a heterogeneous population of vp2 proteins which are the product of a nucleic acid sequence encoding the amino acid sequence of at least about amino acids 138 to 736 of SEQ ID NO: 4 (or SEQ ID NO: 23), and a heterogeneous population of vp3 proteins which are the product of a nucleic acid sequence encoding at least amino acids 203 to 736 of SEQ ID NO: 4 (or SEQ ID NO: 24), wherein: the vp1, vp2 and vp3 proteins contain subpopulations with amino acid modifications comprising at least two highly deamidated asparagines (N) in asparagine—glycine pairs in SEQ ID NO: 4 and optionally further comprising subpopulations comprising other deamidated amino acids, wherein the deamidation results in an amino acid change; and (B) a vector genome in the AAVhu95 capsid, the vector genome comprising a nucleic acid molecule comprising AAV inverted terminal repeat sequences and a non-AAV nucleic acid sequence (e.g., AAV 5′ ITR, expression cassette, AAV3′ ITR) encoding a product operably linked to sequences which direct expression of the product in a target cell.

In yet another aspect, a recombinant adeno-associated virus (rAAV) is provided which comprises: (A) an AAVhu96 capsid comprising one or more of: (1) AAVhu96 capsid proteins comprising: a heterogeneous population of AAVhu96 vp1 proteins selected from: vp1 proteins produced by expression from a nucleic acid sequence which encodes the predicted amino acid sequence of 1 to 736 of SEQ ID NO: 4, vp1 proteins produced from SEQ ID NO: 3, or vp1 proteins produced from a nucleic acid sequence at least 91% identical to SEQ ID NO: 3 which encodes the predicted amino acid sequence of 1 to 736 of SEQ ID NO: 4, a heterogeneous population of AAVhu96 vp2 proteins selected from: vp2 proteins produced by expression from a nucleic acid sequence which encodes the predicted amino acid sequence of at least about amino acids 138 to 736 of SEQ ID NO: 4 (or SEQ ID NO: 23), vp2 proteins produced from a sequence comprising at least nucleotides 412 to 2211 of SEQ ID NO: 3 (or SEQ ID NO: 15), or vp2 proteins produced from a nucleic acid sequence at least 91% identical to at least nucleotides 412 to 2211 of SEQ ID NO: 3 (or SEQ ID NO: 15) which encodes the predicted amino acid sequence of at least about amino acids 138 to 736 of SEQ ID NO: 4 (or SEQ ID NO: 23), a heterogeneous population of AAVhu96 vp3 proteins selected from: vp3 proteins produced by expression from a nucleic acid sequence which encodes the predicted amino acid sequence of at least about amino acids 203 to 736 of SEQ ID NO: 4 (or SEQ ID NO: 24), vp3 proteins produced from a sequence comprising at least nucleotides 607 to 2211 of SEQ ID NO: 3 (or SEQ ID NO: 16), or vp3 proteins produced from a nucleic acid sequence at least 91% identical to at least nucleotides 607 to 2211 of SEQ ID NO: 3 (or SEQ ID NO: 16) which encodes the predicted amino acid sequence of at least about amino acids 203 to 736 of SEQ ID NO: 4 (or SEQ ID NO: 24); and/or (2) a heterogeneous population of vp1 proteins which are the product of a nucleic acid sequence encoding the amino acid sequence of SEQ ID NO: 4, a heterogeneous population of vp2 proteins which are the product of a nucleic acid sequence encoding the amino acid sequence of at least about amino acids 138 to 736 of SEQ ID NO: 4 (or SEQ ID NO: 23), and a heterogeneous population of vp3 proteins which are the product of a nucleic acid sequence encoding at least amino acids 203 to 736 of SEQ ID NO: 4 (or SEQ ID NO: 24), wherein: the vp1, vp2 and vp3 proteins contain subpopulations with amino acid modifications comprising at least two highly deamidated asparagines (N) in asparagine—glycine pairs in SEQ ID NO: 4 and optionally further comprising subpopulations comprising other deamidated amino acids, wherein the deamidation results in an amino acid change; and (B) a vector genome in the AAVhu96 capsid, the vector genome comprising a nucleic acid molecule comprising AAV inverted terminal repeat sequences and a non-AAV nucleic acid sequence (e.g., AAV 5′ ITR, expression cassette, AAV3′ ITR) encoding a product operably linked to sequences which direct expression of the product in a target cell.

In certain embodiments, an AAVhu95 capsid comprises: a heterogeneous population of vp1 proteins which are the product of a nucleic acid sequence encoding the amino acid sequence of SEQ ID NO: 2, a heterogeneous population of vp2 proteins which are the product of a nucleic acid sequence encoding the amino acid sequence of at least about amino acids 138 to 736 of SEQ ID NO: 2 (or SEQ ID NO: 21), and a heterogeneous population of vp3 proteins which are the product of a nucleic acid sequence encoding at least amino acids 203 to 736 of SEQ ID NO: 2 (or SEQ ID NO: 22).

In certain embodiments, an AAVhu96 capsid comprises: a heterogeneous population of vp1 proteins which are the product of a nucleic acid sequence encoding the amino acid sequence of SEQ ID NO: 4, a heterogeneous population of vp2 proteins which are the product of a nucleic acid sequence encoding the amino acid sequence of at least about amino acids 138 to 736 of SEQ ID NO: 4 (or SEQ ID NO: 23), and a heterogeneous population of vp3 proteins which are the product of a nucleic acid sequence encoding at least amino acids 203 to 736 of SEQ ID NO: 4 (or SEQ ID NO: 24).

The invention also encompasses nucleic acid sequences encoding the AAVhu95 capsid sequence (SEQ ID NO: 2) or a mutant AAVhu95, in which one or more residues has been altered in order to decrease deamidation, or other modifications which are identified herein. Such nucleic acid sequences can be used in production of mutant AAVhu95 capsids.

Furthermore, the invention also encompasses nucleic acid sequences encoding the AAVhu96 capsid sequence (SEQ ID NO: 4) or a mutant AAVhu96, in which one or more residues has been altered in order to decrease deamidation, or other modifications which are identified herein. Such nucleic acid sequences can be used in production of mutant AAVhu96 capsids.

In certain embodiments, provided herein is a recombinant nucleic acid molecule having the sequence of SEQ ID NO: 10 or a sequence at least at least 99%, or 100% identical to SEQ ID NO: 10 which encodes the vp1 amino acid sequence of SEQ ID NO: 2 with a modification (e.g., deamidated amino acid) as described herein. In certain embodiments, provided herein is a recombinant nucleic acid molecule having the sequence of SEQ ID NO: 1 or a sequence at least 91%, or 100% identical to SEQ ID NO: 1 which encodes the vp1 amino acid sequence of SEQ ID NO: 2 with a modification (e.g., deamidated amino acid) as described herein. In certain embodiments, the vp1 amino acid sequence is reproduced in SEQ ID NO: 2. In certain embodiments, the recombinant nucleic acid molecule is a plasmid. In certain embodiments, a plasmid having a nucleic acid sequence described herein is provided. Such plasmids include a nucleic acid sequence that encodes at least one of the vp1, vp2, and vp3 of AAVhu95 (SEQ ID NOs: 10, 17, 18), or a sequence sharing at least 99% identity with a vp1, vp2, and/or vp3 sequence of SEQ ID NOs: 10, 17, and/or 18. In certain embodiments, the plasmid includes a nucleic acid sequence that encodes at least one of the vp1, vp2, and vp3 of AAVhu95 (SEQ ID NOs: 1, 13, 14), or a sequence sharing at least 91% identity with a vp1, vp2, and/or vp3 sequence of SEQ ID NOs: 1, 13, and/or 14. In further embodiments, the plasmids include a non-AAV sequence. In certain embodiments, the plasmid comprises a WPRE and/or bGH-polyA signal. Cultured production host cells containing the plasmids described herein are also provided.

In other embodiments, provided herein is a nucleic acid molecule having the sequence of SEQ ID NO: 11 or a sequence at least at least 99%, or 100% identical to SEQ ID NO: 11 which encodes the vp1 amino acid sequence of SEQ ID NO: 4 with a modification (e.g., deamidated amino acid) as described herein. In certain embodiments, provided herein is a nucleic acid molecule having the sequence of SEQ ID NO: 3 or a sequence at least 91%, or 100% identical to SEQ ID NO: 3 which encodes the vp1 amino acid sequence of SEQ ID NO: 4 with a modification (e.g., deamidated amino acid) as described herein. In certain embodiments, the vp1 amino acid sequence is reproduced in SEQ ID NO: 4. In certain embodiments, a plasmid having a nucleic acid sequence described herein is provided. Such plasmids include a nucleic acid sequence that encodes at least one of the vp1, vp2, and vp3 of AAVhu96 (SEQ ID NOs: 11, 19, 20), or a sequence sharing at least 99% identity with a vp1, vp2, and/or vp3 sequence of SEQ ID NOs: 11, 19, and/or 20. In certain embodiments, the plasmid includes a nucleic acid sequence that encodes at least one of the vp1, vp2, and vp3 of AAVhu96 (SEQ ID NOs: 3, 15, 16), or a sequence sharing at least 91% identity with a vp1, vp2, and/or vp3 sequence of SEQ ID NOs: 3, 15, and/or 16. In further embodiments, the plasmids include a non-AAV sequence. In certain embodiments, the plasmid comprises a WPRE and/or bGH-polyA signal. Cultured production host cells containing the plasmids described herein are also provided.

As used herein, the “conservative amino acid replacement” or “conservative amino acid substitutions” refers to a change, replacement or substitution of an amino acid to a different amino acid with similar biochemical properties (e.g., charge, hydrophobicity and size), which is known by practitioners of the art. Also see, e.g., FRENCH et al. What is a conservative substitution? Journal of Molecular Evolution, March 1983, Volume 19, Issue 2, pp 171-175 and YAMPOLSKY et al. The Exchangeability of Amino Acids in Proteins, Genetics. 2005 August; 170(4): 1459-1472, each of which is incorporated herein by reference in its entirety.

The term “substantial homology” or “substantial similarity,” when referring to a nucleic acid, or fragment thereof, indicates that, when optimally aligned with appropriate nucleotide insertions or deletions with another nucleic acid (or its complementary strand), there is nucleotide sequence identity in at least about 95 to 99% of the aligned sequences. Preferably, the homology is over full-length sequence, or an open reading frame thereof, or another suitable fragment which is at least 15 nucleotides in length. Examples of suitable fragments are described herein.

The term “percent (%) identity”, “sequence identity”, “percent sequence identity”, or “percent identical” in the context of nucleic acid sequences refers to the residues in the two sequences which are the same when aligned for correspondence. The length of sequence identity comparison may be over the full-length of the genome, the full-length of a gene coding sequence, or a fragment of at least about 500 to 5000 nucleotides, is desired. However, identity among smaller fragments, e.g., of at least about nine nucleotides, usually at least about 20 to 24 nucleotides, at least about 28 to 32 nucleotides, at least about 36 or more nucleotides, may also be desired.

By the term “highly conserved” is meant at least 80% identity, preferably at least 90% identity, and more preferably, over 97% identity. Identity is readily determined by one of skill in the art by resort to algorithms and computer programs known by those of skill in the art.

Unless otherwise specified by an upper range, it will be understood that a percentage of identity is a minimum level of identity and encompasses all higher levels of identity up to 100% identity to the reference sequence. Unless otherwise specified, it will be understood that a percentage of identity is a minimum level of identity and encompasses all higher levels of identity up to 100% identity to the reference sequence. For example, “⁹⁵% identity” and “at least 95% identity” may be used interchangeably and include 95%, 96%, 97%, 98%, 99%, and up to 100% identity to the referenced sequence, and all fractions therebetween.

Percent identity may be readily determined for amino acid sequences over the full-length of a protein, polypeptide, about 32 amino acids, about 330 amino acids, or a peptide fragment thereof or the corresponding nucleic acid sequence coding sequences. A suitable amino acid fragment may be at least about 8 amino acids in length, and may be up to about 700 amino acids. Generally, when referring to “identity”, “homology”, or “similarity” between two different sequences, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence.

Identity may be determined by preparing an alignment of the sequences and through the use of a variety of algorithms and/or computer programs known in the art or commercially available [e.g., BLAST, ExPASy; ClustalO; FASTA; using, e.g., Needleman-Wunsch algorithm, Smith-Waterman algorithm]. Alignments are performed using any of a variety of publicly or commercially available Multiple Sequence Alignment Programs. Sequence alignment programs are available for amino acid sequences, e.g., the “Clustal Omega”, “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

Multiple sequence alignment programs are also available for nucleic acid sequences. Examples of such programs include, “Clustal Omega”, “Clustal W”, “CAP Sequence Assembly”, “BLAST”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™, a program in GCG Version 6.1. Fasta™ provides alignments and percent sequence identity of the regions of the best overlap between the query and search sequences. For instance, percent sequence identity between nucleic acid sequences can be determined using Fasta™ with its default parameters (a word size of 6 and the NOPAM factor for the scoring matrix) as provided in GCG Version 6.1, herein incorporated by reference.

### Expression Cassette and Vectors

As used herein, an “expression cassette” refers to a nucleic acid molecule which comprises a biologically useful nucleic acid sequence (e.g., a gene cDNA encoding a protein, enzyme or other useful gene product, mRNA, etc.) and regulatory sequences operably linked thereto which direct or modulate transcription, translation, and/or expression of the nucleic acid sequence and its gene product. As used herein, “operably linked” sequences include both regulatory sequences that are contiguous or non-contiguous with the nucleic acid sequence and regulatory sequences that act in trans or cis nucleic acid sequence. Such regulatory sequences typically include, e.g., one or more of a promoter, an enhancer, an intron, a Kozak sequence, a polyadenylation sequence, and a TATA signal. The expression cassette may contain regulatory sequences upstream (5′ to) of the gene sequence, e.g., one or more of a promoter, an enhancer, an intron, etc., and one or more of an enhancer, or regulatory sequences downstream (3′ to) a gene sequence, e.g., 3′ untranslated region (3′ UTR) comprising a polyadenylation site, among other elements. In certain embodiments, the regulatory sequences are operably linked to the nucleic acid sequence of a gene product, wherein the regulatory sequences are separated from nucleic acid sequence of a gene product by an intervening nucleic acid sequences, i.e., 5′-untranslated regions (5′UTR). In certain embodiments, the expression cassette comprises nucleic acid sequence of one or more of gene products. In some embodiments, the expression cassette can be a monocistronic or a bicistronic expression cassette. In other embodiments, the term “transgene” refers to one or more DNA sequences from an exogenous source which are inserted into a target cell.

Typically, such an expression cassette can be used for generating a viral vector and contains the coding sequence for the gene product described herein flanked by packaging signals of the viral genome and other expression control sequences such as those described herein. In certain embodiments, a vector genome may contain two or more expression cassettes.

As used herein, a “vector genome” refers to the nucleic acid sequence packaged inside a parvovirus (e.g., rAAV) capsid which forms a viral particle. Such a nucleic acid sequence contains AAV inverted terminal repeat sequences (ITRs). In the examples herein, a vector genome contains, at a minimum, from 5′ to 3′, an AAV 5′ ITR, expression cassette comprising coding sequence(s) (i.e., transgene(s)), and an AAV 3′ ITR. ITRs from AAV2, a different source AAV than the capsid, or other than full-length ITRs may be selected. In certain embodiments, the ITRs are from the same AAV source as the AAV which provides the rep function during production or a transcomplementing AAV. Further, other ITRs, e.g., self-complementary (scAAV) ITRs, may be used. Both single-stranded AAV and self-complementary (sc) AAV are encompassed with the rAAV. The transgene is a nucleic acid coding sequence, heterologous to the vector sequences, which encodes a polypeptide, protein, functional RNA molecule (e.g., miRNA, miRNA inhibitor) or other gene product, of interest. The nucleic acid coding sequence is operatively linked to regulatory components in a manner which permits transgene transcription, translation, and/or expression in a cell of a target tissue. Suitable components of a vector genome are discussed in more detail herein.

Vector genomic sequences which are packaged into an AAVhu95 capsid or AAVhu96 capsid and delivered to a target cell are typically composed of, at a minimum, a transgene and its regulatory sequences, and AAV inverted terminal repeats (ITRs) (e.g., AAV 5′ ITR, expression cassette, and AAV 3′ ITR). Both single-stranded AAV and self-complementary (sc) AAV are encompassed with the rAAV. The transgene is a nucleic acid coding sequence, heterologous to the vector sequences, which encodes a polypeptide, protein, functional RNA molecule (e.g., miRNA, miRNA inhibitor) or other gene product, of interest. The nucleic acid coding sequence is operatively linked to regulatory components in a manner which permits transgene transcription, translation, and/or expression in a cell of a target tissue.

The AAV sequences of the vector typically comprise the cis-acting 5′ and 3′ inverted terminal repeat sequences (See, e.g., B. J. Carter, in “Handbook of Parvoviruses”, ed., P. Tijsser, CRC Press, pp. 155 168 (1990)). The ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al, “Molecular Cloning. A Laboratory Manual”, 2d ed., Cold Spring Harbor Laboratory, New York (1989); and K. Fisher et al., J. Virol., 70:520 532 (1996)). An example of such a molecule employed in the present invention is a “cis-acting” plasmid containing the transgene, in which the selected transgene sequence and associated regulatory elements are flanked by the 5′ and 3′ AAV ITR sequences. In one embodiment, the ITRs are from an AAV different than that supplying a capsid. In one embodiment, the ITR sequences from AAV2. However, ITRs from other AAV sources may be selected. A shortened version of the 5′ ITR, termed ΔITR, has been described in which the D-sequence and terminal resolution site (trs) are deleted. In certain embodiments, the vector genome includes a shortened AAV2 ITR of 130 base pairs, wherein the external A elements is deleted. Without wishing to be bound by theory, it is believed that the shortened ITR reverts back to the wild-type length of 145 base pairs during vector DNA amplification using the internal (A′) element as a template. In other embodiments, full-length AAV 5′ and 3′ ITRs are used. Where the source of the ITRs is from AAV2 and the AAV capsid is from another AAV source, the resulting vector may be termed pseudotyped. However, other configurations of these elements may be suitable.

In addition to the major elements identified above for the recombinant AAV vector, the vector also includes conventional control elements (i.e., regulatory sequences, expression control sequences) necessary which are operably linked to the transgene in a manner which permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest. As described herein, regulatory sequences comprise but not limited to: promoter; enhancer; transcription factor; transcription terminator; efficient RNA processing signals such as splicing and polyadenylation signals (polyA); sequences that stabilize cytoplasmic mRNA, for example Woodchuck Hepatitis Virus (WHP) Posttranscriptional Regulatory Element (WPRE); sequences that enhance translation efficiency (i.e., Kozak consensus sequence).

The regulatory control elements (regulatory elements) typically contain a promoter sequence as part of the expression control sequences, e.g., located between the selected 5′ ITR sequence and the coding sequence. Constitutive promoters, regulatable promoters [see, e.g., WO 2011/126808 and WO 2013/04943], tissue specific promoters, or a promoter responsive to physiologic cues may be used may be utilized in the vectors described herein.

Examples of constitutive promoters suitable for controlling expression of the therapeutic products include, but are not limited to chicken β-actin (CB) promoter, CB7 promoter, human cytomegalovirus (CMV) promoter, ubiquitin C promoter (UbC), the early and late promoters of simian virus 40 (SV40), U6 promoter, metallothionein promoters, EF1α promoter, ubiquitin promoter, hypoxanthine phosphoribosyl transferase (HPRT) promoter, dihydrofolate reductase (DHFR) promoter (Scharfmann et al., Proc. Natl. Acad. Sci. USA 88:4626-4630 (1991), adenosine deaminase promoter, phosphoglycerol kinase (PGK) promoter, pyruvate kinase promoter phosphoglycerol mutase promoter, the β-actin promoter (Lai et al., Proc. Natl. Acad. Sci. USA 86: 10006-10010 (1989)), the long terminal repeats (LTR) of Moloney Leukemia Virus and other retroviruses, the thymidine kinase promoter of Herpes Simplex Virus and other constitutive promoters known to those of skill in the art. Examples of tissue- or cell-specific promoters suitable for use in the present invention include, but are not limited to, endothelin-I (ET-I) and Flt-I, which are specific for endothelial cells, FoxJ1 (that targets ciliated cells). Other examples of tissue specific promoters suitable for use in the present invention include, but are not limited to, promoters useful in the peripheral or central nervous system (e.g., neurons or subsets thereof). In certain embodiments, the promoter is a neuron-specific promoter. Examples of such promoters may include, e.g., an elongation factor 1 alpha (EF1 alpha) promoter (see, e.g., Kim D W et al, Use of the human elongation factor 1 alpha promoter as a versatile and efficient expression system. Gene. 1990 Jul. 16; 91(2):217-23), a Synapsin 1 promoter (see, e.g., Kügler S et al, Human synapsin 1 gene promoter confers highly neuron-specific long-term transgene expression from an adenoviral vector in the adult rat brain depending on the transduced area. Gene Ther. 2003 February; 10(4):337-47), a shorted synapsin promoter, a neuron-specific enolase (NSE) promoter (see, e.g., Kim J et al, Involvement of cholesterol-rich lipid rafts in interleukin-6-induced neuroendocrine differentiation of LNCaP prostate cancer cells. Endocrinology. 2004 February; 145(2):613-9. Epub 2003 Oct. 16), or a CB6 promoter (see, e.g., Large-Scale Production of Adeno-Associated Viral Vector Serotype-9 Carrying the Human Survival Motor Neuron Gene, Mol Biotechnol., 2016 January; 58(1):30-6. doi: 10.1007/s12033-015-9899-5). In other embodiments, selection of cardiac-specific promoters may be desired. See, e.g., R. M. Deviatiirov, et al, “Human library of cardiac promoters and enhancers”, bioRxiv, pp. 1-27, bioRxiv preprint doi: https://doi.org/10.1101/2020.06.14.150904; posted Jun. 15, 2020. Preferably, such promoters are of human origin.

Inducible and regulatable promoters suitable for controlling expression of the therapeutic product include promoters responsive to exogenous agents (e.g., pharmacological agents) or to physiological cues. These response elements include, but are not limited to a hypoxia response element (HRE) that binds HIF-Iα and β, a metal-ion response element such as described by Mayo et al. (1982, Cell 29:99-108); Brinster et al. (1982, Nature 296:39-42) and Searle et al. (1985, Mol. Cell. Biol. 5:1480-1489); or a heat shock response element such as described by Nouer et al. (in: Heat Shock Response, ed. Nouer, L., CRC, Boca Raton, Fla., ppI67-220, 1991). In one embodiment, expression of the gene product is controlled by a regulatable promoter that provides tight control over the transcription of the sequence encoding the gene product, e.g., a pharmacological agent, or transcription factors activated by a pharmacological agent or in alternative embodiments, physiological cues. Promoter systems that are non-leaky and that can be tightly controlled are preferred. Examples of regulatable promoters which are ligand-dependent transcription factor complexes that may be used in the invention include, without limitation, members of the nuclear receptor superfamily activated by their respective ligands (e.g., glucocorticoid, estrogen, progestin, retinoid, ecdysone, and analogs and mimetics thereof) and rTTA activated by tetracycline. In one aspect of the invention, the gene switch is an EcR-based gene switch. Examples of such systems include, without limitation, the systems described in U.S. Pat. Nos. 6,258,603, 7,045,315, U.S. Published Patent Application Nos. 2006/0014711, 2007/0161086, and International Published Application No. WO 01/70816. Examples of chimeric ecdysone receptor systems are described in U.S. Pat. No. 7,091,038, U.S. Published Patent Application Nos. 2002/0110861, 2004/0033600, 2004/0096942, 2005/0266457, and 2006/0100416, and International Published Application Nos. WO 01/70816, WO 02/066612, WO 02/066613, WO 02/066614, WO 02/066615, WO 02/29075, and WO 2005/108617, each of which is incorporated by reference in its entirety. An example of a non-steroidal ecdysone agonist-regulated system is the RheoSwitch® Mammalian Inducible Expression System (New England Biolabs, Ipswich, MA).

Still other promoter systems may include response elements including but not limited to a tetracycline (tet) response element (such as described by Gossen & Bujard (1992, Proc. Natl. Acad. Sci. USA 89:5547-551); or a hormone response element such as described by Lee et al. (1981, Nature 294:228-232); Hynes et al. (1981, Proc. Natl. Acad. Sci. USA 78:2038-2042); Klock et al. (1987, Nature 329:734-736); and Israel & Kaufman (1989, Nucl. Acids Res. 17:2589-2604) and other inducible promoters known in the art. Using such promoters, expression of the soluble hACE2 construct can be controlled, for example, by the Tet-on/off system (Gossen et al., 1995, Science 268:1766-9; Gossen et al., 1992, Proc. Natl. Acad. Sci. USA., 89(12):5547-51); the TetR-KRAB system (Urrutia R., 2003, Genome Biol., 4(10):231; Deuschle U et al., 1995, Mol Cell Biol. (4):1907-14); the mifepristone (RU486) regulatable system (Geneswitch; Wang Y et al., 1994, Proc. Natl. Acad. Sci. USA., 91(17):8180-4; Schillinger et al., 2005, Proc. Natl. Acad. Sci. USA. 102(39):13789-94); and the humanized tamoxifen-dep regulatable system (Roscilli et al., 2002, Mol. Ther. 6(5):653-63).

In another aspect, the gene switch is based on heterodimerization of FK506 binding protein (FKBP) with FKBP rapamycin associated protein (FRAP) and is regulated through rapamycin or its non-immunosuppressive analogs. Examples of such systems, include, without limitation, the ARGENT™ Transcriptional Technology (ARIAD Pharmaceuticals, Cambridge, Mass.) and the systems described in U.S. Pat. Nos. 6,015,709, 6,117,680, 6,479,653, 6,187,757, and 6,649,595, U.S. Publication No. 2002/0173474, U.S. Publication No. 200910100535, U.S. Pat. Nos. 5,834,266, 7,109,317, 7,485,441, 5,830,462, 5,869,337, 5,871,753, 6,011,018, 6,043,082, 6,046,047, 6,063,625, 6,140,120, 6,165,787, 6,972,193, 6,326,166, 7,008,780, 6,133,456, 6,150,527, 6,506,379, 6,258,823, 6,693,189, 6,127,521, 6,150,137, 6,464,974, 6,509,152, 6,015,709, 6,117,680, 6,479,653, 6,187,757, 6,649,595, 6,984,635, 7,067,526, 7,196,192, 6,476,200, 6,492,106, WO 94/18347, WO 96/20951, WO 96/06097, WO 97/31898, WO 96/41865, WO 98/02441, WO 95/33052, WO 99110508, WO 99110510, WO 99/36553, WO 99/41258,WO 01114387, ARGENT™ Regulated Transcription Retrovirus Kit, Version 2.0 (9109102), and ARGENT™ Regulated Transcription Plasmid Kit, Version 2.0 (9109/02), each of which is incorporated herein by reference in its entirety. The Ariad system is designed to be induced by rapamycin and analogs thereof referred to as “rapalogs”. Examples of suitable rapamycins are provided in the documents listed above in connection with the description of the ARGENT™ system. In one embodiment, the molecule is rapamycin [e.g., marketed as Rapamune™ by Pfizer]. In another embodiment, a rapalog known as AP21967 [ARIAD] is used. Examples of these dimerizer molecules that can be used in the present invention include, but are not limited to rapamycin. FK506, FK10¹² (a homodimer of FK506), rapamycin analogs (“rapalogs”) which are readily prepared by chemical modifications of the natural product to add a “bump” that reduces or eliminates affinity for endogenous FKBP and/or FRAP. Examples of rapalogs include, but are not limited to such as AP26113 (Ariad), AP1510 (Amara, J. F., et al., 1997, Proc Natl Acad Sci USA, 94(20): 10618-23) AP22660, AP22594, AP21370, AP22594, AP23054, AP1855, AP1856, AP1701, AP1861, AP1692 and AP1889, with designed ‘bumps’ that minimize interactions with endogenous FKBP. Still other rapalogs may be selected, e.g., AP23573 [Merck]. In certain embodiments, rapamycin or a suitable analog may be delivered locally to the AAV-transfected cells of the nasopharynx. This local delivery may be by intranasal injection, topically to the cells via bolus, cream, or gel. See, US Patent Application US 2019/0216841 A1, which is incorporated herein by reference.

Other suitable enhancers include those that are appropriate for a desired target tissue indication. In one embodiment, the expression cassette comprises one or more expression enhancers. In one embodiment, the expression cassette contains two or more expression enhancers. These enhancers may be the same or may differ from one another. For example, an enhancer may include a CMV immediate early enhancer. This enhancer may be present in two copies which are located adjacent to one another. Alternatively, the dual copies of the enhancer may be separated by one or more sequences. In still another embodiment, the expression cassette further contains an intron, e.g., the chicken beta-actin intron. Other suitable introns include those known in the art, e.g., such as are described in WO 2011/126808. Examples of suitable polyA sequences include, e.g., rabbit beta globin (rBG), SV40, SV50, bovine growth hormone (bGH), human growth hormone, and synthetic polyAs. Optionally, one or more sequences may be selected to stabilize mRNA. An example of such a sequence is a modified WPRE sequence, which may be engineered upstream of the polyA sequence and downstream of the coding sequence. Suitable WPRE sequences are provided in the vector genomes described herein and are known in the art (e.g., such as those are described in U.S. Pat. Nos. 6,136,597, 6,287,814, and 7,419,829, which are incorporated by reference). In certain embodiments, the WPRE is a variant that has been mutated to eliminate expression of the woodchuck hepatitis B virus X (WHX) protein, including, for example, mutations in the start codon of the WHX gene. See also, Kingsman S. M., Mitrophanous K., & Olsen J. C. (2005), Potential Oncogene Activity of the Woodchuck Hepatitis Post-Transcriptional Regulatory Element (Wpre).” Gene Ther. 12(1):3-4; and Zanta-Boussif M. A., Charrier S., Brice-Ouzet A., Martin S., Opolon P., Thrasher A. J., Hope T. J., & Galy A. (2009), Validation of a Mutated Pre-Sequence Allowing High and Sustained Transgene Expression While Abrogating Whv-X Protein Synthesis: Application to the Gene Therapy of Was, Gene Ther. 16(5):605-19, both of which are incorporated herein by reference in its entirety. See also, SEQ ID NO: 13 (WPRE element mutated). In other embodiments, enhancers are selected from a non-viral source. In certain embodiments, no WPRE sequence is present.

An AAV viral vector may include multiple transgenes. In certain embodiments, the transgene may be used to correct or ameliorate gene deficiencies, which may include deficiencies in which normal genes are expressed at less than normal levels or deficiencies in which the functional gene product is not expressed. Alternatively, the transgene may provide a product to a cell which is not natively expressed in the cell type or in the host. A preferred type of transgene sequence encodes a therapeutic protein or polypeptide which is expressed in a target cell. The invention further includes using multiple transgenes. In certain situations, a different transgene may be used to encode each subunit of a protein, or to encode different peptides or proteins. This is desirable when the size of the DNA encoding the protein subunit is large, e.g., for an immunoglobulin, the platelet-derived growth factor, or a dystrophin protein. In certain situations, a different transgene may be used to encode each subunit of a protein (e.g., an immunoglobulin domain, an immunoglobulin heavy chain, an immunoglobulin light chain). In one embodiment, a target cell produces the multi-subunit protein following infected/transfection with the virus containing each of the different subunits. In another embodiment, different subunits of a protein may be encoded by the same transgene. In certain embodiments, an IRES or self-cleaving enzyme (2A) is desirable when the size of the DNA encoding each of the subunits is small, e.g., the total size of the DNA encoding the subunits. Typically, an IRES is less than five kilobases. As an alternative to an IRES, the DNA may be separated by sequences encoding a 2A peptide, which self-cleaves in a post-translational event. See, e.g., ML Donnelly, et al, (January 1997) J. Gen. Virol., 78(Pt 1):13-21; S. Furler, S et al, (June 2001) Gene Ther., 8(11):864-873; H. Klump, et al., (May 2001) Gene Ther., 8(10):811-817. This 2A peptide is significantly smaller than IRES, making it well suited for use when space is a limiting factor. More often, when the transgene is large, consists of multi-subunits, or two transgenes are co-delivered, rAAV carrying the desired transgene(s) or subunits are co-administered to allow them to concatamerize in vivo to form a single vector genome. In such an embodiment, a first AAV may carry an expression cassette which expresses a single transgene and a second AAV may carry an expression cassette which expresses a different transgene for co-expression in the target cell. However, the selected transgene may encode any biologically active product or other product, e.g., a product desirable for study.

In addition to the elements identified above for the expression cassette, the vector also includes conventional control elements which are operably linked to the coding sequence in a manner which permits transcription, translation and/or expression of the encoded product in a cell transfected with the plasmid vector or infected with the virus produced by the invention. Examples of suitable transgenes are provided herein. As used herein, “operably linked” sequences include both expression control sequences that are contiguous with the gene of interest and expression control sequences that act in trans or at a distance to control the gene of interest.

Expression control sequences include appropriate enhancer; transcription factor; transcription terminator; promoter; efficient RNA processing signals such as splicing and polyadenylation (polyA) signals; sequences that stabilize cytoplasmic mRNA, for example Woodchuck Hepatitis Virus (WHP) Posttranscriptional Regulatory Element (WPRE); sequences that enhance translation efficiency (i.e., Kozak consensus sequence); sequences that enhance protein stability; and when desired, sequences that enhance secretion of the encoded product.

In one embodiment, the regulatory sequences are selected such that the total rAAV vector genome is about 2.0 to about 5.5 kilobases in size. In one embodiment, it is desirable that the rAAV vector genome approximate the size of the native AAV genome. Thus, in one embodiment, the regulatory sequences are selected such that the total rAAV vector genome is about 4.7 kb in size. In another embodiment, the total rAAV vector genome is less about 5.2 kb in size. The size of the vector genome may be manipulated based on the size of the regulatory sequences including the promoter, enhancer, intron, poly A, etc. See, Wu et al, Mol Ther, Jan. 2010 18(1):80-6, which is incorporated herein by reference.

Thus, in one embodiment, an intron is included in the vector. Suitable introns include chicken beta-actin intron, the human beta globin IVS2 (Kelly et al, Nucleic Acids Research, 43(9):4721-32 (2015)); the Promega chimeric intron (Almond, B. and Schenborn, E. T. A Comparison of pCI-neo Vector and pcDNA4/HisMax Vector); and the hFIX intron. Various introns suitable herein are known in the art and include, without limitation, those found at bpg.utoledo.edu/˜afedorov/lab/eid, which is incorporated herein by reference. See also, Shepelev V., Fedorov A. Advances in the Exon-Intron Database. Briefings in Bioinformatics 2006, 7: 178-185, which is incorporated herein by reference.

Several different viral genomes were generated in the studies described herein. However, it will be understood by the skilled artisan that other genomic configurations, including other regulatory sequences may be substituted for the promoter, enhancer and other coding sequences may be selected.

rAAV Production

For use in producing an AAV viral vector (e.g., a recombinant (r) AAV), the expression cassettes can be carried on any suitable vector, e.g., a plasmid, which is delivered to a production (packaging) host cell. The plasmids useful in this invention may be engineered such that they are suitable for replication and packaging in vitro in prokaryotic cells, insect cells, mammalian cells, among others. In certain embodiments, the production host cell is a human cell or insect cell. In certain embodiments, the production host cell in is HEK293 cell, HuH-7 cell, BHK cell, or Vero cell. In certain embodiments, production host cell is in a suspension cell culture. Suitable transfection techniques and packaging host cells are known and/or can be readily designed by one of skill in the art.

In certain embodiments, provided herein is a production host cell comprising a recombinant nucleic acid molecule as described herein, a nucleic acid sequence encoding an AAV capsid protein, and sufficient AAV rep functions and helper functions to permit packaging of the vector genome into the AAV capsid.

In some embodiments, the use of an amino acid sequence for AAVhu95 capsid, encoded by the nucleic acid sequence of SEQ ID NO: 1 or a sequence 91% to 100% identical, at least 95% to 99% identical, at least 97% identical, at least 98% identical, or at least 99% identical, in rAAV comprising AAVhu95 capsid provides advantages in production, wherein the production host cells are 293 (HEK293) cells. In other embodiments, the use of an amino acid sequence for AAVhu96 capsid, encoded by the nucleic acid sequence of SEQ ID NO: 3 or a sequence 91% to 100% identical, at least 95% to 99% identical, at least 97% identical, at least 98% identical, at least 99% identical, to SEQ ID NO: 3, in rAAV comprising AAVhu96 capsid provides advantages in production, wherein the production cells are 293 cells.

Methods of preparing AAV-based vectors (e.g., having an AAV9 or another AAV capsid) are known. See, e.g., US Published Patent Application No. 2007/0036760 (Feb. 15, 2007), which is incorporated by reference herein. The invention is not limited to the use of AAV9 or other clade F AAV amino acid sequences, but encompasses peptides and/or proteins containing the terminal β-galactose binding generated by other methods known in the art, including, e.g., by chemical synthesis, by other synthetic techniques, or by other methods. The sequences of any of the AAV capsids provided herein can be readily generated using a variety of techniques. Suitable production techniques are well known to those of skill in the art. See, e.g., Sambrook et al, Molecular Cloning: A Laboratory Manual, Cold Spring Harbor Press (Cold Spring Harbor, NY). Alternatively, peptides can also be synthesized by the well-known solid phase peptide synthesis methods (Merrifield, (1962) J. Am. Chem. Soc., 85:2149; Stewart and Young, Solid Phase Peptide Synthesis (Freeman, San Francisco, 1969) pp. 27-62). These methods may involve, e.g., culturing a host cell which contains a nucleic acid sequence encoding an AAV capsid; a functional rep gene; a minigene composed of, at a minimum, AAV inverted terminal repeats (ITRs) and a transgene; and sufficient helper functions to permit packaging of the minigene into the AAV capsid protein. These and other suitable production methods are within the knowledge of those of skill in the art and are not a limitation of the present invention.

The components required to be cultured in the host cell to package an AAV minigene in an AAV capsid may be provided to the host cell in trans. Alternatively, any one or more of the required components (e.g., minigene, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contains the rep and/or cap proteins under the control of inducible promoters. Still other stable host cells may be generated by one of skill in the art.

These rAAVs are particularly well suited to gene delivery for therapeutic purposes and for preventing infection. Further, the compositions of the invention may also be used for production of a desired gene product in vitro. For in vitro production, a desired product (e.g., a protein) may be obtained from a desired culture following transfection of host cells with a rAAV containing the molecule encoding the desired product and culturing the cell culture under conditions which permit expression. The expressed product may then be purified and isolated, as desired. Suitable techniques for transfection, cell culturing, purification, and isolation are known to those of skill in the art. Methods for generating and isolating AAVs suitable for use as vectors are known in the art. See generally, e.g., Grieger & Samulski, 2005, “Adeno-associated virus as a gene therapy vector: Vector development, production and clinical applications,” Adv. Biochem. Engin/Biotechnol. 99: 119-145; Buning et al., 2008, “Recent developments in adeno-associated virus vector technology,” J. Gene Med. 10:717-733; and the references cited below, each of which is incorporated herein by reference in its entirety. For packaging a transgene into virions, the ITRs are the only AAV components required in cis in the same construct as the nucleic acid molecule containing the expression cassettes. The cap and rep genes can be supplied in trans.

In one embodiment, the expression cassettes described herein are engineered into a genetic element (e.g., a shuttle plasmid) which transfers the immunoglobulin construct sequences carried thereon into a packaging host cell for production a viral vector. In one embodiment, the selected genetic element may be delivered to an AAV packaging cell by any suitable method, including transfection, electroporation, liposome delivery, membrane fusion techniques, high velocity DNA-coated pellets, viral infection and protoplast fusion. Stable AAV packaging cells can also be made. Alternatively, the expression cassettes may be used to generate a viral vector other than AAV, or for production of mixtures of antibodies in vitro. The methods used to make such constructs are known to those with skill in nucleic acid manipulation and include genetic engineering, recombinant engineering, and synthetic techniques. See, e.g., Molecular Cloning: A Laboratory Manual, ed. Green and Sambrook, Cold Spring Harbor Press, Cold Spring Harbor, NY (2012).

The term “AAV intermediate” or “AAV vector intermediate” refers to an assembled rAAV capsid which lacks the desired genomic sequences packaged therein. These may also be termed an “empty” capsid. Such a capsid may contain no detectable genomic sequences of an expression cassette, or only partially packaged genomic sequences which are insufficient to achieve expression of the gene product. These empty capsids are non-functional to transfer the gene of interest to a host cell.

The recombinant AAV described herein may be generated using techniques which are known. See, e.g., WO 2003/042397; WO 2005/033321, WO 2006/110689; U.S. Pat. No. 7,588,772 B2. Such a method involves culturing a production host cell which contains a nucleic acid sequence encoding an AAV capsid; a functional rep gene; an expression cassette composed of, at a minimum, AAV inverted terminal repeats (ITRs) and a transgene; and sufficient helper functions to permit packaging of the expression cassette into the AAV capsid protein. Methods of generating the capsid, coding sequences therefore, and methods for production of rAAV viral vectors have been described. See, e.g., Gao, et al, Proc. Natl. Acad. Sci. U.S.A. 100 (10), 6081-6086 (2003) and US 2013/0045186A1.

In one embodiment, cells are manufactured in a suitable production cell culture (e.g., HEK 293 cells). Methods for manufacturing the gene therapy vectors described herein include methods well known in the art such as generation of plasmid DNA used for production of the gene therapy vectors, generation of the vectors, and purification of the vectors. In some embodiments, the gene therapy vector is an AAV vector and the plasmids generated are an AAV cis-plasmid encoding the AAV genome and the gene of interest, an AAV trans-plasmid containing AAV rep and cap genes, and an adenovirus helper plasmid. The vector generation process can include method steps such as initiation of cell culture, passage of cells, seeding of cells, transfection of cells with the plasmid DNA, post-transfection medium exchange to serum free medium, and the harvest of vector-containing cells and culture media. The harvested vector-containing cells and culture media are referred to herein as crude cell harvest. In yet another system, the gene therapy vectors are introduced into insect cells by infection with baculovirus-based vectors. For reviews on these production systems, see generally, e.g., Zhang et al., 2009, “Adenovirus-adeno-associated virus hybrid for large-scale recombinant adeno-associated virus production,” Human Gene Therapy 20:922-929, which is incorporated herein by reference in its entirety. Methods of making and using these and other AAV production systems are also described in the following U.S. patents, the contents of each of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065. See also, WO2017/160360 A2, which is incorporated herein by reference in its entirety.

The crude cell harvest may thereafter be subject method steps such as concentration of the vector harvest, diafiltration of the vector harvest, microfluidization of the vector harvest, nuclease digestion of the vector harvest, filtration of microfluidized intermediate, crude purification by chromatography, crude purification by ultracentrifugation, buffer exchange by tangential flow filtration, and/or formulation and filtration to prepare bulk vector. An affinity chromatography purification followed anion exchange resin chromatography are used to purify the vector drug product and to remove empty capsids. These methods are described in more detail in WO2017/160360, filed Dec. 9, 2016, entitled “Scalable Purification Method for AAV9”, which is incorporated by reference. Purification methods for AAV8, WO2017/100676, filed Dec. 9, 2016, and rh10, WO2017/100704, filed Dec. 9, 2016, entitled “Scalable Purification Method for AAVrh10”, also filed Dec. 11, 2015, and for AAV1, WO2017/100674, filed Dec. 9, 2016 for “Scalable Purification Method for AAV1”, filed Dec. 11, 2015, are all incorporated by reference herein. Other suitable methods may be selected.

Conventional methods for characterization or quantification of rAAV are available to one of skill in the art. Methods for determining the ratio among vp1, vp2, and vp3 of capsid protein are also available. See, e.g., Vamseedhar Rayaprolu et al., Comparative Analysis of Adeno-Associated Virus Capsid Stability and Dynamics, J Virol. 2013 December; 87(24): 13150-13160; Buller R M, Rose J A. 1978. Characterization of adenovirus-associated virus-induced polypeptides in KB cells. J. Virol. 25:331-338; and Rose J A, Maizel J V, Inman J K, Shatkin A J. 1971. Structural proteins of adenovirus-associated viruses. J. Virol. 8:766-770.

To calculate empty and full particle content, vp3 band volumes for a selected sample (e.g., in examples herein an iodixanol gradient-purified preparation where number of GC=number of particles) are plotted against GC particles loaded. The resulting linear equation (y=mx+c) is used to calculate the number of particles in the band volumes of the test article peaks. The number of particles (pt) per 20 μL loaded is then multiplied by 50 to give particles (pt)/mL. Pt/mL divided by GC/mL gives the ratio of particles to genome copies (pt/GC). Pt/mL-GC/mL gives empty pt/mL. Empty pt/mL divided by pt/mL and ×100 gives the percentage of empty particles.

Generally, methods for assaying for empty capsids and AAV vector particles with packaged genomes have been known in the art. See, e.g., Grimm et al., Gene Therapy (1999) 6:1322-1330; and Sommer et al., Molec. Ther. (2003) 7:122-128. To test for denatured capsid, the methods include subjecting the treated AAV stock to SDS-polyacrylamide gel electrophoresis, consisting of any gel capable of separating the three capsid proteins, for example, a gradient gel containing 3-8% Tris-acetate in the buffer, then running the gel until sample material is separated, and blotting the gel onto nylon or nitrocellulose membranes, preferably nylon. Anti-AAV capsid antibodies are then used as the primary antibodies that bind to denatured capsid proteins, preferably an anti-AAV capsid monoclonal antibody, most preferably the B1 anti-AAV-2 monoclonal antibody (Wobus et al., J. Virol. (2000) 74:9281-9293). A secondary antibody is then used, one that binds to the primary antibody and contains a means for detecting binding with the primary antibody, more preferably an anti-IgG antibody containing a detection molecule covalently bound to it, most preferably a sheep anti-mouse IgG antibody covalently linked to horseradish peroxidase. A method for detecting binding is used to semi-quantitatively determine binding between the primary and secondary antibodies, preferably a detection method capable of detecting radioactive isotope emissions, electromagnetic radiation, or colorimetric changes, most preferably a chemiluminescence detection kit. For example, for SDS-PAGE, samples from column fractions can be taken and heated in SDS-PAGE loading buffer containing reducing agent (e.g., DTT), and capsid proteins were resolved on pre-cast gradient polyacrylamide gels (e.g., Novex). Silver staining may be performed using SilverXpress (Invitrogen, CA) according to the manufacturer's instructions or other suitable staining method, i.e., SYPRO ruby or coomassie stains. In one embodiment, the concentration of AAV vector genomes (vg) in column fractions can be measured by quantitative real time PCR (Q-PCR). Samples are diluted and digested with DNase I (or another suitable nuclease) to remove exogenous DNA. After inactivation of the nuclease, the samples are further diluted and amplified using primers and a TaqMan™ fluorogenic probe specific for the DNA sequence between the primers. The number of cycles required to reach a defined level of fluorescence (threshold cycle, Ct) is measured for each sample on an Applied Biosystems Prism 7700 Sequence Detection System. Plasmid DNA containing identical sequences to that contained in the AAV vector is employed to generate a standard curve in the Q-PCR reaction. The cycle threshold (Ct) values obtained from the samples are used to determine vector genome titer by normalizing it to the Ct value of the plasmid standard curve. End-point assays based on the digital PCR can also be used.

Additionally, another example of measuring empty to full particle ratio is also known in the art. Sedimentation velocity, as measured in an analytical ultracentrifuge (AUC) can detect aggregates, other minor components as well as providing good quantitation of relative amounts of different particle species based upon their different sedimentation coefficients. This is an absolute method based on fundamental units of length and time, requiring no standard molecules as references. Vector samples are loaded into cells with 2-channel charcoal-epon centerpieces with 12 mm optical path length. The supplied dilution buffer is loaded into the reference channel of each cell. The loaded cells are then placed into an AN-60Ti analytical rotor and loaded into a Beckman-Coulter ProteomeLab XL-I analytical ultracentrifuge equipped with both absorbance and RI detectors. After full temperature equilibration at 20° C. the rotor is brought to the final run speed of 12,000 rpm. A280 scans are recorded approximately every 3 minutes for ˜5.5 hours (110 total scans for each sample). The raw data is analyzed using the c(s) method and implemented in the analysis program SEDFIT. The resultant size distributions are graphed and the peaks integrated. The percentage values associated with each peak represent the peak area fraction of the total area under all peaks and are based upon the raw data generated at 280 nm; many labs use these values to calculate empty: full particle ratios. However, because empty and full particles have different extinction coefficients at this wavelength, the raw data can be adjusted accordingly. The ratio of the empty particle and full monomer peak values both before and after extinction coefficient-adjustment is used to determine the empty-full particle ratio.

In one aspect, an optimized q-PCR method is used which utilizes a broad spectrum serine protease, e.g., proteinase K (such as is commercially available from Qiagen). More particularly, the optimized qPCR genome titer assay is similar to a standard assay, except that after the DNase I digestion, samples are diluted with proteinase K buffer and treated with proteinase K followed by heat inactivation. Suitably samples are diluted with proteinase K buffer in an amount equal to the sample size. The proteinase K buffer may be concentrated to 2-fold or higher. Typically, proteinase K treatment is about 0.2 mg/mL, but may be varied from 0.1 mg/mL to about 1 mg/mL. The treatment step is generally conducted at about 55° C. for about 15 minutes, but may be performed at a lower temperature (e.g., about 37° C. to about 50° C.) over a longer time period (e.g., about 20 minutes to about 30 minutes), or a higher temperature (e.g., up to about 60° C.) for a shorter time period (e.g., about 5 to 10 minutes). Similarly, heat inactivation is generally at about 95° C. for about 15 minutes, but the temperature may be lowered (e.g., about 70 to about 90° C.) and the time extended (e.g., about 20 minutes to about 30 minutes). Samples are then diluted (e.g., 1000-fold) and subjected to TaqMan analysis as described in the standard assay. Quantification also can be done using ViroCyt or flow cytometry.

Additionally, or alternatively, droplet digital PCR (ddPCR) may be used. For example, methods for determining single-stranded and self-complementary AAV vector genome titers by ddPCR have been described. See, e.g., M. Lock et al, Hu Gene Therapy Methods, Hum Gene Ther Methods. 2014 April; 25(2):115-25. doi: 10.1089/hgtb.2013.131. Epub 2014 Feb. 14.

In certain embodiments, the manufacturing process for rAAVhu95 or rAAVhu96 involves method as described in U.S. Provisional Patent Application No. 63/371,597, filed Aug. 16, 2022, and US Provisional Patent Application No. 63/371,592, filed Aug. 16, 2022, which are incorporated herein by reference in its entirety.

As described herein, a rAAVhu95 has a rAAVhu95 capsid produced in a production system expressing capsids from AAVhu95 nucleic acid sequences which encode the vp1 amino acid sequence of SEQ ID NO: 2, and optionally additional nucleic acid sequences, e.g., encoding a vp3 protein free of the vp1 and/or vp2-unique regions. In certain embodiments, the nucleic acid sequence used in the production system, and which encode the AAVhu95 capsid, is selected from SEQ ID NO: 1, or a sequence 91% to 100% identical, or 95% to 99.9% identical to SEQ ID NO: 1, or SEQ ID NO: 10 or a sequence at least 99% identical to SEQ ID NO: 10. The rAAVhu95 resulting from production using a single nucleic acid sequence vp1 produces the heterogeneous populations of vp1 proteins, vp2 proteins and vp3 proteins. More particularly, the AAVhu95 capsid contains subpopulations within the vp1 proteins, within the vp2 proteins and within the vp3 proteins which have modifications from the predicted amino acid residues in SEQ ID NO: 2.

Additionally, as described herein, a rAAVhu96 has a rAAVhu96 capsid produced in a production system expressing capsids from AAVhu96 nucleic acid sequences which encode the vp1 amino acid sequence of SEQ ID NO: 4, and optionally additional nucleic acid sequences. e.g., encoding a vp3 protein free of the vp1 and/or vp2-unique regions. In certain embodiments, the nucleic acid sequence used in the production system, and which encode the AAVhu96 capsid, is selected from SEQ ID NO: 3, or a sequence 91% to 100% identical, or 95% to 99.9% identical to SEQ ID NO: 3, or SEQ ID NO: 11 or a sequence at least 99% identical to SEQ ID NO: 11. The rAAVhu96 resulting from production using a single nucleic acid sequence vp1 produces the heterogeneous populations of vp1 proteins, vp2 proteins and vp3 proteins. More particularly, the AAVhu96 capsid contains subpopulations within the vp1 proteins, within the vp2 proteins and within the vp3 proteins which have modifications from the predicted amino acid residues in SEQ ID NO: 4.

It should be understood that the compositions in the vectors described herein are intended to be applied to other compositions and methods described across the specification.

### Compositions, Methods and Uses

Provided herein are compositions containing at least one rAAV stock (e.g., an rAAVhu95 or rAAVhu96) and an optional carrier, excipient and/or preservative. An rAAV stock refers to a plurality of rAAV vectors which are the same, e.g., such as in the amounts described below in the discussion of concentrations and dosage units.

In one aspect, provided herein is a method of delivering of a transgene to one or more target cells of the central nervous system (CNS), motor neurons, or another targeted cell type, of a subject comprising administering to the subject a recombinant adeno-associated virus (AAV) vector comprising an AAVhu95 capsid and a vector genome comprising the transgene operably linked to regulatory sequences that direct expression of the transgene in the target cells.

In another aspect, provided herein is a method of delivering of a transgene to one or more target cells of the central nervous system (CNS), motor neurons, or another targeted cell type, of a subject comprising administering to the subject a recombinant adeno-associated virus (AAV) vector comprising an AAVhu96 capsid and a vector genome comprising the transgene operably linked to regulatory sequences that direct expression of the transgene in the target cells.

In yet another aspect, provided herein is a method of delivering of a transgene to one or more target cells of the cardiovascular (i.e., heart tissue) of a subject comprising administering to the subject a recombinant adeno-associated virus (AAV) vector comprising an AAVhu95 or AAVhu96 capsid, and a vector genome comprising the transgene operably linked to regulatory sequences that direct expression of the transgene in the target cells.

In certain embodiments, a composition may contain at least a second, different rAAV stock. This second vector stock may vary from the first by having a different AAV capsid and/or a different vector genome. In certain embodiments, a composition as described herein may contain a different vector expressing an expression cassette as described herein, or another active component (e.g., an antibody construct, another biologic, and/or a small molecule drug).

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host. Delivery vehicles such as liposomes, nanocapsules, microparticles, microspheres, lipid particles, vesicles, and the like, may be used for the introduction of the compositions of the present invention into suitable target cells. In particular, the rAAV vector delivered transgenes may be formulated for delivery either encapsulated in a lipid particle, a liposome, a vesicle, a nanosphere, or a nanoparticle or the like.

In one embodiment, a composition includes a final formulation suitable for delivery to a subject, e.g., is an aqueous liquid suspension buffered to a physiologically compatible pH and salt concentration. Suitably, the final formulation is adjusted to a physiologically acceptable pH, e.g., the pH may be in the range of pH 6 to 9, or pH 6.5 to 7.5, pH 7.0 to 7.7, or pH 7.2 to 7.8. As the pH of the cerebrospinal fluid is about 7.28 to about 7.32, for intrathecal delivery, a pH within this range may be desired; whereas for intravenous delivery, a pH of 6.8 to about 7.2 may be desired. However, other pHs within the broadest ranges and these subranges may be selected for other routes of delivery. Optionally, one or more surfactants are present in the formulation. In another embodiment, the composition may be transported as a concentrate which is diluted for administration to a subject. In other embodiments, the composition may be lyophilized and reconstituted at the time of administration.

A suitable surfactant, or combination of surfactants, may be selected from among nonionic surfactants that are nontoxic. In one embodiment, a difunctional block copolymer surfactant terminating in primary hydroxyl groups is selected, e.g., such as Pluronic® F68 [BASF], also known as Poloxamer 188, which has a neutral pH, has an average molecular weight of 8400. Other surfactants and other Poloxamers may be selected, i.e., nonionic triblock copolymers composed of a central hydrophobic chain of polyoxypropylene (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polvoxy 10 oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits×100 give the approximate molecular mass of the polyoxypropylene core, and the last digit×10 gives the percentage polyoxyethylene content. In one embodiment Poloxamer 188 is selected. The surfactant may be present in an amount up to about 0.0005% to about 0.001% of the suspension.

In one embodiment, the compositions described herein are used in preparing medicaments for treating central nervous system disorders and diseases. Optionally, the compositions described herein are administered in the absence of an additional extrinsic pharmacological or chemical agent, or other physical disruption of the blood brain barrier.

In one embodiment, the formulation buffer is phosphate-buffered saline (PBS) with total salt concentration of 200 mM, 0.001% (w/v) Pluronic F68 (Final Formulation Buffer, FFB).

In certain embodiments, the composition comprises a viral vector (i.e., rAAV vector). The vectors are administered in sufficient amounts to transfect the cells and to provide sufficient levels of gene transfer and expression to provide a therapeutic benefit without undue adverse effects, or with medically acceptable physiological effects, which can be determined by those skilled in the medical arts. In certain embodiments, the vectors are formulated for delivery via intranasal delivery devices. In certain embodiments, vectors are formulated for aerosol delivery devices, e.g., via a nebulizer or through other suitable devices. In certain embodiment, vectors are formulated for intrathecal delivery. In some embodiments, intrathecal delivery encompasses an injection into the spinal canal, e.g., the subarachnoid space. In some embodiments, other delivery route may be selected, e.g., intracranial, intranasal, intracisternal, intracerebrospinal fluid delivery, among other suitable direct or systemic routes, i.e., Ommaya reservoir. In certain embodiments, intrathecal delivery comprises the steps of CT-guided sub-occipital injection via spinal needle into the cisterna magna of a patient. As used herein, the term Computed Tomography (CT) refers to radiography in which a three-dimensional image of a body structure is constructed by computer from a series of plane cross-sectional images made along an axis. In certain embodiments, the apparatus is described in US Patent Publication No. 2018-0339065 A1, published Nov. 29, 2019, which is incorporated herein by reference in its entirety. In certain embodiments, vectors are formulated for intravenous delivery. Other conventional and pharmaceutically acceptable routes of administration include, but are not limited to, direct delivery to a desired organ (e.g., lung), oral inhalation, intratracheal, intraarterial, intraocular, intravenous, intramuscular, subcutaneous, intradermal, and other parenteral routes of administration. In one embodiment, the vector is administered intranasally using intranasal mucosal atomization device (LMA® MAD Nasal™—MAD110). In another embodiment the vector is administered intrapulmonary in nebulized form using Vibrating Mesh Nebulizer (Aerogen® Solo) or MADgic™ Laryngeal Mucosal Atomizer. Routes of administration may be combined, if desired. Routes of administration and utilization of which for delivering rAAV vectors are also described in the following published US Patent Applications, the contents of each of which is incorporated herein by reference in its entirety: US 2018/0155412A1, US 2018/0243416A1, US 2014/0031418 A1, and US 2019/0216841A1.

In certain embodiments, vectors are formulated for delivery to central nervous system using image-guided direct injection. In certain embodiments, the image-guided direct injection (e.g., substantia nigra ventral tegmental area) is an MRI-guided convection-enhanced injection. In certain embodiments, a convection-enhanced delivery (CED) refers to the use of a pressure gradient to generate bulk flow within the brain parenchyma, i.e., convection of composition within the interstitial fluid driven by infusing a solution through a cannula placed directly in the targeted structure. This method allows therapeutic agents to be homogenously distributed through large volumes of brain tissue by bypassing the blood brain barrier and surpassing simple diffusion (Richardson, et al., 2011, Novel Platform for MmU-Guided Convection-Enhanced Delivery of Therapeutics: Preclinical Validation in Nonhuman Primate Brain, Stereotact. Funct. Neurosurg. 89(3):141-151, which is incorporated herein by reference in its entirety). See also, Kalkowski, L., et al., 2018, MRI-guided intracerebral convection-enhanced injection of gliotoxins to induce focal demyelination in swine, PLOS One, 13(10): e0204650; WO2016073693A2; and Prezelski, K., et al., 2021, Design and Validation of a Multi-Point Injection Technology for MR-Guided Convection Enhanced Delivery in the Brain, Front. Med. Technol., 14(3):725844, which are incorporated herein by reference in their entireties.

Dosages of the viral vector will depend primarily on factors such as the condition being treated, the age, weight and health of the patient, and may thus vary among patients. For example, a therapeutically effective human dosage of the viral vector is generally in the range of from about 25 to about 1000 microliters to about 5 mL of aqueous suspending liquid containing doses of from about 10⁹ to 4×10¹⁴ GC of AAV vector. The dosage will be adjusted to balance the therapeutic benefit against any side effects and such dosages may vary depending upon the therapeutic application for which the recombinant vector is employed. The levels of expression of the transgene can be monitored to determine the frequency of dosage resulting in viral vectors, preferably AAV vectors containing the minigene. Optionally, dosage regimens similar to those described for therapeutic purposes may be utilized for immunization using the compositions of the invention.

The replication-defective virus compositions can be formulated in dosage units to contain an amount of replication-defective virus that is in the range of about 10′ GC to about 10¹⁶ GC (to treat an average subject of 70 kg in body weight) including all integers or fractional amounts within the range, and preferably 10¹² GC to 10¹⁴ GC for a human patient. In one embodiment, the compositions are formulated to contain at least 10, 2×10⁹, 3×10⁹, 4×10⁹, 5×10⁹, 6×10⁹, 7×10⁹, 8×10⁹, or 9×10⁹ GC per dose including all integers or fractional amounts within the range. In another embodiment, the compositions are formulated to contain at least 10¹⁰, 2×10¹⁰, 3×10¹⁰, 4×10¹⁰, 5×10¹⁰, 6×10¹⁰, 7×10¹⁰, 8×10¹⁰, or 9×10¹⁰ GC per dose including all integers or fractional amounts within the range. In another embodiment, the compositions are formulated to contain at least 10¹¹, 2×10¹¹, 3×10¹¹, 4×10¹¹, 5×10¹¹, 6×10¹¹, 7×10¹¹, 8×10¹¹, or 9×10¹¹ GC per dose including all integers or fractional amounts within the range. In another embodiment, the compositions are formulated to contain at least 10¹², 2×10¹², 3×10¹², 4×10¹², 5×10¹², 6×10¹² 7×10¹², 8×10¹², or 9×10¹² GC per dose including all integers or fractional amounts within the range. In another embodiment, the compositions are formulated to contain at least 10¹³, 2×10¹³, 3×10¹³, 4×10¹³, 5×10¹³, 6×10¹³, 7×10¹³, 8×10¹³, or 9×10¹³ GC per dose including all integers or fractional amounts within the range. In another embodiment, the compositions are formulated to contain at least 10¹⁴, 2×10¹⁴, 3×10¹⁴, 4×10⁴, 5×10¹⁴, 6×10¹⁴, 7×10¹⁴, 8×10¹⁴, or 9×10¹⁴ GC per dose including all integers or fractional amounts within the range. In another embodiment, the compositions are formulated to contain at least 10¹⁵, 2×10¹⁵, 3×10¹⁵, 4×10¹⁵, 5×10¹⁵, 6×10¹⁵, 7×10¹⁵, 8×10¹⁵, or 9×10¹⁵ GC per dose including all integers or fractional amounts within the range. In one embodiment, for human application the dose can range from 10¹⁰ to about 10¹² GC per dose including all integers or fractional amounts within the range. In one embodiment, for human application the dose can range from 10⁹ to about 7×10¹³ GC per dose including all integers or fractional amounts within the range. In one embodiment, for human application the dose ranges from 6.25×10¹² GC to 5.00×10³ GC. In a further embodiment, the dose is about 6.25×10¹² GC, about 1.25×10¹³ GC, about 2.50×10¹³ GC, or about 5.00×10¹³ GC. In certain embodiment, the dose is divided into one half thereof equally and administered to each nostril. In certain embodiments, for human application the dose ranges from 6.25×10² GC to 5.00×10³ GC administered as two aliquots of 0.2 ml per nostril for a total volume delivered in each subject of 0.8 ml.

These above doses may be administered in a variety of volumes of carrier, excipient or buffer formulation, ranging from about 25 to about 1000 microliters, or higher volumes, including all numbers within the range, depending on the size of the area to be treated, the viral titer used, the route of administration, and the desired effect of the method. In one embodiment, the volume of carrier, excipient or buffer is at least about 25 μL. In one embodiment, the volume is about 50 μL. In another embodiment, the volume is about 75 μL. In another embodiment, the volume is about 100 μL. In another embodiment, the volume is about 125 μL. In another embodiment, the volume is about 150 μL. In another embodiment, the volume is about 175 μL. In yet another embodiment, the volume is about 200 μL. In another embodiment, the volume is about 225 μL. In yet another embodiment, the volume is about 250 μL. In yet another embodiment, the volume is about 275 μL. In yet another embodiment, the volume is about 300 μL. In yet another embodiment, the volume is about 325 μL. In another embodiment, the volume is about 350 μL. In another embodiment, the volume is about 375 μL. In another embodiment, the volume is about 400 μL. In another embodiment, the volume is about 450 μL. In another embodiment, the volume is about 500 μL. In another embodiment, the volume is about 550 μL. In another embodiment, the volume is about 600 μL. In another embodiment, the volume is about 650 μL. In another embodiment, the volume is about 700 μL. In another embodiment, the volume is between about 700 and 1000 μL.

In certain embodiments, the dose may be in the range of about 1×10⁹ GC/g brain mass to about 1×10¹² GC/g brain mass. In certain embodiments, the dose may be in the range of about 3×10¹⁰ GC/g brain mass to about 3×10¹² GC/g brain mass. In certain embodiments, the dose may be in the range of about 5×10¹⁰ GC/g brain mass to about 1.85 ×10n GC/g brain mass.

In one embodiment, the viral constructs may be delivered in doses of from at least about least 1×10⁹ GCs to about 1×10¹⁵, or about 1×10¹¹ to 5×10¹³ GC. Suitable volumes for delivery of these doses and concentrations may be determined by one of skill in the art. For example, volumes of about 1 μL to 150 mL may be selected, with the higher volumes being selected for adults. Typically, for newborn infants a suitable volume is about 0.5 mL to about 10 mL, for older infants, about 0.5 mL to about 15 mL may be selected. For toddlers, a volume of about 0.5 mL to about 20 mL may be selected. For children, volumes of up to about 30 mL may be selected. For pre-teens and teens, volumes up to about 50 mL may be selected. In still other embodiments, a patient may receive an intrathecal administration in a volume of about 5 mL to about 15 mL are selected, or about 7.5 mL to about 10 mL. Other suitable volumes and dosages may be determined. The dosage will be adjusted to balance the therapeutic benefit against any side effects and such dosages may vary depending upon the therapeutic application for which the recombinant vector is employed.

The compositions according to the present invention may comprise a pharmaceutically acceptable carrier, such as defined above. Suitably, the compositions described herein comprise an effective amount of one or more AAV suspended in a pharmaceutically suitable carrier and/or admixed with suitable excipients designed for delivery to the subject via injection, osmotic pump, intrathecal catheter, or for delivery by another device or route. In one example, the composition is formulated for intrathecal delivery.

The composition, the suspension or the pharmaceutical compositions are encompassed herein and are designed for delivery to subjects in need thereof by any suitable route or a combination of different routes. In one embodiment, the pharmaceutical composition comprises a formulation buffer suitable for delivery via intracerebroventricular (ICV), intrathecal (IT), intracisternal, administration by direct injection into the substantia nigra and/or ventral tegmental area, or intravenous (IV) routes of administration. In certain embodiments, the rAAV or the pharmaceutical composition comprises a formulation buffer suitable for intravenous, intraparenchymal (dentate nucleus), direct injection (e.g., image guided), and/or intrathecal administration to a patient in the need thereof.

As used herein, the terms “intrathecal delivery” or “intrathecal administration” refer to a route of administration for drugs via an injection into the spinal canal, more specifically into the subarachnoid space so that it reaches the cerebrospinal fluid (CSF). Intrathecal delivery may include lumbar puncture, intraventricular, intracerebroventricular (icv) suboccipital/intracisternal, and/or C1-2 puncture. For example, material may be introduced for diffusion throughout the subarachnoid space by means of lumbar puncture. In another example, injection may be into the cisterna magna (intracisternal magna; ICM). Intracisternal delivery may increase vector diffusion and/or reduce toxicity and inflammation caused by the administration. See, e.g., Christian Hinderer et al, Widespread gene transfer in the central nervous system of cynomolgus macaques following delivery of AAV9 into the cisterna magna, Mol Ther Methods Clin Dev. 2014; 1: 14051. Published online 2014 Dec. 10. doi: 10.1038/mtm.2014.51.

As used herein, the terms “intracisternal delivery” or “intracisternal administration” refer to a route of administration for drugs directly into the cerebrospinal fluid of the brain ventricles or within the cisterna magna cerebellomedularis, more specifically via a suboccipital puncture or by direct injection into the cisterna magna or via permanently positioned tube.

As used herein, the term “intraparenchymal (dentate nucleus)” or IDN refers to a route of administration of a composition directly into dentate nuclei. IDN allows for targeting of dentate nuclei and/or cerebellum. In certain embodiments, the IDN administration is performed using ClearPoint® Neuro Navigation System (MRI Interventions, Inc., Memphis, TN) and ventricular cannula, which allows for MRI-guided visualization and administration. Alternatively, other devices and methods may be selected. In one embodiment, a frozen composition is provided which contains an rAAV in a buffer solution as described herein, in frozen form. Optionally, one or more surfactants (e.g., Pluronic F68), stabilizers or preservatives is present in this composition. Suitably, for use, a composition is thawed and titrated to the desired dose with a suitable diluent, e.g., sterile saline or a buffered saline.

In certain embodiments, the rAAV or composition thereof is administered intraparenchymally with a method which comprises using the ClearPoint® injection system wherein the system consists of a monitor to visualize the brain and injection procedure in real time, a head fixation frame that is secured to the skull, and an MRI-compatible SmartFrame® (MRI Interventions Inc., Memphis, TN) trajectory device that enables MRI-guided alignment during the procedure. This system allows for the direct injection to be combined with real-time visualization of the injection tract by MRI. To enable visualization of rAAV or composition distribution, the injection material containing the vector is mixed with gadolinium, which is contrast agent (final concentration of 1-2 mM gadolinium). During the direct injection procedure, the injection cannula is placed through the ClearPoint® frame to the correct position on the skull and the frame maintains the correct trajectory. The final position of the injection cannula is confirmed using real-time MRI images, and then the rAAV or composition is injected into the parenchyma of the deep cerebellar nuclei using convection-enhanced delivery. Each subject receives administration of the rAAV or composition plus gadolinium in each dentate nucleus injected at a rate of 0.5 μL/min initially, and then at an increased rate of up to 5 μL/min based on clinician discretion during the procedure. The procedure takes approximately 5-6 hours and subjects are anesthetized for the duration of the procedure.

In some embodiments, the rAAV or composition is administered via unilateral and/or bilateral MRI guided direct injection into the deep cerebellar nuclei (DCN) via convection-enhanced delivery (CED). In certain embodiments, the rAAV or composition is delivered using Clearpoint® NeuroNavigation system and Smartflow Cannulas, adapted for DCN injection.

In certain embodiments, a rAAVhu95 and/or rAAVhu96 as described herein may be delivered in a co-therapeutic regimen which further comprises one or more other active components. In certain embodiments, the regimen may involve co-administration of an immunomodulatory component. Such an immunomodulatory regimen may include, e.g., but are not limited to immunosuppressants such as, a glucocorticoid, steroids, antimetabolites, T-cell inhibitors, a macrolide (e.g., a rapamycin or rapalog), and cytostatic agents including an alkylating agent, an anti-metabolite, a cytotoxic antibiotic, an antibody, or an agent active on immunophilin. The immune suppressant may include a nitrogen mustard, nitrosourea, platinum compound, methotrexate, azathioprine, mercaptopurine, fluorouracil, dactinomycin, an anthracycline, mitomycin C, bleomycin, mithramycin, IL-2 receptor-(CD25−) or CD3-directed antibodies, anti-IL-2 antibodies, cyclosporin, tacrolimus, sirolimus. IFN-β, IFN-γ, an opioid, or TNF-α (tumor necrosis factor-alpha) binding agent. In certain embodiments, the immunosuppressive therapy may be started prior to the gene therapy administration. Such therapy may involve co-administration of two or more drugs, the (e.g., prednelisone, micophenolate mofetil (MMF) and/or sirolimus (i.e., rapamycin)) on the same day. One or more of these drugs may be continued after gene therapy administration, at the same dose or an adjusted dose. Such therapy may be for about 1 week, about 15 days, about 30 days, about 45 days, 60 days, or longer, as needed. Still other co-therapeutics may include, e.g., anti-IgG enzymes, which have been described as being useful for depleting anti-AAV antibodies (and thus may permit administration to patients testing above a threshold level of antibody for the selected AAV capsid), and/or delivery of anti-FcRN antibodies which is described, e.g., in U.S. Provisional Patent Application No. 63/040,381, filed Jun. 17, 2020, U.S. Provisional Patent Application No. 62/135,998, filed Jan. 11, 2021, and U.S. Provisional Patent Application No. 63/152,085, filed Feb. 22, 2021 entitled “Compositions and Methods for Treatment of Gene Therapy Patients”, now published WO 2021/257668, published 23 Dec. 2021, and/or one or more of a) a steroid or combination of steroids and/or (b) an IgG-cleaving enzyme, (c) an inhibitor of Fc-IgE binding; (d) an inhibitor of Fc-IgM binding; (e) an inhibitor of Fe-IgA binding; and/or (f) gamma interferon.

In some embodiments, the recombinant AAV (i.e., rAAVhu95 and/or rAAVhu96) containing the desired transgene and promoter for use in the target cells as detailed above is optionally assessed for contamination by conventional methods and then formulated into a pharmaceutical composition intended for administration to a subject in need thereof. Such formulation involves the use of a pharmaceutically and/or physiologically acceptable vehicle or carrier, such as buffered saline or other buffers, e.g., HEPES, to maintain pH at appropriate physiological levels, and, optionally, other medicinal agents, pharmaceutical agents, stabilizing agents, buffers, carriers, adjuvants, diluents, etc. For injection, the carrier will typically be a liquid.

Examples of proteins and compounds useful in compositions provided herein and targeted delivery include the following. It will be understood that the rAAV comprise sequences encoding the selected proteins for expression in vivo.

In certain embodiments, rAAVhu95 and/or rAAVhu96 are useful in treatment of one or more of cognitive disorders and/or neurodegenerative disorders. Such disorders may include, without limitation, transmissible spongiform encephalopathies (e.g., Creutzfeld-Jacob disease), Parkinson's disease, amyotropic lateral sclerosis (ALS), multiple sclerosis, Alzheimer's Disease, Huntington disease, Canavan's disease (e.g., associated with mutations in the aspartoacylase (ASPA) gene), traumatic brain injury, spinal cord injury (ATI335, anti-nogol by Novartis), migraine (ALD403 by Alder Biopharmaceuticals; LY2951742 by Eli; RN307 by Labrys Biologics), bovine spongiform encephalopathy, Gerstmann-Straussler-Scheinker syndrome, fatal familial insomnia, kuruysosomal storage diseases, stroke, and infectious disease affecting the central nervous system.

In certain embodiments, rAAVhu95 and/or rAAVhu96 are useful in delivery of antibodies against various infections of the central nervous. Such infectious diseases may include fungal diseases such as cryptoccocal meningitis, brain abscess, spinal epidural infection caused by, e.g., Cryptococcus neoformans, Coccidioides immitis, order Mucorales, Aspergillus spp, and Candida spp; protozoal, such as toxoplasmosis, malaria, and primary amoebic meningoencepthalitis, caused by agents such as, e.g., Toxoplasma gondii, Taenia solium, Plasmodium falciparus, Spirometra mansonoides (sparaganoisis), Echinococcus spp (causing neuro hydatosis), and cerebral amoebiasis; bacterial, such as, e.g., tuberculosis, leprosy, neurosyphilis, bacterial meningitis, lyme disease (Borrelia burgdorferi), Rocky Mountain spotted fever (Rickettsia rickettsia), CNS nocardiosis (Nocardia spp), CNS tuberculosis (Mycobacterium tuberculosis), CNS listeriosis (Listeria monocytogenes), brain abscess, and neuroborreliosis; viral infections, such as, e.g., viral meningitis, Eastern equine encephalitis (EEE), St Louis encepthalitis, West Nile virus and/or encephalitis, rabies, California encephalitis virus, La Crosse encepthalitis, measles encephalitis, poliomyelitis, which may be caused by, e.g., herpes family viruses (HSV), HSV-1, HSV-2 (neonatal herpes simplex encephalitis), varicella zoster virus (VZV), Bickerstaff encephalitis, Epstein-Barr virus (EBV), cytomegalovirus (CMV, such as TCN-202 is in development by Theraclone Sciences), human herpesvirus 6 (HHV-6), B virus (herpesvirus simiae), Flavivirus encephalitis, Japanese encephalitis, Murray valley fever, JC virus (progressive multifocal leukoencephalopathy), Nipah Virus (NiV), measles (subacute sclerosing panencephalitis); and other infections, such as, e.g., subactuate sclerosing panencephalitis, progressive multifocal leukoencephalopathy; human immunodeficiency virus (acquired immunodeficiency syndrome (AIDS)); Streptococcus pyogenes and other β-hemolytic Streptococcus (e.g., Pediatric Autoimmune Neuropsychiatric Disorders Associated with Streptococcal Infection, PANDAS) and/or Syndenham's chorea, and Guillain-Barre syndrome, and prions.

In certain embodiments, rAAVhu95 and/or rAAVhu96 comprises a transgene which encodes for the MCT8 protein (SLC16A2 gene) and other compounds and are useful for treating of Allan-Herndon-Dudley disease and the symptoms thereof.

In certain embodiments, rAAVhu95 and/or rAAVhu96 comprises a transgene which encodes for the protein is selected from a disease associated with a transport defect such as, e.g., cystic fibrosis (a cystic fibrosis transmembrane regulator), alpha-1-antitrypsin (hereditary emphysema), FE (hereditary hemaochromatosis), tyrosinase (oculocutaneous albinism), Protein C (protein C deficiency), Complement C inhibitor (type I hereditary angioedema), alpha-D-galactosidase (Fabry disease), beta hexosaminidase (Tay-Sachs), sucrase-isomaltase (congenital sucrase-isomaltase deficiency), UDP-glucoronosyl-transferase (Crigler-Najjar type II), insulin receptor (diabetes mellitus), growth hormone receptor (laron syndrome), among others. Examples of other genes and proteins those associated with, e.g. spinal muscular atrophy (SMA, SMN1), Huntingdon's Disease, Rett Syndrome (e.g., methyl-CpG-binding protein 2 (MeCP2); UniProtKB—P51608), Amyotrophic Lateral Sclerosis (ALS), Duchenne Type Muscular dystrophy, Friedrichs Ataxia (e.g., frataxin), ATXN2 associated with spinocerebellar ataxia type 2 (SCA2)/ALS; TDP-43 associated with ALS, progranulin (PRGN) (associated with non-Alzheimer's cerebral degenerations, including, frontotemporal dementia (FTD), progressive non-fluent aphasia (PNFA) and semantic dementia), CDKL5 deficiency, Angelman syndrome, N-glycanase 1 deficiency, Alzheimer's disease, Fragile X syndrome, Neimann Pick disease (including types A and B (ASMD or Acid Sphingomyelinase Deficiency), and type c (NPC), mucopolysaccharidoses (MPS), Wolman disease, among others. See, e.g., orpha.net/consor/cgi-bin/Disease_Search_List.php; rarediseases.info.nih.gov/diseases. Further illustrative genes which may be delivered via the rAAV include, without limitation, glucose-6-phosphatase, associated with glycogen storage disease or deficiency type 1A (GSD1), phosphoenolpyruvate-carboxykinase (PEPCK), associated with PEPCK deficiency; cyclin-dependent kinase-like 5 (CDKL5), also known as serine/threonine kinase 9 (STK9) associated with seizures and severe neurodevelopmental impairment; galactose-1 phosphate uridyl transferase, associated with galactosemia; phenylalanine hydroxylase (PAH), associated with phenylketonuria (PKU); gene products associated with Primary Hyperoxaluria Type 1 including Hydroxyacid Oxidase 1 (GO/HAG1) and AGXT, branched chain alpha-ketoacid dehydrogenase, including BCKDH, BCKDH-E2, BAKDH-Ela, and BAKDH-Elb, associated with Maple syrup urine disease; fumarylacetoacetate hydrolase, associated with tyrosinemia type 1; methylmalonyl-CoA mutase, associated with methylmalonic acidemia; medium chain acyl CoA dehydrogenase, associated with medium chain acetyl CoA deficiency; ornithine transcarbamylase (OTC), associated with ornithine transcarbamylase deficiency; argininosuccinic acid synthetase (ASS1), associated with citrullinemia; lecithin-cholesterol acyltransferase (LCAT) deficiency; amethylmalonic acidemia (MMA); NPC1 associated with Niemann-Pick disease, type C1); propionic academia (PA); TTR associated with Transthyretin (TTR)-related Hereditary Amyloidosis; low density lipoprotein receptor (LDLR) protein, associated with familial hypercholesterolemia (FH), LDLR variant, such as those described in WO 2015/164778; PCSK9; ApoE and ApoC proteins, associated with dementia; UDP-glucouronosyltransferase, associated with Crigler-Najjar disease; adenosine deaminase, associated with severe combined immunodeficiency disease; hypoxanthine guanine phosphoribosyl transferase, associated with Gout and Lesch-Nyan syndrome; biotimidase, associated with biotimidase deficiency; alpha-galactosidase A (α-Gal A) associated with Fabry disease); beta-galactosidase (GLB1) associated with GM1 gangliosidosis; ATP7B associated with Wilson's Disease; beta-glucocerebrosidase, associated with Gaucher disease type 2 and 3; peroxisome membrane protein 70 kDa, associated with Zellweger syndrome; arylsulfatase A (ARSA) associated with metachromatic leukodystrophy, galactocerebrosidase (GALC) enzyme associated with Krabbe disease, alpha-glucosidase (GAA) associated with Pompe disease; sphingomyelinase (SMPD1) gene associated with Nieman Pick disease type A; argininosuccsinate synthase associated with adult onset type II citrullinemia (CTLN2); carbamoyl-phosphate synthase 1 (CPS1) associated with urea cycle disorders; survival motor neuron (SMN) protein, associated with spinal muscular atrophy; ceramidase associated with Farber lipogranulomatosis; b-hexosaminidase associated with GM2 gangliosidosis and Tay-Sachs and Sandhoff diseases; aspartylglucosaminidase associated with aspartyl-glucosaminuria; α-fucosidase associated with fucosidosis; α-mannosidase associated with alpha-mannosidosis; porphobilinogen deaminase, associated with acute intermittent porphyria (AIP); alpha-1 antitrypsin for treatment of alpha-1 antitrypsin deficiency (emphysema); erythropoietin for treatment of anemia due to thalassemia or to renal failure; vascular endothelial growth factor, angiopoietin-1, and fibroblast growth factor for the treatment of ischemic diseases; thrombomodulin and tissue factor pathway inhibitor for the treatment of occluded blood vessels as seen in, for example, atherosclerosis, thrombosis, or embolisms; aromatic amino acid decarboxylase (AADC), and tyrosine hydroxylase (TH) for the treatment of Parkinson's disease.

In certain embodiments, rAAVhu95 and/or rAAVhu96 comprises a transgene which encodes proteins including hormones and growth and differentiation factors including, without limitation, insulin, glucagon, glucagon-like peptide 1 (GLP-1), glucagon-like peptide 2 (GLP-2), growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), transforming growth factor α (TGFα), platelet-derived growth factor (PDGF), insulin growth factors I and II (IGF-I and IGF-II), any one of the transforming growth factor β superfamily, including TGF β, activins, inhibins, or any of the bone morphogenic proteins (BMP) BMPs 1-15, any one of the heregluin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), lysosomal acid lipase (LIPA or LAL), neurturin, agrin, any one of the family of semaphorins/collapsins, netrin-1 and netrin-2, hepatocyte growth factor (HGF), ephrins, noggin, sonic hedgehog and tyrosine hydroxylase. Other useful transgene encode lysosomal enzymes that cause mucopolysaccharidoses (MPS), including α-L-iduronidase (MPSI), iduronate sulfatase (MPSII), heparan N-sulfatase (sulfaminidase) (MPS IIIA, Sanfilippo A), α-N-acetyl-glucosaminidase (MPS IIIB, Sanfilippo B), acetyl-CoA:α-glucosaminide acetyltransferase (MPS IIIC, Sanfilippo C), N-acetylglucosamine 6-sulfatase (MPS HID, Sanfilippo D), galactose 6-sulfatase (MPS IVA, Morquio A), β-Galactosidase (MPS IVB, Morquio B), N-acetyl-galactosamine 4-sulfatase (MPS VI, Maroteaux-Lamy), f-Glucuronidase (MPS VII, Sly), and hyaluronidase (MPS IX).

In certain embodiments, the protein is encoded by a transgene sequence including a reporter sequence, which upon expression produces a detectable signal. Such reporter sequences include, without limitation, DNA sequences encoding β-lactamase, β-galactosidase (LacZ), alkaline phosphatase, thymidine kinase, green fluorescent protein (GFP), enhanced GFP (EGFP), chloramphenicol acetyltransferase (CAT), luciferase, membrane bound proteins including, for example, CD2, CD4, CD8, the influenza hemagglutinin protein, and others well known in the art, to which high affinity antibodies directed thereto exist or can be produced by conventional means, and fusion proteins comprising a membrane bound protein appropriately fused to an antigen tag domain from, among others, hemagglutinin or Myc.

In certain embodiments, rAAVhu95 and/or rAAVhu96 comprises a transgene which encodes proteins including, without limitation, glucose-6-phosphatase, associated with glycogen storage disease or deficiency type 1A (GSD1), phosphoenolpyruvate-carboxykinase (PEPCK), associated with PEPCK deficiency; cyclin-dependent kinase-like 5 (CDKL5), also known as serine/threonine kinase 9 (STK9) associated with seizures and severe neurodevelopmental impairment; (NGLYl)N-glycanase 1; galactose-1 phosphate uridyl transferase, associated with galactosemia; phenylalanine hydroxylase (PAH), associated with phenylketonuria (PKU); gene products associated with Primary Hyperoxaluria Type 1 including Hydroxyacid Oxidase 1 (GO/HAG1) and AGXT, branched chain alpha-ketoacid dehydrogenase, including BCKDH, BCKDH-E2, BAKDH-Ela, and BAKDH-Elb, associated with Maple syrup urine disease; fumarylacetoacetate hydrolase, associated with tyrosinemia type 1; methylmalonyl-CoA mutase, associated with methylmalonic acidemia; medium chain acyl CoA dehydrogenase, associated with medium chain acetyl CoA deficiency; ornithine transcarbamylase (OTC), associated with ormithine transcarbamylase deficiency; argininosuccinic acid synthetase (ASS1), associated with citrullinemia; lecithin-cholesterol acyltransferase (LCAT) deficiency; amethylmalonic acidemia (MMA); NPC1 associated with Niemann-Pick disease, type C1); propionic academia (PA); TTR associated with Transthyretin (TTR)-related Hereditary Amyloidosis; low density lipoprotein receptor (LDLR) protein, associated with familial hypercholesterolemia (FH), LDLR variant, such as those described in WO 2015/164778; PCSK9; ApoE and ApoC proteins, associated with dementia; UDP-glucouronosyltransferase, associated with Crigler-Najjar disease; adenosine deaminase, associated with severe combined immunodeficiency disease; hypoxanthine guanine phosphoribosyl transferase, associated with Gout and Lesch-Nyan syndrome; biotimidase, associated with biotimidase deficiency; alpha-galactosidase A (β-Gal A) associated with Fabry disease); beta-galactosidase (GLB1) associated with GM1 gangliosidosis; ATP7B associated with Wilson's Disease; beta-glucocerebrosidase, associated with Gaucher disease type 2 and 3; peroxisome membrane protein 70 kDa, associated with Zellweger syndrome; arylsulfatase A (ARSA) associated with metachromatic leukodystrophy, galactocerebrosidase (GALC) enzyme associated with Krabbe disease, alpha-glucosidase (GAA) associated with Pompe disease; sphingomyelinase (SMPD1) gene associated with Nieman Pick disease type A; argininosucesinate synthase associated with adult onset type II citrullinemia (CTLN2); carbamoyl-phosphate synthase 1 (CPS1) associated with urea cycle disorders; survival motor neuron (SMN) protein, associated with spinal muscular atrophy; ceramidase associated with Farber lipogranulomatosis; b-hexosaminidase associated with GM2 gangliosidosis and Tay-Sachs and Sandhoff diseases; aspartylglucosaminidase associated with aspartyl-glucosaminuria; α-fucosidase associated with fucosidosis; α-mannosidase associated with alpha-mannosidosis; porphobilinogen deaminase, associated with acute intermittent porphyria (AIP); alpha-1 antitrypsin for treatment of alpha-1 antitrypsin deficiency (emphysema); erythropoietin for treatment of anemia due to thalassemia or to renal failure; vascular endothelial growth factor, angiopoietin-1, and fibroblast growth factor for the treatment of ischemic diseases; thrombomodulin and tissue factor pathway inhibitor for the treatment of occluded blood vessels as seen in, for example, atherosclerosis, thrombosis, or embolisms; aromatic amino acid decarboxylase (AADC), and tyrosine hydroxylase (TH) for the treatment of Parkinson's disease; the beta adrenergic receptor, anti-sense to, or a mutant form of, phospholamban, the sarco(endo)plasmic reticulum adenosine triphosphatase-2 (SERCA2), and the cardiac adenylyl cyclase for the treatment of congestive heart failure; a tumor suppressor gene such as p53 for the treatment of various cancers; a cytokine such as one of the various interleukins for the treatment of inflammatory and immune disorders and cancers; dystrophin or minidystrophin and utrophin or miniutrophin for the treatment of muscular dystrophies; and, insulin or GLP-1 for the treatment of diabetes.

In certain embodiments, rAAVhu95 and/or rAAVhu96 comprises a transgene which is a cancer therapeutic. In certain embodiments, rAAVhu95 and/or rAAVhu96 comprises a transgene which is a cancer therapeutic. In certain embodiments, rAAVhu95 and/or rAAVhu96 comprises a transgene which is a cancer therapeutic useful for treatment of various forms of cancer. Including metastatic and refractory cancer. In certain embodiments, rAAVhu95 and/or rAAVhu96 comprises a transgene which is a cancer therapeutic that is useful in treatment of refractory and/or resistant cancers. In certain embodiments, rAAVhu95 and/or rAAVhu96 comprises a transgene which is a cancer therapeutic that is useful in treatment of trastuzumab-resistant cancer.

In certain embodiments, rAAVhu95 and/or rAAVhu96 comprises a transgene which is a cancer therapeutic that is useful in treatment of the primary and/or secondary Her2-positive breast cancer, primary and/or secondary Her2-positive gastric and/or primary and/or secondary Her2-positive gastric gastroesophageal junction cancer, and other HER-2 positive solid tumors and cancers. In certain embodiments, rAAVhu95 and/or rAAVhu96 comprises a transgene which encodes a protein which is an antibody. In certain embodiments, the rAAVhu95 and/or rAAVhu96 comprises a transgene which encodes a protein which is an antibody directed toward HER2. In certain embodiments, the rAAVhu95 and/or rAAVhu96 comprises a transgene which encodes a protein which is a trastuzumab antibody. See also, International Patent Application Publication No. WO 2015/164723 A1, and U.S. Provisional Patent Application No. 63/328,225, filed Apr. 6, 2022, which are incorporated herein by reference in their entireties.

As used herein the term “cancer” refers to proliferative diseases including, but not limited to, carcinoma, lymphoma, blastoma, sarcoma, leukemia, lung cancer, non-small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colorectal cancer (CRC), pancreatic cancer, breast cancer, triple-negative breast cancer, HER2-positive breast cancer, HER2-positive cancer, HER2-positive metastatic cancer, HER2-positive metastatic cancer in brain, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, CNS neoplasms, spinal axis tumors, brain stem glioma, glioblastoma multiforme, astrocytomas, schwanomas, ependymonas, medulloblastomas, meningiomas, squamous cell carcinomas, pituitary adenoma and Ewings sarcoma, melanoma, multiple myeloma, B-cell cancer (lymphoma), chronic lymphocytic leukemia (CLL), acute lymphoblastic leukemia (ALL), hairy cell leukemia, chronic myeloblastic leukemia, including refractory versions of any of the above cancers, or a combination of one or more of the above cancers.

As used herein, “refractory cancer” and/or “resistant cancer” refers to a cancer which is refractory or resistant to one or more cancer therapies, for example a cancer chemotherapy (cytotoxic chemotherapy). In certain embodiment, the refractory and/or resistant cancer is not amendable to surgical intervention. In certain embodiment, the refractory and/or resistant cancer is initially unresponsive to chemotherapy or radiation therapy. In certain embodiment, the refractory and/or resistant cancer becomes unresponsive to cancer therapeutics over time.

As used herein “trastuzumab-resistant” refers to a cancer which is refractory or resistant to trastuzumab treatment. In certain embodiments, “refractory” or “resistant” means that the cancer (i.e., HER2-positive) is non-responsive to trastuzumab following a standard course of treatment, e.g., the cancer continues to progress even after the trastuzumab treatment. In certain embodiments, the trastuzumab-resistant cancer is inherently resistant to trastuzumab treatment. In certain embodiments, the trastuzumab-resistant cancer acquires resistance, wherein cancer cells initially responded to treatment, but after some period of time no longer responded to trastuzumab treatment (i.e., refractory to treatment). In certain embodiments, the resistance is developed to a late stage therapeutic, wherein the HER2-positove tumors and non-responsive or become resistant to the trastuzumab therapy.

When assessed in vitro, in certain embodiments, the trastuzumab-resistant cancer cell is from a parental cell, which was trastuzumab sensitive, and which was treated with trastuzumab-comprising composition and/or solution either as a prior treatment or as a means of exerting selective pressure. See also, Pohlman, P. R., et al., Resistance to Trastuzumab in Breast Cancer, 2009, Clin. Cancer Res. 15(24):7479-7491; Vu, T., and Claret, F. X., Trastuzumab: updated mechanisms of action and resistance in breast cancer, 2012, Front. Oncol. 2(62); Rimawi, M. F., et al., Resistance to Anti-HER2 Therapies in Breast Cancer, 2015, American Society of Clinical Oncology Educational Book 35 (May 14, 2015) e157-e164, which are incorporated herein by reference.

As used herein, the term “CNS neoplasms” includes primary or metastatic cancers, which may be located in the brain (intracranial), meninges (connective tissue layer covering brain and spinal cord), or spinal cord. Examples of primary CNS cancers could be gliomas (which may include glioblastoma (also known as glioblastoma multiforme), astrocytomas, oligodendrogliomas, and ependymomas, and mixed gliomas), meningiomas, medulloblastomas, neuromas, and primary CNS lymphoma (in the brain, spinal cord, or meninges), among others. Examples of metastatic cancers include those originating in another tissue or organ, e.g., breast, lung, lymphoma, leukemia, melanoma (skin cancer), colon, kidney, prostate, or other types that metastasize to brain.

In certain embodiments, rAAVhu95 and/or rAAVhu96 comprises a transgene which encodes a protein which is a hypoxanthine-guanine phosphoribosyltransferase (HPRT) enzyme. See also, U.S. Provisional Application No. 63/208,280, filed Jun. 8, 2021, and U.S. Provisional Patent Application No. 63/341,699, filed May 13, 2022 which are incorporated herein by reference in their entireties.

In certain embodiments, the rAAVhu95 and/or rAAVhu96 may be used in gene editing systems, which system may involve one rAAV or co-administration of multiple rAAV stocks. For example, the rAAV may be engineered to deliver SpCas9, SaCas9, ARCUS, Cpf1 (also known as Cas12a), CjCas9, and other suitable gene editing constructs.

In certain embodiments, a rAAVhu95-based and/or rAAVhu96-based gene editing nuclease system is provided herein. The gene editing nuclease targets sites in a disease-associated gene, i.e., gene of interest.

In another embodiment, rAAVhu95 and/or rAAVhu96 is selected for use in gene suppression therapy, i.e., expression of one or more native genes is interrupted or suppressed at transcriptional or translational levels. This can be accomplished using short hairpin RNA (shRNA) or other techniques well known in the art. See, e.g., Sun et al, Int J Cancer. 2010 Feb. 1; 126(3):764-74 and O'Reilly M, et al. Am J Hum Genet. 2007 July; 81(1):127-35, which are incorporated herein by reference. In this embodiment, the transgene may be readily selected by one of skill in the art based upon the gene which is desired to be silenced. In certain embodiments, rAAVhu95 and/or rAAVhu96 comprises an expression cassette comprising at least one miRNA target sequences. In certain embodiments, rAAVhu95 and/or rAAVhu96 comprises the at least one miRNA targeting sequences, wherein the miRNA is a dorsal root ganglion (drg)-miRNA targeting sequences in the UTR, e.g., to reduce drg toxicity and/or axonopathy. See, e.g., PCT/US2019/67872, filed Dec. 20, 2019 and now published as WO 2020/132455, U.S. Provisional Patent Application No. 63/023,593, filed May 12, 2020, and U.S. Provisional Patent Application No. 63/038,488, filed Jun. 12, 2020, all entitled “Compositions for Drg-Specific Reduction of Transgene Expression”, and WO 2021/231579, published 18 Nov. 2021, which are incorporated herein in their entireties.

In another embodiment, rAAVhu95 and/or rAAVhu96 comprises a transgene wherein the transgene comprises more than one transgene. This may be accomplished using a single vector carrying two or more heterologous sequences, or using two or more AAV each carrying one or more heterologous sequences. In one embodiment, the AAV is used for gene suppression (or knockdown) and gene augmentation co-therapy. In knockdown/augmentation co-therapy, the defective copy of the gene of interest is silenced and a non-mutated copy is supplied. In one embodiment, this is accomplished using two or more co-administered vectors. See, Millington-Ward et al, Molecular Therapy, April 2011, 19(4):642-649 which is incorporated herein by reference. The transgenes may be readily selected by one of skill in the art based on the desired result.

In another embodiment, rAAVhu95 and/or rAAVhu96 comprises a transgene wherein the transgene is selected for use in gene correction therapy. This may be accomplished using, e.g., a zinc-finger nuclease (ZFN)-induced DNA double-strand break in conjunction with an exogenous DNA donor substrate. See, e.g., Ellis et al, Gene Therapy (epub January 2012) 20:35-42 which is incorporated herein by reference. The transgenes may be readily selected by one of skill in the art based on the desired result.

In one embodiment, the capsids described herein are useful in gene editing systems, such as the CRISPR-Cas dual vector system described in U.S. Provisional Patent Application Nos. 61/153,470, 62/183,825, 62/254,225 and 62/287,511, and PCT/US22/26483, filed Apr. 27, 2022, each of which is incorporated herein by reference. The capsids are also useful for delivery of other nucleases, including meganucleases.

In some embodiments, the AAVhu95 capsid, rAAVhu95 or compositions thereof is used for treating diseases, disorders, syndromes, and/or conditions. In some embodiments, the disorder is selected but not limited to cardiovascular, hepatic, endocrine or metabolic, musculoskeletal, neurological, and/or renal disorders. In certain embodiments, the disease includes various forms of cancers, including metastatic, and refractory and/or resistant cancer. In certain embodiments, the AAVhu95 capsid, rAAVhu95 or compositions thereof is for use in the manufacture of a medicament for the treatment of refractory and/or resistant cancer.

In some embodiments, the AAVhu95 capsid, rAAVhu95 or compositions thereof is for use in treating a subject diagnosed with a HER2-positive cancer. In certain embodiments, the AAVhu95 capsid, rAAVhu95 or compositions thereof is for use in treating a subject diagnosed with a HER2-positive cancer which is trastuzumab resistant. In some embodiments, the AAVhu95 capsid, rAAVhu95 or compositions thereof is for use in treating a subject having a metastatic HER2-positive breast cancer in the brain. In some embodiments, the AAVhu95 capsid, rAAVhu95 or compositions thereof is for use in treating a subject diagnosed with a HER2-positive breast cancer which is trastuzumab resistant (e.g., estrogen receptor (ER)-positive, progesterone receptor (PR)-positive, and Her2-positive). In certain embodiments, the AAVhu95 capsid, rAAVhu95 or compositions thereof is for use in treating a subject diagnosed with an ER-negative, PR-negative, and Her2-positive cancer. In certain embodiments, the AAVhu95 capsid, rAAVhu95 or compositions thereof is for use in treating a subject diagnosed with HER2/low tumor.

In certain embodiments, the indicated cardiovascular diseases, disorders, syndrome and/or conditions include, but not limited to, cardiovascular disease (associated lysophosphatidic acid, lipoprotein (a), or angiopoietin-like 3 (ANGPTL3), or apolipoprotein C—III (APOC3) encoding genes), block coagulation, thrombosis, end stage renal disease, clotting disorders (associated with Factor XI (F11) encoding gene), hypertension (angiotensinogen (AGT) encoding gene), and heart failure (angiotensinogen (AGT) encoding gene). Other conditions may include, e.g., progeria (Hutchinson-Gilford syndrome), associated with a mutation in Lamin A.

In certain embodiments, the indicated hepatic diseases, disorders, syndrome and/or conditions include, but not limited to, idiopathic pulmonary fibrosis (associated with SERPINHI /Hsp47 gene), liver disease (associated with hydroxysteroid 17-beta dehydrogenase 13 (HSDI7B13) encoding gene, non-alcoholic steatohepatitis (NASH) (associated with diacylglycerol O-acyltransferase-2 (DGAT2), hydroxysteroid 17-Beta Dehydrogenase 13 (HSD17B13), or patatin-like phospholipase domain-containing 3 (PNPLA3) encoding genes), and alcohol use disorder (associated with aldehyde dehydrogenase 2 (ALDH2) encoding gene).

In certain embodiments, the indicated musculoskeletal diseases, disorders, syndrome and/or conditions include, but not limited to, muscular dystrophy (associated with dystrophin, or integrin alpha(4) (VLA-4) (CD49D) encoding genes), Duchene muscular dystrophy (DMD) (associated with dystrophin (DMD) gene), centronuclear myopathy (associated with dynamin 2 (DNM2) encoding gene), and myotonic dystrophy (DM1) (associated with myotonic dystrophy protein kinase (DMPK) encoding gene).

In certain embodiments, the indicated endocrine or metabolic diseases, disorders, syndrome and/or conditions include, but not limited to, hypertriglyceridemia (associated with apolipoprotein C—III (APOC3), or angiopoietin-like 3 (ANGPTL3) encoding genes), lipodystrophy, hyperlipidemia (associated with apolipoprotein C—III (APOC3) encoding gene), hypercholesterolemia (associated with apolipoprotein B-100 (APOB-100), proprotein convertase subtilisin kexin type 9 (PCSK9)), or amyloidosis (associated with transthyretin (TTR) encoding gene), porphyria (associated with aminolevulinate synthase-1 (ALAS-1) encoding gene), neuropathy (associated with transthyretin (TTR) encoding gene), primary hyperoxaluria type 1 (associated with glycolate oxidase encoding gene), diabetes (associated with Glucagon receptor (GCGR) encoding gene), acromegaly (growth hormone receptor (GHR) encoding gene), alpha-1 antitrypsin deficiency (AATD) (associated with alpha-1 antitrypsin (AAT) encoding gene), propionic acidemia (propionyl-CoA carboxylase (PCCA/PCCB) encoding gene), glycogen storage disease type III (GDSIII) (associated with glycogen debranching enzyme (GSDIII) encoding gene), cardiometabolic disease (associated with asialoglycoprotein (ASGPR), Hydroxyacid Oxidase 1 (HA01), or alpha-1-antitrypsin (SERPINAI) encoding genes), methylmalonic acidemia (MMA) (associated with methylmalonyl CoA mutase (MMUT), cob(I)alamin adenosyltransferase (MAJAA orMMAB), methylmalonyl-CoA epimerase (MCEE), LMBR1 domain containing 1 (LMBRDI), or ATP-binding cassette subfamily D member 4 (ABCD4) encoding genes), glycogen storage disease type 1a (associated with Glucose-6-phosphatase catalytic subunit-related protein (G6PC) encoding gene), and phenylketonuria (PKU) (associated with phenylalanine hydroxylase (PAH) encoding gene).

In certain embodiments, the indicated neurological diseases, disorders, syndrome and/or conditions include, but not limited to, spinal muscular atrophy (SMA) (associated with survival motor neuron protein (SMN2) gene), SMN1, amyotrophic lateral sclerosis (ALS) (superoxide dismutase type 1 (SOD1), FUS RNA binding protein (FUS), microRNA-155, chromosome 9 open reading frame 72 (C9orf72), or ataxin-2 (ATXN2) genes), Huntington disease (associated with huntingtin (HTT) gene), hATTR polyneuropathy (associated with transthyretin (TTR) gene), Alzheimer's disease (associated with MAP-tau (MAPT) gene), Multiple System Atrophy (associated with alpha-synuclein (SNCA)), Parkinson's disease (associated with alpha-synuclein (SNCA), leucine rich repeat kinase 2 (LRRK2) genes), centronuclear myopathy (associated with dynamin 2 (DNM2) gene), Angelman syndrome (associated with ubiquitin protein ligase E3A (UBE3A) gene), epilepsy (associated with glycogen synthase 1 (GYSI) gene), Dravet Syndrome (associated with sodium voltage-gated channel alpha subunit 1 (SNCIA) gene), Leukodystrophy (associated with glial fibrillary acidic protein (GFAP) gene), prion disease (associated with prion protein (PRNP) gene), and Hereditary cerebral hemorrhage with amyloidosis-Dutch type (HCHWA-D) (associated with amyloid beta precursor protein (APP) gene).

In certain embodiments, the indicated renal diseases, disorders, syndrome and/or conditions include, but not limited to, Glomerulonephritis (IgA Nephropathy) (associated with complement factor B encoding gene), Alport syndrome (associated with proteins in the PPARa signaling pathway), and neuropathy (associated with apolipoprotein Li (APOL1) encoding gene) or an APOL1-associated chronic kidney disease.

Provided herein are also methods of treatment, wherein methods comprise the use of an rAAVhu95 and/or rAAVhu95 vector as described herein. In certain embodiments, provided herein is an rAAV as described herein (rAAVhu95, rAAVhu96) for use in delivering a gene product to a cardiac cell or to a cell of the central or peripheral nervous system. In certain embodiments, the rAAV is for use in transducing a CNS cell. In certain embodiments, the rAAV is for use in transducing a cardiac cell.

Additionally, provided herein is a method of delivering of a transgene to one or more target cells (e.g., cardiac, central nervous system (CNS), others as described herein) of a subject comprising administering to the subject a recombinant adeno-associated virus (AAV) vector comprising an AAVhu95 and/or AAVhu96 capsid and a vector genome comprising the transgene operably linked to regulatory sequences that direct expression of the transgene in the target cells of the CNS. In certain embodiments, the target cells of the CNS are parenchymal cells, cells of the choroid plexus, ependymal cells, astrocytes, and/or and neurons, optionally neurons of the cortex, hippocampus, and/or striatum. In certain embodiments, the transgene encodes a secreted gene product. In certain embodiments, the AAV vector is delivered intrathecally, optionally via intra-cisterna magna (ICM) injection. In certain embodiments, the AAV vector is delivered via intraparenchymal administration.

Provided herein are also uses of an rAAVhu95 and/or rAAVhu95 vector to target cells of the brain, such as astrocytes, at higher levels of transduction than achieved using an AAVhu68 vector. In certain embodiments, higher transduction levels are achieved in caudal sections of the brain, including frontal and temporal cortices. In certain embodiments, an AAVhu95 and/or AAVhu96 vector achieves higher levels of transduction, for example relative to AAVhu68, of neurons in the cortex, hippocampus, and/or striatum.

Provided herein are also uses of an rAAVhu95 and/or rAAVhu95 vector to target cells of the cardiovascular tissue, i.e., heart tissue, following systemic, i.e., intravenous delivery.

In a further embodiment, a cultured host cell containing a plasmid described herein for use in rAAV production of rAVhu95 and/or rAAVhu96 is provided.

As described herein, vectors having an AAVhu95 and/or AAVhu96 capsid are capable of transducing a variety of cell and tissue types and exhibit unique tropisms that are dependent on the route of administration. In certain embodiments, the methods include systemic administration of a AAVhu96 vector. In certain embodiments, the AAVhu96 vector is delivered via a route of administration suitable to target a particular cell or tissue type.

The compositions described herein may be used in a regimen involving co-administration of other active agents. Any suitable method or route can be used to administer such other agents. Routes of administration include, for example, systemic, oral, intravenous, intraperitoneal, subcutaneous, or intramuscular administration. Optionally, the AAV compositions described herein may also be administered by one of these routes.

As used herein, the term “host cell” may refer to the production (packaging) cell (i.e., production cell) or cell line in which a vector (e.g., a recombinant AAV or rAAV) is produced from an engineered sequence (e.g., a plasmid). In the alternative, the term “host cell” may refer to any target cell (i.e., target cell) in which expression of a gene product described herein is desired. Thus, a “host cell,” refers to a prokaryotic or eukaryotic cell (e.g., bacterial cell, human cell or insect cell) that contains exogenous or heterologous DNA that has been introduced into the cell by any means, e.g., electroporation, calcium phosphate precipitation, microinjection, transformation, viral infection, transfection, liposome delivery, membrane fusion techniques, high velocity DNA-coated pellets, viral infection and protoplast fusion. In certain embodiments herein, the term “host cell” refers to cultures of cells of various mammalian species for in vitro assessment of the compositions described herein. In other embodiments herein, the term “host cell” refers to the cells employed to generate and package the viral vector or recombinant virus.

As used herein, the terms “target cell” and “target tissue” can refer to any cell or tissue which is intended to be transduced by the subject AAV vector. The term may refer to any one or more of muscle, liver, lung, airway epithelium, central nervous system, neurons (e.g., motor neurons), eye (ocular cells), or heart. In another embodiment, the target tissue is the heart. In another embodiment, the target tissue is brain. In certain embodiments, the target cell is one or more cell type of the CNS, including but not limited to astrocytes, neurons, ependymal cells, and cells of the choroid plexus. In another embodiment, the target tissue is muscle.

As used herein, the term “mammalian subject” or “subject” includes any mammal in need of the methods of treatment described herein or prophylaxis, including particularly humans. Other mammals in need of such treatment or prophylaxis include dogs, cats, or other domesticated animals, horses, livestock, laboratory animals, including non-human primates, etc. The subject may be male or female.

With regard to the description of these inventions, it is intended that each of the compositions herein described, is useful, in another embodiment, in the methods of the invention. In addition, it is also intended that each of the compositions herein described as useful in the methods, is, in another embodiment, itself an embodiment of the invention.

It is to be noted that the term “a” or “an” refers to one or more. As such, the terms “a” (or “an”), “one or more,” and “at least one” are used interchangeably herein.

The words “comprise”, “comprises”, and “comprising” are to be interpreted inclusively rather than exclusively. The words “consist”, “consisting”, and its variants, are to be interpreted exclusively, rather than inclusively. While various embodiments in the specification are presented using “comprising” language, under other circumstances, a related embodiment is also intended to be interpreted and described using “consisting of” or “consisting essentially of” language.

“Patient” or “subject” as used herein means a mammalian animal, including a human, a veterinary or farm animal, a domestic animal or pet, and animals normally used for clinical research. In one embodiment, the subject of these methods and compositions is a human. In another embodiment, the subject is not a feline.

As used herein, the term “about” means a variability of 10% (+10%, e.g., +1, +2, 3, +4, +5, ±6, ±7, +8, ±9, +10, or values therebetween) from the reference given, unless otherwise specified.

In certain instances, the term “E+#” or the term “e+#” is used to reference an exponent.

For example, “5E10” or “5e10” is 5×10¹⁰. These terms may be used interchangeably.

As used herein, “disease”, “disorder” and “condition” are used interchangeably, to indicate an abnormal state in a subject.

Unless defined otherwise in this specification, technical and scientific terms used herein have the same meaning as commonly understood by one of ordinary skill in the art to which this invention belongs and by reference to published texts, which provide one skilled in the art with a general guide to many of the terms used in the present application.

### EXAMPLES

The following Examples are provided to illustrate various embodiments of the present invention. The Examples are not intended to limit the invention in any way.

We successfully isolated two new capsid genes, AAVhu95 and AAVhu96, with a high-fidelity amplification protocol we developed before. They belong to AAV clade F. Both are close to AAV9, but their DNA sequences are more than 20 base pairs different from AAV9, meaning the two are true natural isolates, not PCR artifacts.

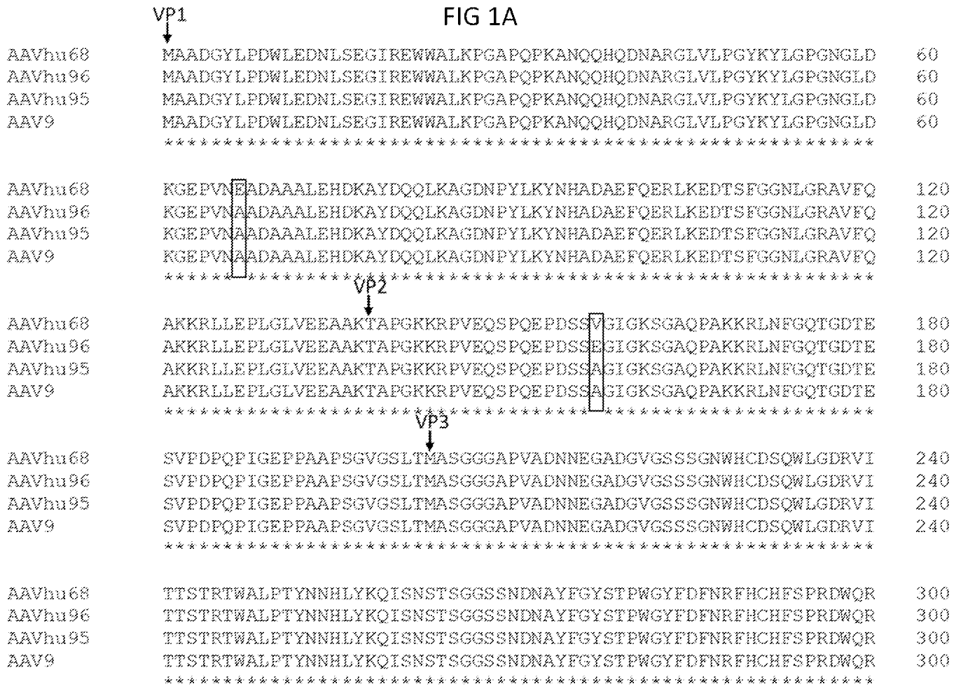

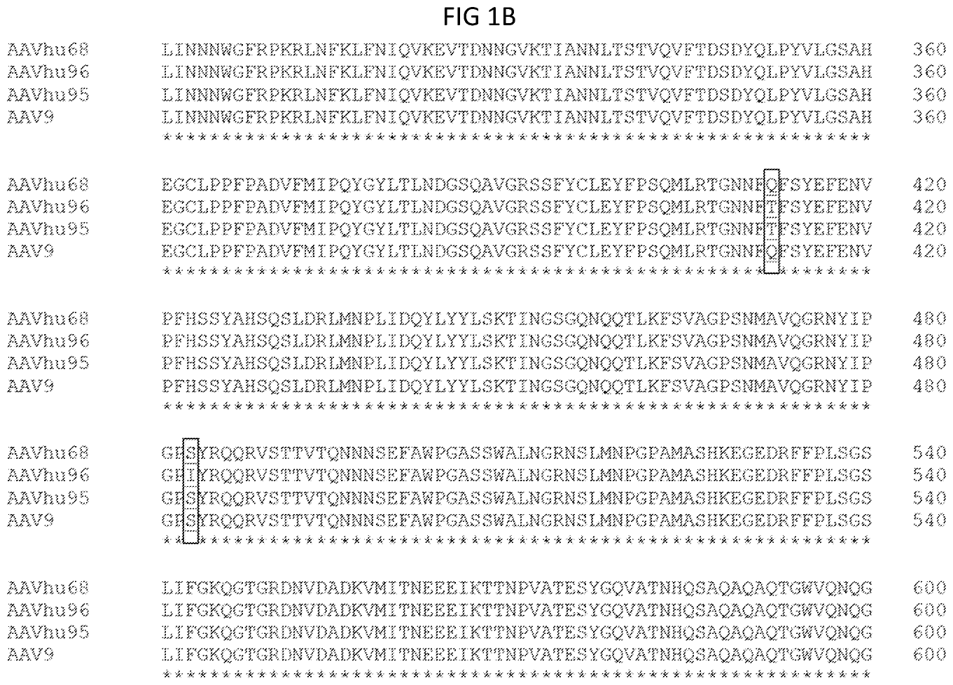

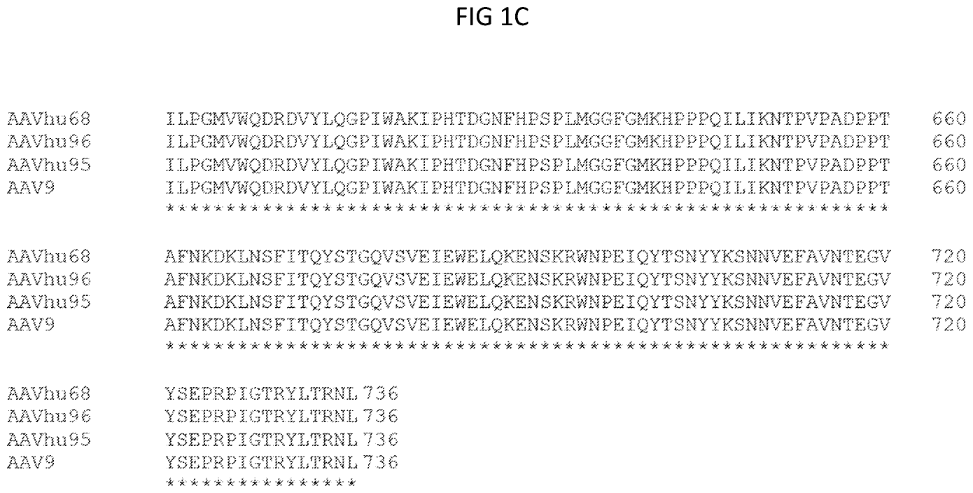

FIG. 1A shows a sequence alignment of amino acids 1 to 300 of AAV capsids of clade F: AAVhu95 (SEQ ID NO: 2), AAVhu96 (SEQ ID NO: 4), AAV9 (SEQ ID NO: 6), and AAVhu68 (SEQ ID NO: 9).

FIG. 1B shows a sequence alignment of amino acids 301 to 600 of AAV capsids of clade F: AAVhu95 (SEQ ID NO: 2), AAVhu96 (SEQ ID NO: 4), AAV9 (SEQ ID NO: 6), and AAVhu68 (SEQ ID NO: 9).

FIG. 1C shows a sequence alignment of amino acids 601 to 736 of AAV capsids of clade F: AAVhu95 (SEQ ID NO: 2), AAVhu96 (SEQ ID NO: 4), AAV9 (SEQ ID NO: 6), and AAVhu68 (SEQ ID NO: 9).

Table 2 below provides summarized overview of the amino acid differences at various positions of various capsid of Clade F (i.e., AAV9, AAVhu6S, AAVhu95, AAVhu96, AAVhu32, AAVhu31).

TABLE 2

| | AAV Clade F Capsids | | | | | |
|---|---|---|---|---|---|---|
| | AAV9 | AAVhu68 | AAVhu95 | AAVhu96 | | |
| | (SEQ ID | (SEQ ID | (SEQ ID | (SEQ ID | | |
| AAV9# | NO: 6) | NO: 9) | NO: 2) | NO: 4) | AAVhu32 | AAVhu31 |
| 14 | N | N | N | N | T | T |
| 21 | E | E | E | E | Q | Q |
| 24 | A | A | A | A | K | K |
| 29 | A | A | A | A | P | P |
| 31 | Q | Q | Q | Q | P | P |
| 34 | A | A | A | A | P | P |
| 35 | N | N | N | N | A | A |
| 36 | Q | Q | Q | Q | E | E |
| 37 | Q | Q | Q | Q | R | R |
| 39 | Q | Q | Q | Q | K | K |
| 41 | N | N | N | N | D | D |
| 42 | A | A | A | A | S | S |
| 67 | A | E | A | A | A | A |
| 157 | A | V | A | E | A | A |
| 164 | A | A | A | A | S | S |
| 170 | R | R | R | R | K | K |
| 386 | S | S | S | S | S | G |
| 412 | Q | Q | T | T | Q | Q |
| 483 | S | S | S | I | S | S |
| 716 | N | N | N | N | N | S |

### Examples 1: Materials and Methods

In this study, we engineered the nucleic acid sequence encoding AAVhu95 and AAVhu96 nucleic acid for their codons based on other AAVs' codon usage. The optimization resulted in good vector yields.

TABLE 3
DNA sequence comparison within AAV clade F.

| | | | | | |
|---|---|---|---|---|---|
| AAV9 | AAVhu68 | AAVhu95 | AAVhu96 | AAVhu31 | AAVhu32 |

| | | | | | |
|---|---|---|---|---|---|
| AAV9 | 20 | 23 | 24 | 50 | 50 |
| AAVhu68 | | 27 | 33 | 60 | 60 |
| AAVhu95 | | | 19 | 65 | 65 |
| AAVhu96 | | | | 66 | 66 |
| AAVhu31 | | | | | 6 |
| AAVhu32 | | | | | |

FIGS. 2A to 2L shows a sequence alignment of nucleic acid sequences encoding AAV capsid of Clade F, i.e., AAV9, AAVhu68, AAVhu96 and AAVhu96. FIG. 2A shows a sequence alignment of nucleotides 1 to 180 of AAV capsids of clade F: AAV9 (SEQ ID NO: 5); AAVhu68 (SEQ ID NO: 7); AAVhu68 engineered (SEQ ID NO: 8); AAVhu95 (SEQ ID NO: 10); AAVhu96 (SEQ ID NO: 11); AAVhu95 engineered (SEQ ID NO: 1); AAVhu96 (SEQ ID NO: 3). FIG. 2B shows a sequence alignment of nucleotides 181 to 360 of AAV capsids of clade F: AAV9 (SEQ ID NO: 5); AAVhu68 (SEQ ID NO: 7); AAVhu68 engineered (SEQ ID NO: 8); AAVhu95 (SEQ ID NO: 10); AAVhu96 (SEQ ID NO: 11); AAVhu95 engineered (SEQ ID NO: 1); AAVhu96 (SEQ ID NO: 3). FIG. 2C shows a sequence alignment of nucleotides 361 to 540 of AAV capsids of clade F: AAV9 (SEQ ID NO: 5); AAVhu68 (SEQ ID NO: 7); AAVhu68 engineered (SEQ ID NO: 8); AAVhu95 (SEQ ID NO: 10); AAVhu96 (SEQ ID NO: 11); AAVhu95 engineered (SEQ ID NO: 1); AAVhu96 (SEQ ID NO: 3). FIG. 2D shows a sequence alignment of nucleotides 541 to 720 of AAV capsids of clade F: AAV9 (SEQ ID NO: 5); AAVhu68 (SEQ ID NO: 7); AAVhu68 engineered (SEQ ID NO: 8); AAVhu95 (SEQ ID NO: 10); AAVhu96 (SEQ ID NO: 11); AAVhu95 engineered (SEQ ID NO: 1); AAVhu96 (SEQ ID NO: 3). FIG. 2E shows a sequence alignment of nucleotides 721 to 900 of AAV capsids of clade F: AAV9 (SEQ ID NO: 5); AAVhu68 (SEQ ID NO: 7); AAVhu68 engineered (SEQ ID NO: 8); AAVhu95 (SEQ ID NO: 10); AAVhu96 (SEQ ID NO: 11); AAVhu95 engineered (SEQ ID NO: 1); AAVhu96 (SEQ ID NO: 3). FIG. 2F shows a sequence alignment of nucleotides 901 to 1080 of AAV capsids of clade F: AAV9 (SEQ ID NO: 5); AAVhu68 (SEQ ID NO: 7); AAVhu68 engineered (SEQ ID NO: 8); AAVhu95 (SEQ ID NO: 10); AAVhu96 (SEQ ID NO: 11); AAVhu95 engineered (SEQ ID NO: 1); AAVhu96 (SEQ ID NO: 3). FIG. 2G shows a sequence alignment of nucleotides 1081 to 1260 of AAV capsids of clade F: AAV9 (SEQ ID NO: 5); AAVhu68 (SEQ ID NO: 7); AAVhu68 engineered (SEQ ID NO: 8); AAVhu95 (SEQ ID NO: 10); AAVhu96 (SEQ ID NO: 11); AAVhu95 engineered (SEQ ID NO: 1); AAVhu96 (SEQ ID NO: 3). FIG. 2H shows a sequence alignment of nucleotides 1261 to 1440 of AAV capsids of clade F: AAV9 (SEQ ID NO: 5); AAVhu68 (SEQ ID NO: 7); AAVhu68 engineered (SEQ ID NO: 8); AAVhu95 (SEQ ID NO: 10); AAVhu96 (SEQ ID NO: 11); AAVhu95 engineered (SEQ ID NO: 1); AAVhu96 (SEQ ID NO: 3). FIG. 21 shows a sequence alignment of nucleotides 1441 to 1620 of AAV capsids of cade F: AAV9 (SEQ ID NO: 5); AAVhu68 (SEQ ID NO: 7); AAVhu68 engineered (SEQ ID NO: 8); AAVhu95 (SEQ ID NO: 10); AAVhu96 (SEQ ID NO: 11); AAVhu95 engineered (SEQ ID NO: 1); AAVhu96 (SEQ ID NO: 3). FIG. 2J shows a sequence alignment of nucleotides 1621 to 1800 of AAV capsids of cade F: AAV9 (SEQ ID NO: 5); AAVhu68 (SEQ ID NO: 7); AAVhu68 engineered (SEQ ID NO: 8); AAVhu95 (SEQ ID NO: 10); AAVhu96 (SEQ ID NO: 11); AAVhu95 engineered (SEQ ID NO: 1); AAVhu96 (SEQ ID NO: 3). FIG. 2K shows a sequence alignment of nucleotides 1801 to 1980 of AAV capsids of cade F: AAV9 (SEQ ID NO: 5); AAVhu68 (SEQ ID NO: 7); AAVhu68 engineered (SEQ ID NO: 8); AAVhu95 (SEQ ID NO: 10); AAVhu96 (SEQ ID NO: 11); AAVhu95 engineered (SEQ ID NO: 1); AAVhu96 (SEQ ID NO: 3). FIG. 2L shows a sequence alignment of nucleotides 1981 to 2211 of AAV capsids of cade F: AAV9 (SEQ ID NO: 5); AAVhu68 (SEQ ID NO: 7); AAVhu68 engineered (SEQ ID NO: 8); AAVhu95 (SEQ ID NO: 10); AAVhu96 (SEQ ID NO: 11); AAVhu95 engineered (SEQ ID NO: 1); AAVhu96 (SEQ ID NO: 3).

We examined production vector yields based on One CellSTACK®cell culture vessel (Corning@) production scale for purified vectors, and compared to those of AAV9 and AAVhu68 (i.e., other Clade F). For the following study we used the Cis plasmid: pAAV.CB7.CI.eGFP.WPRE.RBG; the trans plasmid for AAVhu95: pAAV2/hu95M199 KanR; and the trans plasmid for AAVhu96: pAAV2/hu96M199 KanR (p5247).

As a further example, the rAAV are generated using triple transfection techniques, utilizing (1) a cis plasmid encoding AAV2 rep proteins and the AAVhu68/AAV9/AAVhu95/AAVhu96 VP1 cap gene (e.g., SEQ ID NO: 1, 3, 5, 7, 8), (2) a cis plasmid comprising adenovirus helper genes not provided by the packaging cell line which expresses adenovirus Ela, and (3) a trans plasmid containing the vector genome for packaging in the AAV capsid. See, e.g., US 2020/0056159.

FIG. 3 shows analysis of vector production for AAVhu95 and AAVhu96 from a one CellSTACK®cell culture vessel (Corning®) as compared with AAV9 vector yields from historical average and recent preps, plotted as GC/CS (Genome copies per CellSTACK®cell culture vessel (Corning®)). FIG. 3B shows analysis of vector production for AAVhu95 and AAVhu96 from a one CellSTACK®cell culture vessel (Corning®) as compared with AAVhu68 vector yields from historical average and recent preps, plotted as GC/CS (Genome copies per CellSTACK®cell culture vessel (Corning®)). Preliminary data analysis shows that production yields for AAVhu95 and AAVhu96 were similar to those obtained for AAV9. Furthermore, preliminary analysis shows that production yields for AAVhu95 and AAVhu96 were similar or greater than those obtained for AAVhu68.

### Examples 2: Transduction Evaluation of Novel AAV Natural Isolates in Mice

In this study, mice were injected with intravenously with rAAV comprising Clade F capsids, i.e., AAVhu95, AAVhu96 and AAVhu68, at various doses, mice were then necropsied and various tissues were examined for gene expression.

FIG. 4A shows eGFP gene expression with AAVhu95 and AAVhu96 in mouse heart tissue 14 days post-injection, as compared with AAVhu68. Mice (n=5) were administered IV with either 1×10¹¹ GC/animal or 1×10¹² GC/animal of AAVhu95.CB7.eGFP, AAVhu96.CB7.eGFP, or AAVhu68.CB7.eGFP. Mice were necropsied at day 14 post-vector administration, RNA were extracted and vector-derived sequences were quantified by quantitative reverse transcription PCR (RT-qPCR) (copy number/100 ng of total RNA). Table 4, below, provides the qualitative expression of a reporter gene in heart tissue 14 days-post injection.

TABLE 4

| Dose | | AAVhu68 | AAVhu95 | AAVhu96 |
|---|---|---|---|---|
| 1 × 10¹¹ | Median | 100% | 129% | 350% |
| | Average | 100% | 171% | 726% |
| | p | 1.000 | 0.055 | 0.174 |
| 1 × 10¹² | Median | 100% | 377% | 466% |
| | Average | 100% | 256% | 405% |
| | p | 1.000 | 0.025 | 0.018 |

FIG. 4B shows eGFP gene expression with AAVhu95 and AAVhu96 in mouse muscle tissue 14 days post-injection, as compared with AAVhu68. Mice (n=5) were administered IV with either 1×10¹¹ GC/animal or 1×10¹² GC/animal of AAVhu95.CB7.eGFP, AAVhu96.CB7.eGFP, or AAVhu68.CB7.eGFP. Mice were necropsied at day 14 post-vector administration, RNA were extracted and vector-derived sequences were quantified by quantitative reverse transcription PCR (RT-qPCR) (copy number/100 ng of total RNA). Table 5, below, provides the qualitative expression of a reporter gene in skeletal muscle tissue 14 days-post injection.

TABLE 5

| Dose | | AAVhu68 | AAVhu95 | AAVhu96 |
|---|---|---|---|---|
| 1 × 10¹¹ | Median | 100% | 285% | 273% |
| | Average | 100% | 427% | 624% |
| | p | 1.000 | 0.266 | 0.209 |
| 1 × 10¹² | Median | 100% | 47% | 69% |
| | Average | 100% | 35% | 51% |
| | p | 1.000 | 0.228 | 0.389 |

FIGS. 11A and 11B show a more detailed analysis of the qualitative expression of a reporter gene in skeletal muscle and heart tissue 14 days-post injection, as analyzed by RT-qPCR in tissue following an administration with a dose of 1×10¹² GC. FIG. 11A shows analysis of the qualitative expression of a reporter gene in skeletal muscle tissue 14 days-post injection, as analyzed by RT-qPCR in tissue following an administration with a dose of 1×10¹² GC. FIG. 11B shows analysis of the qualitative expression of a reporter gene in heart tissue 14 days-post injection, as analyzed by RT-qPCR in tissue following an administration with a dose of 1×10¹¹ GC.

These results, e.g., FIG. 4A and Table 4, show statistically higher heart expression levels for both hu95 and hu96, as compared to AAVhu68. Additionally, the results in FIG. 4B and Table 5, show higher skeletal muscle expression levels for both hu95 and hu96, as compared to AAVhu68 at a low dose of 1×10¹¹ GC/animal. Expression levels for hu95 and hu96 at the same dose levels are 3-fold or 4-fold higher than AAVhu68, respectively. This suggests that lower doses of vectors with these capsids may be required, as compared to AAVhu68.

Furthermore, eGFP gene expression was examined microscopically in necropsied tissues following administration with AAVhu68, AAVhu95 and AAVhu96. FIG. 5 shows result of the high dose barcode study of the novel clade F capsids (AAVhu95 and AAVhu96) in comparison to AAVhu68, after IV and ICM administration in NHP at a dose of 2.5×10¹³ GC/kg of each vector (total 7.5×10¹³ GC/kg). NHP were necropsied and liver, heart, skeletal muscle and brain tissue were analyzed and plotted as for relative activity (fold change normal to AAVhu68 signal). The results show statistically higher activity levels (about 2-2.5-fold) for both AAVhu95 and AAVhu96 as measured in brain tissue, when compared to AAVhu68. Additionally, the results indicate higher activity levels (about 1.5-fold) for AAVhu95 as measured in heart and skeletal muscle tissues, when compared to AAVhu68.

In another expression study, mice were administered with either AAVhu68, AAVhu95 or AAVhu96 vectors encoding enhanced green fluorescent protein (eGFP) (i.e., comprising CB7.eGFP.WPRE.rBG expression cassette) at a dose of 1×10¹ GC or 1×10¹² GC and subjected to 14-day in life examination. Mice were necropsied and tissues was analyzed using direct fluorescent microscopy, wherein representative images of various field views were taken (FIGS. 6-9), and quantified for precent of GFP positive area (FIGS. 10A-10C).

FIG. 6A shows a representative image of heart muscle tissue from GFP expression microscopy analysis following IV delivery of AAVhu68 (CB7.eGFP.WPRE.rBG) vector in mice at a dose of 1×10¹² GC. FIG. 6B shows another representative image of heart muscle tissue from GFP expression microscopy analysis following IV delivery of AAVhu68 (CB7.eGFP.WPRE.rBG) vector in mice at a dose of 1×10¹² GC. FIG. 6C shows yet another representative image of heart muscle tissue from GFP expression microscopy analysis following IV delivery of AAVhu68 (CB7.eGFP.WPRE.rBG) vector in mice at a dose of 1×10¹² GC. FIG. 6D shows yet another representative image of heart muscle tissue from GFP expression microscopy analysis following IV delivery of AAVhu68 (CB7.eGFP.WPRE.rBG) vector in mice at a dose of 1×10¹² GC. FIG. 6E shows yet another representative image of heart muscle tissue from GFP expression microscopy analysis following IV delivery of AAVhu68 (CB7.eGFP.WPRE.rBG) vector in mice at a dose of 1×10¹² GC.

FIG. 7A shows a representative image of heart muscle tissue from GFP expression microscopy analysis following IV delivery of AAVhu95 (CB7.eGFP.WPRE.rBG) vector in mice at a dose of 1×10¹2 GC. FIG. 7B shows another representative image of heart muscle tissue from GFP expression microscopy analysis following IV delivery of AAVhu95 (CB7.eGFP.WPRE.rBG) vector in mice at a dose of 1×10¹² GC. FIG. 7C shows yet another representative image of heart muscle tissue from GFP expression microscopy analysis following IV delivery of AAVhu95 (CB7.eGFP.WPRE.rBG) vector in mice at a dose of 1×10¹² GC. FIG. 7D shows yet another representative image of heart muscle tissue from GFP expression microscopy analysis following IV delivery of AAVhu95 (CB7.eGFP.WPRE.rBG) vector in mice at a dose of 1×10¹² GC. FIG. 7E shows yet another representative image of heart muscle tissue from GFP expression microscopy analysis following IV delivery of AAVhu95 (CB7.eGFP.WPRE.rBG) vector in mice at a dose of 1×10¹² GC.

FIG. 8A shows a representative image of heart muscle tissue from GFP expression microscopy analysis following IV delivery of AAVhu96 (CB7.eGFP.WPRE.rBG) vector in mice at a dose of 1×10¹² GC. FIG. 8B shows another representative image of heart muscle tissue from GFP expression microscopy analysis following IV delivery of AAVhu96 (CB7.eGFP.WPRE.rBG) vector in mice at a dose of 1×10¹² GC. FIG. 8C shows yet another representative image of heart muscle tissue from GFP expression microscopy analysis following IV delivery of AAVhu96 (CB7.eGFP.WPRE.rBG) vector in mice at a dose of 1×10¹² GC. FIG. 8D shows yet another representative image of heart muscle tissue from GFP expression microscopy analysis following IV delivery of AAVhu96 (CB7.eGFP.WPRE.rBG) vector in mice at a dose of 1×10¹² GC. FIG. 8E shows yet another representative image of heart muscle tissue from GFP expression microscopy analysis following IV delivery of AAVhu96 (CB7.eGFP.WPRE.rBG) vector in mice at a dose of 1×10¹² GC.

FIG. 10A shows analysis of quantification of GFP signal from representative microscopy images. FIG. 10A shows percent GFP-positive area in samples of analyzed tissues of heart and muscle from mice that were administered with AAVhu68.GFP, AAVhu95.GFP, or AAVhu96.GFP at a dose of 1×10¹² GC/animal. It should be noted for AAVhu95 data point, as measured in heart tissue, was indicated to be at around 0% GFP-positive area, is thought to be attributed to a possible bad injection. FIG. 10B shows percent GFP-positive area in samples of analyzed muscle tissue from mice that were administered with AAVhu68.GFP, AAVhu95.GFP, or AAVhu96.GFP at a dose of 1×10¹² GC/animal, wherein the data has been analyzed without including a data point attributed to a possible bad injection. FIG. 10C shows percent GFP-positive area in samples of analyzed heart tissue from mice that were administered with AAVhu68.GFP, AAVhu95.GFP, or AAVhu96.GFP at a dose of 1×10¹² GC/animal, wherein the data has been analyzed without including a data point attributed to a possible bad injection. These results confirm the above-mentioned RT-qPCR analysis, and show statistically higher expression levels for AAVhu95 as measured in both heart and skeletal muscle tissues, when compared to AAVhu68.

Furthermore, liver tissue samples were analyzed for the expression in mice following administration of AAVhu68.GFP, AAVhu95.GFP, or AAVhu96.GFP at a dose of 1×10¹² GC/animal. FIG. 9A shows a representative image of liver tissue from GFP expression microscopy analysis following IV delivery of AAVhu95 (CB7.eGFP.WPRE.rBG) vector in mice at a dose of 1×10¹² GC. FIG. 9B shows another representative image of liver tissue from GFP expression microscopy analysis following IV delivery of AAVhu95 (CB7.eGFP.WPRE.rBG) vector in mice at a dose of 1×10¹² GC. FIG. 9C shows yet another representative image of liver tissue from GFP expression microscopy analysis following IV delivery of AAVhu95 (CB7.eGFP.WPRE.rBG) vector in mice at a dose of 1×10¹² GC. FIG. 9D shows yet another representative image of liver tissue from GFP expression microscopy analysis following IV delivery of AAVhu95 (CB7.eGFP.WPRE.rBG) vector in mice at a dose of 1×10¹² GC. FIG. 9E shows yet another representative image of liver tissue from GFP expression microscopy analysis following IV delivery of AAVhu95 (CB7.eGFP.WPRE.rBG) vector in mice at a dose of 1×10¹² GC.

FIG. 12A shows percent GFP-positive area in samples of analyzed of tissues liver, heart and muscle from mice that were administered with AAVhu68.GFP, AAVhu95.GFP, or AAVhu96.GFP at a dose of 1×10¹² GC/animal. FIG. 12B shows percent GFP-positive area relative to liver in sample of analyzed of tissues liver, heart and muscle from mice that were administered with AAVhu68.GFP, AAVhu95.GFP, or AAVhu96.GFP at a dose of 1×10¹² GC/animal. These results show a decreased liver expression for both AAVhu95 and AAVhu96 as compared to AAVhu68 when delivered intravenously. Furthermore, the results show higher expression levels as analyzed in heart tissue in comparison to the liver tissue when administered intravenously with AAVhu96.

### Examples 3: Evaluation of Novel AAV Natural Isolates Comprising TransgeneX

In this study, we evaluated transduction efficiency of CB.CI.IL2_V1.TransgeneX.SV40 packaged into AAVhu95 capsid. Briefly, in AAVhu95 capsid evaluation study, CB.CI.IL2_V1. TransgeneX.SV40 and isotype control vector (i.e., control transgene) were packaged into AAVhu95 capsid and administered to mice via ICV injection. AAVhu95 transduction efficiency was evaluated via ProteinX expression from TransgeneX using ELISA, and via fluorescent imaging. FIG. 13 shows expression levels of expressed ProteinX encoded by TransgeneX (g/mL) as measured in serum samples at day—1, 7, 14, and 28 post administration with AAVhu95.CB.CI.IL2_V1.TransgeneX.SV40, AAVrh91.CB.CI.IL2_V1. TransgeneX.SV40 in comparison with capsid control and PBS. FIG. 14 shows expression levels of expressed ProteinX encoded by TransgeneX (g/mL) as measured in brain tissue samples at day—1, 7, 14, and 28 post administration with AAVhu95.CB.CI.IL2_V1.TransgeneX.SV40, AAVrh91.CB.CI.IL2_V1.TransgeneX.SV40 in comparison with capsid control and PBS. FIG. 15 shows vector biodistribution (GC/diploid cell) samples at day—1, 7, 14, and 28 post administration with AAVhu95.CB.CI.IL2_V1.TransgeneX.SV40, AAVrh91.CB.CI.IL2_V1. TransgeneX.SV40 in comparison with capsid control and PBS.

Additionally, we examined preclinical activity of AAVhu95.CB.CI.IL2_V1. TransgeneX.SV40 in the MDA-MB-453 disease remission and BT-474 prophylaxis model.

Briefly, in a prophylaxis model, at day—35 to—28 a guide screw was implanted, at day-21, Rag1-KO female, about 7-8 weeks-old mice were treated with AAV at a dose of 10¹¹ GC/mouse via ICV injection, at day—1 the estrogen pellet was implanted, and at day 0 the intracranial implantation of BT-474 cell line was performed. The survival was monitored until a human endpoint. FIG. 18 Kaplan-Meier survival analysis (prophylactic treatment) of probability of survival in tumor bearing mice treated with AAVhu95.CB.CI.IL2.V1.TransgeneX.SV40.

Briefly, in disease remission model on day 0 an intracranial implantation of MDA-453 luciferase+cell line was performed, and on day 3 the AAV treatment (10¹¹ GC/mouse) via ICV injection, wherein survival was examined until humane endpoint. FIG. 16 shows quantified results of the tumor bioluminescence assessment in mice xenograft (MDA-MB-453 (ER−/PR−/HER2+)) post treatment with AAVhu95.CB.CI.IL2.V1.TransgeneX.SV40 in comparison with isotype control (Isotype control: 47 days; PBS: 44 days; rAAV.TransgeneX: >6 weeks). Efficacy of rAAV.TransgeneX injected following establishment of Her2+cancer in mouse brain. Complete tumor remission was observed, as measured by tumor growth (chart) and survival (inset). FIG. 17 shows Kaplan-Meier survival analysis (disease remission) of probability of survival in tumor-bearing mice treated with AAVhu95.CB.CJ.IL2.V1. TransgeneX.SV40. These results show that a complete disease remission was achieved in tumor bearing mice treated with AAVhu95.CB. CI.IL2.V1.TransgeneX.SV40.

Furthermore, we examined preclinical activity in the BT-474 Clone 5 Trastuzumab-Resistant (ER+/PR+/HER2+) xenograft. FIG. 19A shows quantified results of the tumor bioluminescence assessment in mice xenograft (BT-474 Clone 5 Trastuzumab-Resistant (ER+/PR+/HER2+) Xenograft) post treatment with AAVhu95.CB.CI.IL2.V1.TransgeneX.SV40 in comparison with isotype control. FIG. 19B shows Kaplan-Meier survival analysis (prophylactic treatment) of probability of survival in tumor bearing mice (BT-474 Clone 5 Trastuzumab Resistant (ER+/PR+/HER2+) Xenograft) treated with AAVhu95.CB. CI.IL2.V1.TransgeneX.SV40.

Furthermore, we examined preclinical activity in the MDA-MB-231HER2/low tumor models. FIG. 20 shows Kaplan-Meier survival analysis (prophylactic treatment) of probability of survival in tumor bearing mice (MDA-MB-231HER2/low Tumors) treated with AAVhu95. CB.CI.IL2.V1.TransgeneX.SV40.

### Examples 4: Further Evaluation of AAVhu95 and AAVhu96 in Mice

In the studies described below, we utilized rAAVX comprising eGFP-expressing vector genomes packaged in the AAV capsid, as indicated AAVhu95, AAVhu96, AAVhu68, AAV9. Genome copies (GC) of rAAVgenomes (DNA) were measured by qPCR and reported as genome copies/diploid cell (GC/diploid cell). Transgene expression (RNA) was measured by RT-qPCR and reported as transcripts/100 ng total RNA. Transgene protein was measured in 3 ways: by ELISA and reported as GFP pg/ug total protein, by immunohistochemistry (tissue images with staining for transgene (brown) and individual cells (blue)), by direct fluorescence images (tissue images with green).

In one study, eGFP (GFP)-expressing vectors (AAVhu95.GFP (AAVhu96M199), AAVhu96.GFP, AAVhu68.GFP, and AAV9GFP) were administered via IV injection in mice at a dose of 1×10¹¹ GC/mouse (n=3-5/cohort). Mice were necropsied on Day 7, liver tissues were harvested, and assays were performed to examine biodistribution, eGFP expression, and eGFP histology.

FIG. 21A shows measured copies of AAV vector genomes (DNA), as measured by qPCR from liver tissue samples following intravenous administration of AAVhu68.CB7.CI.eGFP.WPRE.rBG, AAVhu95M199.CB7.CI.eGFP.WPRE.rBG, AAV9.CB7.CI.eGFP.WPRE.rBG, and plotted as genome copies/diploid cell (GC/diploid cell).

FIG. 21B shows transgene expression (RNA) as measured by RT-qPCR from liver tissue samples following intravenous administration of AAVhu68.CB7.CI.eGFP.WPRE.rBG, AAVhu95M199.CB7.CI.eGFP.WPRE.rBG, AAV9.CB7.CI.eGFP.WPRE.rBG, and plotted as transcripts/100 ng total RNA. Results of Immunofluorescent microscopy analysis confirmed the eGFP expression (results not shown).

In another study, eGFP-expressing vectors (AAVhu95.GFP (AAVhu96M199), and AAVhu68.GFP) were administered ICV injection at doses 5×10¹⁰ or 1×10¹¹ GC/mouse of AAVhu68 or AAVhu95 (n=5/cohort). Mice were necropsied on day 21, Harvest liver, brain, spleen, heart, gastrocnemius, diaphragm, spinal cord, and assays were performed to examine biodistribution, eGFP expression, and eGFP histology

FIG. 22 shows measured copies of AAV vector genomes (DNA), as measured by qPCR from tissue (liver, brain, gastroenemius, heart, diaphragm) samples following ICV administration of AAVhu68.CB7.CI.eGFP.WPRE.rBG, and AAVhu95M199.CB7.CI.eGFP.WPRE.rBG at doses of 5×10¹⁰ (5E10) and 1×10¹¹(1E11) GC/mouse, and plotted as genome copies/diploid cell (GC/diploid cell). Results of Immunohistochemistry microscopy confirmed of the eGFP expression (results not shown).

FIG. 23 shows transgene expression (RNA) as measured by RT-qPCR from liver and brain tissue samples following ICV administration of AAVhu68.CB7.CI.eGFP.WPRE.rBG, and AAVhu95M199.CB7.CI.eGFP.WPRE.rBG at doses of 5×10¹⁰ (5E10) and 1×10¹¹ (1E11) GC/mouse, and plotted as transcripts/100 ng total RNA.

### Examples 5: Further Evaluation of AAVhu95 and AAVhu96 in NHPs

In the studies described below, we utilized rAAVX comprising eGFP-expressing vector genomes packaged in the AAV capsid, as indicated AAVhu95 and AAVhu68. Genome copies (GC) of rAAVgenomes (DNA) were measured by qPCR and reported as genome copies/diploid cell (GC/diploid cell). Transgene expression (RNA) was measured by RT-qPCR and reported as transcripts/100 ng total RNA. Transgene protein was measured in 3 ways: by ELISA and reported as GFP pg/ug total protein, by immunohistochemistry (tissue images with staining for transgene (brown) and individual cells (blue)), by direct fluorescence images (tissue images with green).

In one study, eGFP (GFP)-expressing vectors (AAVhu95.GFP (AAVhu95M199.CB7.eGFP.WPRE.rBG), and AAVhu68.GFP (AAVhu68.CB7CI.eGFP.WPRE.rBG)) were administered via IV injection in Cynomologus Macaques at a dose of 5×10¹³ GC/kg (n=4 macaques (2/cohort). NHPs were necropsied on Day 14, tissue harvest of major organs was performed, and assays were performed to examine Vector DNA biodistribution, eGFP RNA expression, and eGFP histology.

FIG. 24A shows Vector DNA (GC/gDNA) biodistribution in harvested tissue samples of major organs (right, middle, and left lobes of liver, left ventricle of a heart, gastrocnemius, diaphragm, spleen) following administration of AAVhu68.CB7.CI.eGFP.WPRE.rBG, and AAVhu95M199.CB7.CI.eGFP.WPRE.rBG via IV at a dose of 5×10¹³ (5.00E+13). These results show a decrease in measured levels Vector DNA (GC/gDNA) in liver tissue following administration of AAVhu95.GFP (in comparison to AAVhu68.GFP).

FIG. 24B shows Vector DNA (GC/gDNA) biodistribution in harvested tissue samples of major organs (kidney, lung, spinal cord (cervical, thoracic, lumbar) and brain (cerebellum, cerebrum) following administration of AAVhu68.CB7.CI.eGFP.WPRE.rBG, and AAVhu95M199.CB7.CI.eGFP.WPRE.rBG via IV at a dose of 5×10¹³ (5.00E+13) in Cynomologus Macaques. These results confirm

FIG. 25A shows RNA transcript (RNA transcript/100 ng) biodistribution in harvested tissue samples of major organs (right, middle, and left lobes of liver, left ventricle of a heart, gastrocnemius, diaphragm, spleen) following administration of AAVhu68.CB7.CI.eGFP.WPRE.rBG, and AAVhu95M199.CB7.CI.eGFP.WPRE.rBG via IV at a dose of 5×10¹³ (5.00E+13) in Cynomologus Macaques.

FIG. 25B shows RNA transcript (RNA transcript/100 ng) biodistribution in harvested tissue samples of major organs (kidney, lung, spinal cord (cervical, thoracic, lumbar) and brain (cerebellum, cerebrum) following administration of AAVhu68.CB7.CI.eGFP.WPRE.rBG, and AAVhu95M199.CB7.CI.eGFP.WPRE.rBG via IV at a dose of 5×10¹³ (5.00E+13) in Cynomologus Macaques.

FIG. 26A shows eGFP expression (GFP pg/μg of protein) biodistribution in harvested tissue samples of major organs (right, middle, and left lobes of liver, left ventricle of a heart, gastrocnemius, diaphragm, spleen) following administration of AAVhu68.CB7.CI.eGFP.WPRE.rBG, and AAVhu95M199.CB7.CI.eGFP.WPRE.rBG via IV at a dose of 5×10¹³ (5.00E+13) in Cynomologus Macaques.

FIG. 26B shows eGFP expression (GFP pg/μg of protein) biodistribution in harvested tissue samples of major organs (kidney, lung, spinal cord (cervical, thoracic, lumbar) and brain (cerebellum, cerebrum) following administration of AAVhu68.CB7.CI.eGFP.WPRE.rBG, and AAVhu95M199.CB7.CI.eGFP.WPRE.rBG via IV at a dose of 5×10¹³ (5.00E+13) in Cynomologus Macaques.

FIG. 27 shows percent GFP-positive area in samples of liver, gastrocnemius, heart, and brain (cerebrum) as quantified from Immunohistochemical microscopy analysis, following administration of AAVhu68.CB7.CLeGFP.WPRE.rBG, and AAVhu95M199.CB7.CI.eGFP.WPRE.rBG via IV at a dose of 5×10¹³ (5.00E+13) in Cynomologus Macaques. These results show increased expression levels of eGFP in heart and brain (cerebrum) following AAVhu95.GFP administration as compared to AAVhu68.GFP.

Results of Immunofluorescent and/or Immunohistochemical microscopy analysis confirmed the eGFP expression levels in liver, heart, brain, spleen, gastrocnemius, diaphragm, and spinal cord (cervical, thoracis, lumbar) (results not shown).

Furthermore, similar eGFP expression levels were observed in DRG cervical tissue samples, where as lower expression levels were observed in DRG thoracic and lumbar tissue samples of macaques which were administered with AAVhu95M199.CB7.CI.eGFP.WPRE.rBG, than those administered with AAVhu68.CB7.CI.eGFP.WPRE.rBG. FIG. 29A shows a representative Immunohistochemistry (IHC) image of DRG (cervical) tissue from GFP expression analysis following IV administration of AAVhu68.CB7.CI.eGFP.WPRE.rBG vector in NHPs at a dose of 5×10¹³ GC/kg. FIG. 29B shows another representative IHC image of DRG (cervical) tissue from GFP expression analysis following IV administration of AAVhu68.CB7.CI.eGFP.WPRE.rBG vector in NHPs at a dose of 5×10¹³ GC/kg. FIG. 29C shows a representative IHC image of DRG (cervical) tissue from GFP expression analysis following IV administration of AAVhu95.CB7.CI.eGFP.WPRE.rBGvector in NHPs at a dose of 5×10¹³ GC/kg. FIG. 29D shows another representative IHC image of DRG (cervical) tissue from GFP expression analysis following IV administration of AAVhu95.CB7.CI.eGFP.WPRE.rBG vector in NHPs at a dose of 5×10¹³ GC/kg. FIG. 29E shows a representative IHC image of DRG (thoracic) tissue from GFP expression analysis following IV administration of AAVhu68.CB7.CI.eGFP.WPRE.rBG vector in NHPs at a dose of 5×10¹³ GC/kg. FIG. 29F shows another representative IHC image of DRG (thoracic) tissue from GFP expression analysis following IV administration of AAVhu68.CB7.CI.eGFP.WPRE.rBG vector in NHPs at a dose of 5×10¹³ GC/kg. FIG. 29G shows a representative IHC image of DRG (thoracic) tissue from GFP expression analysis following IV administration of AAVhu95.CB7.CI.eGFP.WPRE.rBGvector in NHPs at a dose of 5×10¹³ GC/kg. FIG. 29H shows another representative IHC image of DRG (thoracic) tissue from GFP expression analysis following IV administration of AAVhu95.CB7.CI.eGFP.WPRE.rBG vector in NHPs at a dose of 5×10¹³ GC/kg. FIG. 29I shows a representative IHC image of DRG (lumbar) tissue from GFP expression analysis following IV administration of AAVhu68.CB7.CI.eGFP.WPRE.rBG vector in NHPs at a dose of 5×10¹³ GC/kg. FIG. 29J shows another representative IHC image of DRG (lumbar) tissue from GFP expression analysis following IV administration of AAVhu68.CB7.CI.eGFP.WPRE.rBGvector in NHPs at a dose of 5×10¹³ GC/kg. FIG. 29K shows a representative IHC image of DRG (lumbar) tissue from GFP expression analysis following IV administration of AAVhu95.CB7.CI.eGFP.WPRE.rBGvector in NHPs at a dose of 5×10¹³ GC/kg. FIG. 29L shows another representative IHC image of DRG (lumbar) tissue from GFP expression analysis following IV administration of AAVhu95.CB7.CI.eGFP.WPRE.rBG vector in NHPs at a dose of 5×10¹³ GC/kg.

In another study, eGFP (GFP)-expressing vector (AAVhu95.GFP (AAVhu95M199.CB7.eGFP.WPRE.rBG) was administered via IV injection in Marmosets at a dose of 5×10¹³ GC/kg (n=3 marmosets). NHPs were necropsied on Day 14, tissue harvest of major organs was performed, and assays were performed to examine Vector DNA biodistribution, eGFP RNA expression, and eGFP histology.

FIG. 28A shows Vector DNA (GC/gDNA) biodistribution in harvested tissue samples of liver, gastrocnemius, heart and brain following administration of and AAVhu95M199.CB7.CI.eGFP.WPRE.rBG via IV at a dose of 5×10¹³ (5.00E+13) in marmosets.

FIG. 28B shows RNA transcript (RNA transcript/100 ng) biodistribution in harvested tissue samples of liver, gastrocnemius, heart and brain following administration of and AAVhu95M199.CB7.CI.eGFP.WPRE.rBG via IV at a dose of 5×10¹³ (5.00E+13) in marmosets.

Results of Immunohistochemical microscopy analysis confirmed the eGFP expression levels in liver, heart, brain, gastroenemius (results not shown).

In another study, eGFP (GFP)-expressing vector (AAVhu95.GFP (AAVhu95M199.CB7.eGFP.WPRE.rBG) is administered via ICV injection in Cynomologus Macaques at a dose 3×10¹³ GC/kg (n=3 macaques). NHPs are necropsied on Day 14, tissue harvest of major organs is performed, and assays are performed to examine Vector DNA biodistribution, eGFP RNA expression, and eGFP histology.

These results confirm the ability of AAVhu96 and AAVhu96 to transduce targeted host cells following IV administration. Furthermore, these results confirm above-mentioned (Examples 1-3) results, with vector DNA levels in liver observed to be lower in liver tissues collected post-administration of AAVhu95.GFP in comparison to AAVhu68.GFP.

All documents cited in this specification are incorporated herein by reference. The electronic sequence listing filed herewith named “UPN-22-9793PCT_20220926.xml” with size of 62,607 bytes, created on date of Sep. 26, 2022, and the contents of the electronic sequence listing (e.g., the sequences and text therein) are incorporated herein by reference in entirety. U.S. Provisional Patent Application No. 63/251,599, filed Oct. 2, 2021, and U.S. Provisional Patent Application No. 63/343,330, filed May 18, 2022, which are incorporated herein by reference in their entirety. While the invention has been described with reference to particular embodiments, it will be appreciated that modifications can be made without departing from the spirit of the invention. Such modifications are intended to fall within the scope of the appended claims.

1. A recombinant adeno-associated virus (rAAV) comprising an AAV capsid and having packaged in said AAV capsid a vector genome comprising a non-AAV exogenous nucleic acid sequence, wherein the AAV capsid is selected from:
(a) an AAVhu95 capsid which is produced from a nucleic acid sequence encoding amino acid sequence of SEQ ID NO: 2 or an amino acid sequence having at least 97% identity thereto wherein amino acid positions A67, A157, T412, and S483 of SEQ ID NO: 2 are unchanged; or
(b) an AAVhu96 capsid which is produced from a nucleic acid sequence encoding amino acid sequence of SEQ ID NO: 4 or an amino acid sequence having at least 97% identity thereto wherein amino acid positions A67, E157, T412, and 1483 of SEQ ID NO: 4 are unchanged.

2. The rAAV of claim 1, wherein the capsid is AAVhu95.

3. The rAAV of claim 1, wherein the capsid is AAVhu96.

4. The rAAV of claim 1, wherein the AAVhu95 capsid is encoded by a nucleic acid sequence of SEQ ID NO: 1 or a sequence encoding the amino acid sequence of SEQ ID NO: 2.

5. The rAAV of claim 1, wherein the AAVhu95 capsid is encoded by a nucleic acid sequence of SEQ ID NO: 1.

6. The rAAV of claim 1, wherein the AAVhu96 capsid is encoded by a nucleic acid sequence of SEQ ID NO: 3 or a sequence encoding the amino acid sequence of SEQ ID NO: 4.

7. The rAAV of claim 1, wherein the vector genome further comprising AAV 5′ inverted terminal repeat (ITR), an expression cassette, and an AAV 3′ ITR, wherein the expression cassette comprises a heterologous nucleic acid sequence operably linked to regulatory sequences which direct expression of a product encoded by the heterologous nucleic acid sequence in a target cell.

8. The rAAV of claim 7, wherein the AAV ITR sequences are from an AAV other than AAVhu95 or AAVhu96.

9. The rAAV of claim 8, wherein the AAV ITR sequences are from AAV2.

10. A recombinant adeno-associated virus (rAAV) comprising:
(A) an AAVhu95 capsid comprising one or more of:
(1) AAVhu95 capsid protein comprising:
a heterogeneous population of AAVhu95 vp1 proteins selected from: vp1 proteins produced by expression from a nucleic acid sequence which encodes the predicted amino acid sequence of 1 to 736 of SEQ ID NO: 2, vp1 proteins produced from SEQ ID NO: 1, or vp1 proteins produced from a nucleic acid sequence at least 91% identical to SEQ ID NO: 1 which encodes the predicted amino acid sequence of 1 to 736 of SEQ ID NO: 2,
a heterogeneous population of AAVhu95 vp2 proteins selected from: vp2 proteins produced by expression from a nucleic acid sequence which encodes the predicted amino acid sequence of at least about amino acids 138 to 736 of SEQ ID NO: 2 (or SEQ ID NO: 21), vp2 proteins produced from a sequence comprising at least nucleotides 412 to 2211 of SEQ ID NO: 1 (or SEQ ID NO: 13), or vp2 proteins produced from a nucleic acid sequence at least 91% identical to at least nucleotides 412 to 2211 of SEQ ID NO: 1 (or SEQ ID NO: 13) which encodes the predicted amino acid sequence of at least about amino acids 138 to 736 of SEQ ID NO: 2 (or SEQ ID NO: 21),
a heterogeneous population of AAVhu95 vp3 proteins selected from: vp3 proteins produced by expression from a nucleic acid sequence which encodes the predicted amino acid sequence of at least about amino acids 203 to 736 of SEQ ID NO: 2 (or SEQ ID NO: 22), vp3 proteins produced from a sequence comprising at least nucleotides 607 to 2211 of SEQ ID NO: 1 (or SEQ ID NO: 14), or vp3 proteins produced from a nucleic acid sequence at least 91% identical to at least nucleotides 607 to 2211 of SEQ ID NO: 1 (or SEQ ID NO: 14) which encodes the predicted amino acid sequence of at least about amino acids 203 to 736 of SEQ ID NO: 2 (or SEQ ID NO: 22); and/or
(2) a heterogeneous population of vp1 proteins which are the product of a nucleic acid sequence encoding the amino acid sequence of SEQ ID NO: 2, a heterogeneous population of vp2 proteins which are the product of a nucleic acid sequence encoding the amino acid sequence of at least about amino acids 138 to 736 of SEQ ID NO: 2 (or SEQ ID NO: 21), and a heterogeneous population of vp3 proteins which are the product of a nucleic acid sequence encoding at least amino acids 203 to 736 of SEQ ID NO: 2 (or SEQ ID NO: 22), wherein: the vp1, vp2 and vp3 proteins contain subpopulations with amino acid modifications comprising at least two highly deamidated asparagines (N) in asparagine—glycine pairs in SEQ ID NO: 2 and optionally further comprising subpopulations comprising other deamidated amino acids, wherein the deamidation results in an amino acid change; and
(B) a vector genome in the AAVhu95 capsid, the vector genome comprising a nucleic acid molecule comprising AAV inverted terminal repeat sequences and a non-AAV nucleic acid sequence encoding a product operably linked to sequences which direct expression of the product in a target cell.

11. A recombinant adeno-associated virus (rAAV) comprising:
(A) an AAVhu96 capsid comprising one or more of:
(1) AAVhu96 capsid protein comprising:
a heterogeneous population of AAVhu96 proteins selected from: vp1 proteins produced by expression from a nucleic acid sequence which encodes the predicted amino acid sequence of 1 to 736 of SEQ ID NO: 4, vp1 proteins produced from SEQ ID NO: 3, or vp1 proteins produced from a nucleic acid sequence at least 91% identical to SEQ ID NO: 3 which encodes the predicted amino acid sequence of 1 to 736 of SEQ ID NO: 4,
a heterogeneous population of AAVhu96 vp2 proteins selected from: vp2 proteins produced by expression from a nucleic acid sequence which encodes the predicted amino acid sequence of at least about amino acids 138 to 736 of SEQ ID NO: 4 (or SEQ ID NO: 23), vp2 proteins produced from a sequence comprising at least nucleotides 412 to 2211 of SEQ ID NO: 3 (or SEQ ID NO: 15), or vp2 proteins produced from a nucleic acid sequence at least 91% identical to at least nucleotides 412 to 2211 of SEQ ID NO: 3 (or SEQ ID NO: 15) which encodes the predicted amino acid sequence of at least about amino acids 138 to 736 of SEQ ID NO: 4 (or SEQ ID NO: 23),
a heterogeneous population of AAVhu96 vp3 proteins selected from: vp3 proteins produced by expression from a nucleic acid sequence which encodes the predicted amino acid sequence of at least about amino acids 203 to 736 of SEQ ID NO: 4 (or SEQ ID NO: 24), vp3 proteins produced from a sequence comprising at least nucleotides 607 to 2211 of SEQ ID NO: 3 (or SEQ ID NO: 16), or vp3 proteins produced from a nucleic acid sequence at least 91% identical to at least nucleotides 607 to 2211 of SEQ ID NO: 3 (or SEQ ID NO: 16) which encodes the predicted amino acid sequence of at least about amino acids 203 to 736 of SEQ ID NO: 4 (or SEQ ID NO: 24); and/or
(2) a heterogeneous population of vp1 proteins which are the product of a nucleic acid sequence encoding the amino acid sequence of SEQ ID NO: 4, a heterogeneous population of vp2 proteins which are the product of a nucleic acid sequence encoding the amino acid sequence of at least about amino acids 138 to 736 of SEQ ID NO: 4 (or SEQ ID NO: 23), and a heterogeneous population of vp3 proteins which are the product of a nucleic acid sequence encoding at least amino acids 203 to 736 of SEQ ID NO: 4 (or SEQ ID NO: 24), wherein the vp1, vp2 and vp3 proteins contain subpopulations with amino acid modifications comprising at least two highly deamidated asparagines (N) in asparagine—glycine pairs in SEQ ID NO: 4 and optionally further comprising subpopulations comprising other deamidated amino acids, wherein the deamidation results in an amino acid change; and
(B) a vector genome in the AAVhu96 capsid, the vector genome comprising a nucleic acid molecule comprising AAV inverted terminal repeat sequences and a non-AAV nucleic acid sequence encoding a product operably linked to sequences which direct expression of the product in a target cell.

12. A composition comprising an rAAV of claim 1 and a physiologically compatible carrier, buffer, adjuvant, and/or diluent.

13. A recombinant nucleic acid molecule comprising a promoter and an exogenous nucleic acid sequence encoding an AAVhu95 capsid protein, wherein said nucleic acid sequence is selected from SEQ ID NO: 1, nucleic acid sequence at least about 91% identical to SEQ ID NO: 1, SEQ ID NO: 10, or nucleic acid sequence at least about 99% identical to SEQ ID NO: 10.

14. A recombinant nucleic acid molecule comprising a promoter and an exogenous nucleic acid sequence encoding an AAVhu96 capsid protein, wherein said nucleic acid sequence is selected from SEQ ID NO: 3, nucleic acid sequence at least about 91% identical to SEQ ID NO: 3, SEQ ID NO: 11, or nucleic acid sequence at least about 99% identical to SEQ ID NO: 11.

15. The recombinant nucleic acid molecule of claim 13, wherein said nucleic acid molecule further comprises a nucleic acid sequence encoding a functional AAV rep protein.

16. The recombinant nucleic acid molecule of claim 15, wherein the nucleic acid sequence encoding a functional AAV rep protein is SEQ ID NO: 12.

17. The recombinant nucleic acid molecule of claim 13, wherein said recombinant nucleic acid molecule is a plasmid.

18. A production host cell comprising a recombinant nucleic acid molecule of claim 13, a nucleic acid sequence comprising an AAV vector genome, and sufficient AAV rep functions and helper functions to permit packaging of the vector genome into the AAV capsid.

19. The production host cell of claim 18, wherein the production cell is a human cell or an insect cell.

20. The production host cell of claim 18, wherein the production cell is HEK293 cell, HuH-7 cell, BHK cell, or Vero cell.

21. A method of generating a recombinant adeno-associated virus (rAAV) comprising an AAV capsid, wherein said method comprises steps of culturing production host cell comprising: (a) a molecule encoding an AAV capsid protein of AAVhu95 (amino acid sequence of SEQ ID NO: 2) or AAVhu96 (amino acid sequence SEQ ID NO: 4), (b) a functional rep gene; (c) a vector genome comprising AAV inverted terminal repeats (ITRs) and an expression cassette; and (d) sufficient helper functions to permit packaging of the vector genome into the AAV capsid protein.

22. The method of claim 21, wherein the production cell is in a suspension cell culture.

23.-24. (canceled)

25. A method of transducing a cell in a central nervous system (CNS) or peripheral nervous system, said method comprises administering an rAAV of claim 1.

26. A method of transducing a cardiac cell, said method comprises administering an rAAV of claim 1.


SEQUENCE LISTING (24 sequences)

SEQ ID NO: 1 - DNA, 2211 bp, synthetic construct
  source 1..2211: mol_type = other DNA; note = adeno-associated virus hu95 VP1 engineered nucleic acid sequence
atggctgctgacggttatcttccagattggctcgaggacaacctttctgaaggcattcgtgagtggtgggctctgaaacctggagcccctcaacccaaagcgaaccaacaacaccaggacaacgctcggggtcttgtgcttccgggttacaaatacctcggacccggtaacggactcgacaaaggagagccggtcaacgcagcggacgcggcagccctcgaacacgacaaagcctacgaccagcagctcaaggccggtgacaacccgtacctcaagtacaaccacgccgacgccgagtttcaggagcgtcttaaagaagatacgtcttttgggggcaaccttggcagagcagtcttccaggccaaaaagaggcttcttgagcctcttggtctggttgaggaagcagctaaaacggctcctggaaagaagaggcctgtagagcagtctcctcaggaaccggactcatcagcgggtattggcaaatcgggcgcacagcctgccaaaaaaagactaaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtatggatacctcaccctgaacgatggaagtcaagcggtgggacgctcatccttttactgcctggagtacttcccttcgcagatgctaaggactggaaataacttcaccttcagctatgagttcgagaacgtaccttttcacagcagctacgctcacagccagagtttggatcgcttgatgaatcctcttattgatcagtatctgtactacctgtcaaagacgattaacggttctggacagaaccaacaaacgctgaaatttagcgtggctgggcctagcaacatggctgtccagggaagaaattacatacctgggcccagctaccggcaacagagagtctcaaccactgtgactcaaaacaacaacagtgaatttgcttggcctggagccagctcttgggctctcaatggccgcaattcgctgatgaatccaggaccagctatggccagtcacaaggaaggagaagaccgttttttccctttgtctggctctctaatatttggcaaacaagggacaggaagagacaacgtggatgcggataaagtaatgattacgaacgaagaagagattaaaaccaccaatcctgtggcaacagagtcctatggacaagtggcaacaaaccaccagagcgcccaagcacaggcgcagactggctgggtccaaaaccaggggatattacctggcatggtgtggcaagatcgtgacgtgtaccttcaaggacctatctgggcaaagattcctcacacggatggaaactttcatccttctcctctgatgggaggctttggaatgaaacatccgcctcctcaaatcctcatcaaaaatactccggtaccggcagatcctccgacggccttcaacaaggacaagctgaactcatttatcactcagtactccactggacaggtcagcgtggaaattgagtgggagctacagaaagaaaacagcaaacgttggaatccagagattcagtacacttccaactactacaagtctaataatgtggaatttgctgtaaatactgaaggtgtttatagtgaacctcgccctattggaacccggtatctcacacgaaacttgtaa

SEQ ID NO: 2 - protein, 736 aa, adeno-associated virus hu95
  source 1..736: note = adeno-associated virus hu95 VP1
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFTFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 3 - DNA, 2211 bp, synthetic construct
  source 1..2211: mol_type = other DNA; note = adeno-associated virus hu96 VP1 engineered nucleic acid sequence
atggctgctgacggttatcttccagattggctcgaggacaacctttctgaaggcattcgtgagtggtgggctctgaaacctggagcccctcaacccaaagcgaaccaacaacaccaggacaacgctcggggtcttgtgcttccgggttacaaatacctcggacccggtaacggactcgacaaaggagagccggtcaacgcagcggacgcggcagccctcgaacacgacaaagcctacgaccagcagctcaaggccggtgacaacccgtacctcaagtacaaccacgccgacgccgagtttcaggagcgtcttaaagaagatacgtcttttgggggcaaccttggcagagcagtcttccaggccaaaaagaggcttcttgagcctcttggtctggttgaggaagcagctaaaacggctcctggaaagaagaggcctgtagagcagtctcctcaggaaccggactcatcagagggtattggcaaatcgggcgcacagcctgccaaaaaaagactaaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtatggatacctcaccctgaacgatggaagtcaagcggtgggacgctcatccttttactgcctggagtacttcccttcgcagatgctaaggactggaaataacttcaccttcagctatgagttcgagaacgtaccttttcacagcagctacgctcacagccagagtttggatcgcttgatgaatcctcttattgatcagtatctgtactacctgtcaaagacgattaacggttctggacagaaccaacaaacgctgaaatttagcgtggctgggcctagcaacatggctgtccagggaagaaattacatacctgggcccatctaccggcaacagagagtctcaaccactgtgactcaaaacaacaacagtgaatttgcttggcctggagccagctcttgggctctcaatggccgcaattcgctgatgaatccaggaccagctatggccagtcacaaggaaggagaagaccgttttttccctttgtctggctctctaatatttggcaaacaagggacaggaagagacaacgtggatgcggataaagtaatgattacgaacgaagaagagattaaaaccaccaatcctgtggcaacagagtcctatggacaagtggcaacaaaccaccagagcgcccaagcacaggcgcagactggctgggtccaaaaccaggggatattacctggcatggtgtggcaagatcgtgacgtgtaccttcaaggacctatctgggcaaagattcctcacacggatggaaactttcatccttctcctctgatgggaggctttggaatgaaacatccgcctcctcaaatcctcatcaaaaatactccggtaccggcagatcctccgacggccttcaacaaggacaagctgaactcatttatcactcagtactccactggacaggtcagcgtggaaattgagtgggagctacagaaagaaaacagcaaacgttggaatccagagattcagtacacttccaactactacaagtctaataatgtggaatttgctgtaaatactgaaggtgtttatagtgaacctcgccctattggaacccggtatctcacacgaaacttgtaa

SEQ ID NO: 4 - protein, 736 aa, adeno-associated virus hu96
  source 1..736: note = adeno-associated virus hu96 VP1
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSEGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFTFSYEFENVPFHSSYAHSQSLDRLMNPLIDQYLYYLSKTINGSGQNQQTLKFSVAGPSNMAVQGRNYIPGPIYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 5 - DNA, 2211 bp, adeno-associated virus 9
  source 1..2211: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 6 - protein, 736 aa, adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 7 - DNA, 2211 bp, adeno-associated virus hu68
  source 1..2211: mol_type = other DNA
atggctgccgatggttatcttccagattggctcgaggacaacctcagtgaaggcattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgaagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgtgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcaaagactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtacgggtatctaacgcttaatgatggaagccaagccgtgggtcgttcgtccttttactgcctggaatatttcccgtcgcaaatgctaagaacgggtaacaacttccagttcagctacgagtttgagaacgtacctttccatagcagctatgctcacagccaaagcctggaccgactcatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactaccaacccagtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggctttcaacaaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtttattctgaaccccgccccattggcaccagatacctgactcgtaatctgtaa

SEQ ID NO: 8 - DNA, 2211 bp, synthetic construct
  misc_feature 1..2211: note = adeno-associated virus hu68 engineered nucleic acid sequence
  source 1..2211: mol_type = other DNA
atggctgccgatggttatcttccagattggctcgaggacaacctcagtgaaggcattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgaagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgtgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcaaagactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtatggatacctcaccctgaacgacggcagtcaggcggtgggccgctcatccttctactgcctggagtacttcccttcgcagatgctgaggactggcaacaacttccagttcagctacgagttcgagaacgtccctttccacagcagctacgcccacagccagagtttggaccgcttgatgaaccctctgatcgaccagtacctgtactacctgtcaaagacgatcaacggttctggccagaaccagcagacgctgaagttcagcgtggccgggcctagcaacatggccgtccagggcagaaactacatccctgggcccagctaccggcagcagagagtctcaaccactgtgactcagaacaacaacagtgagttcgcctggcctggcgccagctcttgggccctcaacggccgcaactcgctgatgaacccaggcccagccatggccagtcacaaggagggcgaggaccgtttcttccctttgtctggctctctgatcttcggcaagcaggggaccggcagagacaacgtggacgcggacaaggtcatgatcacgaacgaggaggagatcaagaccaccaaccctgtggcaaccgagtcctacggccaggtggcaaccaaccaccagagcgcccaggcacaggcgcagactggctgggtccagaaccaggggatcctgcctggcatggtgtggcaggaccgtgacgtgtacctgcagggccctatctgggcaaagatccctcacacggacggcaacttccacccttctcctctgatgggcggcttcggcatgaagcacccgcctcctcagatcctcatcaagaacactccggtcccggcagaccctccgacggccttcaacaaggacaagctgaactcattcatcactcagtactccactggccaggtcagcgtggagatcgagtgggagctgcagaaggagaacagcaagcgttggaacccagagatccagtacacttccaactactacaagtctaacaacgtggagttcgccgtcaacactgagggtgtgtacagtgagcctcgccctatcggcacccggtacctcacccgaaacttgtga

SEQ ID NO: 9 - protein, 736 aa, adeno-associated virus hu68
MAADGYLPDWLEDNLSEGIREWWALKPGAPQPKANQQHQDNARGLVLPGYKYLGPGNGLDKGEPVNEADAAALEHDKAYDQQLKAGDNPYLKYNHADAEFQERLKEDTSFGGNLGRAVFQAKKRLLEPLGLVEEAAKTAPGKKRPVEQSPQEPDSSVGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 10 - DNA, 2211 bp, adeno-associated virus hu95
  source 1..2211: mol_type = other DNA; note = adeno-associated virus hu95 VP1
atggctgccgatggttatcttccagattggctcgaggacaaccttagtgaaggaattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagtttcaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaataatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaagccgtgggtcgttcgtccttttactgcctggaatatttcccgtcgcaaatgcttcgtaccggaaacaactttaccttcagctacgagtttgagaacgtacctttccatagcagctatgctcacagccaaagcctggaccgtctcatgaatccactcatcgaccaatacttgtactatctctcaaagactattaacggttctggacagaatcaacaaacgctaaaattcagtgtggccggacccagcaacatggcagtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcttatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggctttcaacaaggacaagctgaactctttcatcacccagtattctactggccaagttagcgtggagattgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 11 - DNA, 2211 bp, adeno-associated virus hu96
  source 1..2211: mol_type = other DNA; note = adeno-associated virus hu96 VP1
atggctgccgatggttatcttccagattggctcgaggacaaccttagtgaaggaattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgcagcagacgccgcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgagggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagttatcaccaccagcacccgaacctgggccctgcccacctacaacaaccacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgcttcgtaccggaaacaactttaccttcagctacgagtttgagaacgtacctttccatagcagctacgctcacagccaaagcctggaccgactaatgaatccactcatcgaccaatacttgtactatctctcaaagactattaacggttctgggcagaatcaacaaacgctaaaattcagtgtggccggacccagcaacatggcagtccagggaagaaactacatacctggacccatctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactaccaacccggtagcaacggagtcctatggacaagtggccacaaaccaccagagtgcccaagcacaggcgcaaacaggctgggttcaaaaccaaggaatacttccgggtatggtttggcaagacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggctttcaacaaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 12 - DNA, 1866 bp, synthetic construct
  misc_feature 1..1866: note = Rep2
  source 1..1866: mol_type = other DNA
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 13 - DNA, 1800 bp, synthetic construct
  source 1..1800: mol_type = other DNA; note = adeno-associated virus hu95 VP2 engineerd nucleic acid sequence
acggctcctggaaagaagaggcctgtagagcagtctcctcaggaaccggactcatcagcgggtattggcaaatcgggcgcacagcctgccaaaaaaagactaaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtatggatacctcaccctgaacgatggaagtcaagcggtgggacgctcatccttttactgcctggagtacttcccttcgcagatgctaaggactggaaataacttcaccttcagctatgagttcgagaacgtaccttttcacagcagctacgctcacagccagagtttggatcgcttgatgaatcctcttattgatcagtatctgtactacctgtcaaagacgattaacggttctggacagaaccaacaaacgctgaaatttagcgtggctgggcctagcaacatggctgtccagggaagaaattacatacctgggcccagctaccggcaacagagagtctcaaccactgtgactcaaaacaacaacagtgaatttgcttggcctggagccagctcttgggctctcaatggccgcaattcgctgatgaatccaggaccagctatggccagtcacaaggaaggagaagaccgttttttccctttgtctggctctctaatatttggcaaacaagggacaggaagagacaacgtggatgcggataaagtaatgattacgaacgaagaagagattaaaaccaccaatcctgtggcaacagagtcctatggacaagtggcaacaaaccaccagagcgcccaagcacaggcgcagactggctgggtccaaaaccaggggatattacctggcatggtgtggcaagatcgtgacgtgtaccttcaaggacctatctgggcaaagattcctcacacggatggaaactttcatccttctcctctgatgggaggctttggaatgaaacatccgcctcctcaaatcctcatcaaaaatactccggtaccggcagatcctccgacggccttcaacaaggacaagctgaactcatttatcactcagtactccactggacaggtcagcgtggaaattgagtgggagctacagaaagaaaacagcaaacgttggaatccagagattcagtacacttccaactactacaagtctaataatgtggaatttgctgtaaatactgaaggtgtttatagtgaacctcgccctattggaacccggtatctcacacgaaacttgtaa

SEQ ID NO: 14 - DNA, 1605 bp, synthetic construct
  source 1..1605: mol_type = other DNA; note = adeno-associated virus hu95 VP3 engineerd nucleic acid sequence
atggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtatggatacctcaccctgaacgatggaagtcaagcggtgggacgctcatccttttactgcctggagtacttcccttcgcagatgctaaggactggaaataacttcaccttcagctatgagttcgagaacgtaccttttcacagcagctacgctcacagccagagtttggatcgcttgatgaatcctcttattgatcagtatctgtactacctgtcaaagacgattaacggttctggacagaaccaacaaacgctgaaatttagcgtggctgggcctagcaacatggctgtccagggaagaaattacatacctgggcccagctaccggcaacagagagtctcaaccactgtgactcaaaacaacaacagtgaatttgcttggcctggagccagctcttgggctctcaatggccgcaattcgctgatgaatccaggaccagctatggccagtcacaaggaaggagaagaccgttttttccctttgtctggctctctaatatttggcaaacaagggacaggaagagacaacgtggatgcggataaagtaatgattacgaacgaagaagagattaaaaccaccaatcctgtggcaacagagtcctatggacaagtggcaacaaaccaccagagcgcccaagcacaggcgcagactggctgggtccaaaaccaggggatattacctggcatggtgtggcaagatcgtgacgtgtaccttcaaggacctatctgggcaaagattcctcacacggatggaaactttcatccttctcctctgatgggaggctttggaatgaaacatccgcctcctcaaatcctcatcaaaaatactccggtaccggcagatcctccgacggccttcaacaaggacaagctgaactcatttatcactcagtactccactggacaggtcagcgtggaaattgagtgggagctacagaaagaaaacagcaaacgttggaatccagagattcagtacacttccaactactacaagtctaataatgtggaatttgctgtaaatactgaaggtgtttatagtgaacctcgccctattggaacccggtatctcacacgaaacttgtaa

SEQ ID NO: 15 - DNA, 1800 bp, synthetic construct
  source 1..1800: mol_type = other DNA; note = adeno-associated virus hu96 VP2 engineerd nucleic acid sequence
acggctcctggaaagaagaggcctgtagagcagtctcctcaggaaccggactcatcagagggtattggcaaatcgggcgcacagcctgccaaaaaaagactaaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtatggatacctcaccctgaacgatggaagtcaagcggtgggacgctcatccttttactgcctggagtacttcccttcgcagatgctaaggactggaaataacttcaccttcagctatgagttcgagaacgtaccttttcacagcagctacgctcacagccagagtttggatcgcttgatgaatcctcttattgatcagtatctgtactacctgtcaaagacgattaacggttctggacagaaccaacaaacgctgaaatttagcgtggctgggcctagcaacatggctgtccagggaagaaattacatacctgggcccatctaccggcaacagagagtctcaaccactgtgactcaaaacaacaacagtgaatttgcttggcctggagccagctcttgggctctcaatggccgcaattcgctgatgaatccaggaccagctatggccagtcacaaggaaggagaagaccgttttttccctttgtctggctctctaatatttggcaaacaagggacaggaagagacaacgtggatgcggataaagtaatgattacgaacgaagaagagattaaaaccaccaatcctgtggcaacagagtcctatggacaagtggcaacaaaccaccagagcgcccaagcacaggcgcagactggctgggtccaaaaccaggggatattacctggcatggtgtggcaagatcgtgacgtgtaccttcaaggacctatctgggcaaagattcctcacacggatggaaactttcatccttctcctctgatgggaggctttggaatgaaacatccgcctcctcaaatcctcatcaaaaatactccggtaccggcagatcctccgacggccttcaacaaggacaagctgaactcatttatcactcagtactccactggacaggtcagcgtggaaattgagtgggagctacagaaagaaaacagcaaacgttggaatccagagattcagtacacttccaactactacaagtctaataatgtggaatttgctgtaaatactgaaggtgtttatagtgaacctcgccctattggaacccggtatctcacacgaaacttgtaa

SEQ ID NO: 16 - DNA, 1605 bp, synthetic construct
  source 1..1605: mol_type = other DNA; note = adeno-associated virus hu96 VP3 engineerd nucleic acid sequence
atggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtatggatacctcaccctgaacgatggaagtcaagcggtgggacgctcatccttttactgcctggagtacttcccttcgcagatgctaaggactggaaataacttcaccttcagctatgagttcgagaacgtaccttttcacagcagctacgctcacagccagagtttggatcgcttgatgaatcctcttattgatcagtatctgtactacctgtcaaagacgattaacggttctggacagaaccaacaaacgctgaaatttagcgtggctgggcctagcaacatggctgtccagggaagaaattacatacctgggcccatctaccggcaacagagagtctcaaccactgtgactcaaaacaacaacagtgaatttgcttggcctggagccagctcttgggctctcaatggccgcaattcgctgatgaatccaggaccagctatggccagtcacaaggaaggagaagaccgttttttccctttgtctggctctctaatatttggcaaacaagggacaggaagagacaacgtggatgcggataaagtaatgattacgaacgaagaagagattaaaaccaccaatcctgtggcaacagagtcctatggacaagtggcaacaaaccaccagagcgcccaagcacaggcgcagactggctgggtccaaaaccaggggatattacctggcatggtgtggcaagatcgtgacgtgtaccttcaaggacctatctgggcaaagattcctcacacggatggaaactttcatccttctcctctgatgggaggctttggaatgaaacatccgcctcctcaaatcctcatcaaaaatactccggtaccggcagatcctccgacggccttcaacaaggacaagctgaactcatttatcactcagtactccactggacaggtcagcgtggaaattgagtgggagctacagaaagaaaacagcaaacgttggaatccagagattcagtacacttccaactactacaagtctaataatgtggaatttgctgtaaatactgaaggtgtttatagtgaacctcgccctattggaacccggtatctcacacgaaacttgtaa

SEQ ID NO: 17 - DNA, 1800 bp, adeno-associated virus hu95
  source 1..1800: mol_type = other DNA; note = adeno-associated virus hu95 VP2 nucleic acid sequence
acggctcctggaaagaagaggcctgtagagcagtctcctcaggaaccggactcctccgcgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaataatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaagccgtgggtcgttcgtccttttactgcctggaatatttcccgtcgcaaatgcttcgtaccggaaacaactttaccttcagctacgagtttgagaacgtacctttccatagcagctatgctcacagccaaagcctggaccgtctcatgaatccactcatcgaccaatacttgtactatctctcaaagactattaacggttctggacagaatcaacaaacgctaaaattcagtgtggccggacccagcaacatggcagtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcttatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggctttcaacaaggacaagctgaactctttcatcacccagtattctactggccaagttagcgtggagattgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 18 - DNA, 1605 bp, adeno-associated virus hu95
  source 1..1605: mol_type = other DNA; note = adeno-associated virus hu95 VP3 nucleic acid sequence
atggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaataatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaagccgtgggtcgttcgtccttttactgcctggaatatttcccgtcgcaaatgcttcgtaccggaaacaactttaccttcagctacgagtttgagaacgtacctttccatagcagctatgctcacagccaaagcctggaccgtctcatgaatccactcatcgaccaatacttgtactatctctcaaagactattaacggttctggacagaatcaacaaacgctaaaattcagtgtggccggacccagcaacatggcagtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcttatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggctttcaacaaggacaagctgaactctttcatcacccagtattctactggccaagttagcgtggagattgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 19 - DNA, 1800 bp, adeno-associated virus hu96
  source 1..1800: mol_type = other DNA; note = adeno-associated virus hu96 VP2 nucleic acid sequence
acggctcctggaaagaagaggcctgtagagcagtctcctcaggaaccggactcctccgagggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagttatcaccaccagcacccgaacctgggccctgcccacctacaacaaccacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgcttcgtaccggaaacaactttaccttcagctacgagtttgagaacgtacctttccatagcagctacgctcacagccaaagcctggaccgactaatgaatccactcatcgaccaatacttgtactatctctcaaagactattaacggttctgggcagaatcaacaaacgctaaaattcagtgtggccggacccagcaacatggcagtccagggaagaaactacatacctggacccatctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactaccaacccggtagcaacggagtcctatggacaagtggccacaaaccaccagagtgcccaagcacaggcgcaaacaggctgggttcaaaaccaaggaatacttccgggtatggtttggcaagacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggctttcaacaaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 20 - DNA, 1605 bp, adeno-associated virus hu96
  source 1..1605: mol_type = other DNA; note = adeno-associated virus hu96 VP3 nucleic acid sequence
atggcttcaggtggtggcgcaccagtggcagacaataacgaaggtgccgatggagtgggtagttcctcgggaaattggcattgcgattcccaatggctgggggacagagttatcaccaccagcacccgaacctgggccctgcccacctacaacaaccacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgcttcgtaccggaaacaactttaccttcagctacgagtttgagaacgtacctttccatagcagctacgctcacagccaaagcctggaccgactaatgaatccactcatcgaccaatacttgtactatctctcaaagactattaacggttctgggcagaatcaacaaacgctaaaattcagtgtggccggacccagcaacatggcagtccagggaagaaactacatacctggacccatctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactaccaacccggtagcaacggagtcctatggacaagtggccacaaaccaccagagtgcccaagcacaggcgcaaacaggctgggttcaaaaccaaggaatacttccgggtatggtttggcaagacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggctttcaacaaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 21 - protein, 599 aa, adeno-associated virus hu95
  source 1..599: note = adeno-associated virus hu95 VP2
TAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFTFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 22 - protein, 534 aa, adeno-associated virus hu95
  source 1..534: note = adeno-associated virus hu95 VP3
MASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFTFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 23 - protein, 599 aa, adeno-associated virus hu96
  source 1..599: note = adeno-associated virus hu96 VP2
TAPGKKRPVEQSPQEPDSSEGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFTFSYEFENVPFHSSYAHSQSLDRLMNPLIDQYLYYLSKTINGSGQNQQTLKFSVAGPSNMAVQGRNYIPGPIYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 24 - protein, 534 aa, adeno-associated virus hu96
  source 1..534: note = adeno-associated virus hu96 VP3
MASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFTFSYEFENVPFHSSYAHSQSLDRLMNPLIDQYLYYLSKTINGSGQNQQTLKFSVAGPSNMAVQGRNYIPGPIYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL